SEQUENCE LISTING (142 sequences)

SEQ ID NO: 1 - protein, 773 aa, Thermococcus gorgonarius
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 2 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 3 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLGPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYLETTIREIEEKFGFKVLYADGDGFLATIPGADAETVKKKAKEFLDYINAKLPGLLELEYEGFYKRGFFVTKAKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 4 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLGPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYLETTIREIEEKFGFKVLYADGDGFLATIPGADAETVKKKAKEFLDYINAKLPGLLELEYEGFYKRGFFVTKAKYAVIDEEDKITTRGLEIVRRDWSEIAKETQARVLEAILKHGDVEEAVRIVKEVTEKLSKYEVPPEKLVIYRQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 5 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLGPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYLETTIREIEEKFGFKVLYADGDGFLATIPGADAETVKKKAKEFLDYINAKLPGLLELEYEGFYKRGFFVTKAKYAVIDEEDKITTRGLEIVRRNWSEIAKETQARVLEAILKHGDVEEAVRIVKEVTEKLSKYEVPPEKLVIYKQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 6 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLGPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYLETTIREIEEKFGFKVLYADGDGFLATIPGADAETVKKKAKEFLDYINAKLPGLLELEYEGFYKRGFFVTKAKYAVIDEEDKITTRGLEIVRRNWSEIAKETQARVLEAILKHGDVEEAVRIVKEVTEKLSKYEVPPEKLVIYRQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 7 - protein, 63 aa, synthetic construct
VLYXDGDGXLXXIPGAXXEXXKXXAXXXXXYINXKLXXXLELEYEGXYXRGFFXXKAKYAXXX

SEQ ID NO: 8 - protein, 63 aa, synthetic construct
VLYXDGDGXLXXIPGAXXEXXKXXAXXXXXYINXKLXXXLELEYEGXYXRGFFXXKGKYAXXX

SEQ ID NO: 9 - protein, 1107 aa, Homo sapiens
MDGKRRPGPGPGVPPKRARGGLWDDDDAPRPSQFEEDLALMEEMEAEHRLQEQEEEELQSVLEGVADGQVPPSAIDPRWLRPTPPALDPQTEPLIFQQLEIDHYVGPAQPVPGGPPPSRGSVPVLRAFGVTDEGFSVCCHIHGFAPYFYTPAPPGFGPEHMGDLQRELNLAISRDSRGGRELTGPAVLAVELCSRESMFGYHGHGPSPFLRITVALPRLVAPARRLLEQGIRVAGLGTPSFAPYEANVDFEIRFMVDTDIVGCNWLELPAGKYALRLKEKATQCQLEADVLWSDVVSHPPEGPWQRIAPLRVLSFDIECAGRKGIFPEPERDPVIQICSLGLRWGEPEPFLRLALTLRPCAPILGAKVQSYEKEEDLLQAWSTFIRIMDPDVITGYNIQNFDLPYLISRAQTLKVQTFPFLGRVAGLCSNIRDSSFQSKQTGRRDTKVVSMVGRVQMDMLQVLLREYKLRSYTLNAVSFHFLGEQKEDVQHSIITDLQNGNDQTRRRLAVYCLKDAYLPLRLLERLMVLVNAVEMARVTGVPLSYLLSRGQQVKVVSQLLRQAMHEGLLMPVVKSEGGEDYTGATVIEPLKGYYDVPIATLDFSSLYPSIMMAHNLCYTTLLRPGTAQKLGLTEDQFIRTPTGDEFVKTSVRKGLLPQILENLLSARKRAKAELAKETDPLRRQVLDGRQLALKVSANSVYGFTGAQVGKLPCLEISQSVTGFGRQMIEKTKQLVESKYTVENGYSTSAKVVYGDTDSVMCRFGVSSVAEAMALGREAADWVSGHFPSPIRLEFEKVYFPYLLISKKRYAGLLFSSRPDAHDRMDCKGLEAVRRDNCPLVANLVTASLRRLLIDRDPEGAVAHAQDVISDLLCNRIDISQLVITKELTRAASDYAGKQAHVELAERMRKRDPGSAPSLGDRVPYVIISAAKGVAAYMKSEDPLFVLEHSLPIDTQYYLEQQLAKPLLRIFEPILGEGRAEAVLLRGDHTRCKTVLTGKVGGLLAFAKRRNCCIGCRTVLSHQGAVCEFCQPRESELYQKEVSHLNALEERFSRLWTQCQRCQGSLHEDVICTSRDCPIFYMRKKVRKDLEDQEQLLRRFGPPGPEAW

SEQ ID NO: 10 - protein, 25 aa, Thermococcus gorgonarius
SLYPQYIEDTDGFFATKKKIYEQIT

SEQ ID NO: 11 - protein, 25 aa, synthetic construct
SLGPQYIEDTDGFFATKKKIYKQIT

SEQ ID NO: 12 - protein, 25 aa, synthetic construct
SLGPQYLEDTDGFLATKKKIYKQIT

SEQ ID NO: 13 - protein, 25 aa, synthetic construct
SLGPQYLEDGDGFLATKAKIYKQIT

SEQ ID NO: 14 - RNA, 30 nt, synthetic construct
  source 1..30: mol_type = other RNA
tagttggagctgrtggcgtaggcaagagtg

SEQ ID NO: 15 - RNA, 51 nt, synthetic construct
  source 1..51: mol_type = other RNA
  modified_base 1..51: mod_base = OTHER; note = 2[prime]-O-methyl-RNA
cctacgcacgtgamccatgayggacagagaaggcatgacagactgctccaa

SEQ ID NO: 16 - RNA, 27 nt, synthetic construct
  source 1..27: mol_type = other RNA
tcttacctggactmtggaatccattct

SEQ ID NO: 17 - RNA, 53 nt, synthetic construct
  source 1..53: mol_type = other RNA
  modified_base 1..53: mod_base = OTHER; note = 2[prime]-O-methyl-RNA
atggattcacgtgacccatgacggacagagaaggcatggcagacatccaggta

SEQ ID NO: 18 - RNA, 30 nt, synthetic construct
  source 1..30: mol_type = other RNA
  modified_base 1..30: mod_base = OTHER; note = 2[prime]-O-methyl-RNA
cgatatgcagtttgagaagtcgcgcattcg

SEQ ID NO: 19 - RNA, 30 nt, synthetic construct; identical to SEQ ID NO: 18
  source 1..30: mol_type = other RNA
  modified_base 3: mod_base = OTHER; note = 2[prime]-O-(2-methoxyethyl)-RNA
  modified_base 5: mod_base = OTHER; note = 2[prime]-O-(2-methoxyethyl)-RNA
  modified_base 8: mod_base = OTHER; note = 2[prime]-O-(2-methoxyethyl)-RNA
  modified_base 9: mod_base = OTHER; note = 2[prime]-O-(2-methoxyethyl)-RNA
  modified_base 15: mod_base = OTHER; note = 2[prime]-O-(2-methoxyethyl)-RNA
  modified_base 17: mod_base = OTHER; note = 2[prime]-O-(2-methoxyethyl)-RNA
  modified_base 18: mod_base = OTHER; note = 2[prime]-O-(2-methoxyethyl)-RNA
  modified_base 21: mod_base = OTHER; note = 2[prime]-O-(2-methoxyethyl)-RNA
  modified_base 23: mod_base = OTHER; note = 2[prime]-O-(2-methoxyethyl)-RNA
  modified_base 25: mod_base = OTHER; note = 2[prime]-O-(2-methoxyethyl)-RNA
  modified_base 26: mod_base = OTHER; note = 2[prime]-O-(2-methoxyethyl)-RNA
  modified_base 29: mod_base = OTHER; note = 2[prime]-O-(2-methoxyethyl)-RNA
  modified_base 2: mod_base = gm
  modified_base 4: mod_base = um
  modified_base 6: mod_base = um
  modified_base 7: mod_base = gm
  modified_base 10: mod_base = gm
  modified_base 11: mod_base = um
  modified_base 12: mod_base = um
  modified_base 13: mod_base = um
  modified_base 14: mod_base = gm
  modified_base 16: mod_base = gm
  modified_base 19: mod_base = gm
  modified_base 20: mod_base = um
  modified_base 22: mod_base = gm
  modified_base 24: mod_base = gm
  modified_base 27: mod_base = um
  modified_base 28: mod_base = um
  modified_base 30: mod_base = gm
  modified_base 1: mod_base = OTHER; note = 2[prime]-O-(2-methoxyethyl)-RNA

SEQ ID NO: 20 - RNA, 30 nt, synthetic construct; identical to SEQ ID NO: 18
  source 1..30: mol_type = other RNA
  modified_base 4: mod_base = um
  modified_base 5: mod_base = OTHER; note = 2[prime]-O-(2-methoxyethyl)-RNA
  modified_base 6: mod_base = um
  modified_base 7..10: mod_base = OTHER; note = 2[prime]-O-(2-methoxyethyl)-RNA
  modified_base 11..13: mod_base = um
  modified_base 14..19: mod_base = OTHER; note = 2[prime]-O-(2-methoxyethyl)-RNA
  modified_base 20: mod_base = um
  modified_base 21..26: mod_base = OTHER; note = 2[prime]-O-(2-methoxyethyl)-RNA
  modified_base 27..28: mod_base = um
  modified_base 29..30: mod_base = OTHER; note = 2[prime]-O-(2-methoxyethyl)-RNA
  modified_base 1..3: mod_base = OTHER; note = 2[prime]-O-(2-methoxyethyl)-RNA

SEQ ID NO: 21 - protein, 25 aa, synthetic construct
SLGPQYHEDTDGFLATKKKIYKQIT

SEQ ID NO: 22 - RNA, 12 nt, synthetic construct
  source 1..12: mol_type = other RNA
  modified_base 12: mod_base = OTHER; note = cyclic phosphate
tagttggagctg

SEQ ID NO: 23 - RNA, 53 nt, synthetic construct
  source 1..53: mol_type = other RNA
  modified_base 1..53: mod_base = OTHER; note = 2[prime]-O-methyl-RNA
atggattcncgtgacccatgacggacagagaaggcatgncanacntccaggta

SEQ ID NO: 24 - RNA, 30 nt, synthetic construct; identical to SEQ ID NO: 18
  source 1..30: mol_type = other RNA
  modified_base 27: mod_base = OTHER; note = 5-methyluracil
  modified_base 1..30: mod_base = OTHER; note = 2[prime]-O-methyl-RNA

SEQ ID NO: 25 - RNA, 30 nt, synthetic construct; identical to SEQ ID NO: 18
  source 1..30: mol_type = other RNA
  modified_base 1..30: mod_base = OTHER; note = 2[prime]-O-(2-methoxyethyl)-RNA
  modified_base 27: mod_base = OTHER; note = 5-methyluracil

SEQ ID NO: 26 - protein, 63 aa, synthetic construct
VLYXDTDGXXXXIPGAXXEXXKXXAXXXXXYINXKLXXXLELEYEGXYXRGFFXXKKKYAXXX

SEQ ID NO: 27 - protein, 63 aa, Desulfurococcus sp.
VLYADTDGFFATIPGADAETVKNKAKEFLNYINPRLPGLLELEYEGFYRRGFFVTKKKYAVID

SEQ ID NO: 28 - protein, 81 aa, Escherichia coli
VIYGDTDSTFVWLKGAHSEEEAAKIGRALVQHVNAWWAETLQKQRLTSALELEYETHFCRFLMPTIRGADTGSKKRYAGLI

SEQ ID NO: 29 - protein, 97 aa, Escherichia phage RB69
FVLYGDTDSIYVSADKIIDKVGESKFRDTNHWVDFLDKFARERMEPAIDRGFREMCEYMNNKQHLMFMDREAIAGPPLGSKGIGGFWTGKKRYALNW

SEQ ID NO: 30 - protein, 62 aa, Homo sapiens
VIYGDTDSIMINTNSTNLEEVFKLGNKVKSEVNKLYKLLEIDIDGVFKSLLLLKKKKYAALW

SEQ ID NO: 31 - protein, 54 aa, Pyrobaculum calidifontis
VIYGDTDSLFVKKSGAVDRLVKYVEERNGIEIKVDKDYERVLFTEAKKRYAGLL

SEQ ID NO: 32 - protein, 63 aa, Pyrococcus abyssi
VLYIDTDGLYATIPGAKHEEIKEKALKFVEYINSKLPGLLELEYEGFYARGFFVTKKKYALID

SEQ ID NO: 33 - protein, 63 aa, Pyrococcus furiosus
VLYIDTDGLYATIPGGESEEIKKKALEFVKYINSKLPGLLELEYEGFYKRGFFVTKKRYAVID

SEQ ID NO: 34 - protein, 63 aa, Pyrococcus sp.
VLYIDTDGLYATIPGAKPEEIKKKALEFVDYINAKLPGLLELEYEGFYVRGFFVTKKKYALID

SEQ ID NO: 35 - protein, 56 aa, Saccharolobus solfataricus
VLYGDTDSLFLLNPPKNSLENIIKWVKTTFNLDLEVDKTYKFVAFSGLKKNYFGVY

SEQ ID NO: 36 - protein, 102 aa, Saccharomyces cerevisiae
LELDTDGIWCILPKSFPETYFFTLENGKKLYLSYPCSMLNYRVHQKFTNMQYQELKDPLNYIYETHSENTIFFEVDGPYKAMILPSSKEEGKGIKKRYAVFN

SEQ ID NO: 37 - protein, 63 aa, Thermococcus gorgonarius
VLYADTDGFFATIPGADAETVKKKAKEFLDYINAKLPGLLELEYEGFYKRGFFATKKKYAVID

SEQ ID NO: 38 - protein, 63 aa, Thermococcus kodakarensis
VIYSDTDGFFATIPGADAETVKKKANEFLKYINAKLPGALELEYEGFYKRGFFVTKKKYAVID

SEQ ID NO: 39 - protein, 63 aa, Thermococcus sp.
  source 1..63: note = strain 9oN-7
VLYADTDGLHATIPGADAETVKKKAKEFLKYINPKLPGLLELEYEGFYVRGFFVTKKKYAVID

SEQ ID NO: 40 - protein, 64 aa, Vaccinia virus Copenhagen
SVYGDTDSVFTEIDSQDVDKSIEIAKELERLINNRVLFNNFKIEFEAVYKNLINQSKKKYTTNK

SEQ ID NO: 41 - protein, 25 aa, synthetic construct
SLGPQYLEDGDGFLATKAKIYRQIT

SEQ ID NO: 42 - RNA, 21 nt, synthetic construct
  source 1..21: mol_type = other RNA
tacctggactatggaatccat

SEQ ID NO: 43 - RNA, 53 nt, synthetic construct
  source 1..53: mol_type = other RNA
  modified_base 1..53: mod_base = OTHER; note = 2[prime]-O-methyl-RNA
atggattcacgtgacacatgacggacagagaaagcatggcagaaatccaggta

SEQ ID NO: 44 - RNA, 50 nt, synthetic construct
  source 1..50: mol_type = other RNA
nnnnnnagaagynnaccagagaaacacacgttgtggtacattacctggta

SEQ ID NO: 45 - DNA, 77 bp, synthetic construct
  source 1..77: mol_type = other DNA
ctcacgatgctggaccagataagcacttagccacgtagtgctgttcggtaatcgatctggcaaacgctaataagggg

SEQ ID NO: 46 - DNA, 92 bp, synthetic construct
  source 1..92: mol_type = other DNA
ccctctttcttcctcttcccgatgctggaccagataagcacttagccacgtagtgctgttcggtaatcgatctggcaaacgctaataagggg

SEQ ID NO: 47 - DNA, 20 bp, synthetic construct
  source 1..20: mol_type = other DNA
ccccttattagcgtttgcca

SEQ ID NO: 48 - RNA, 37 nt, synthetic construct
  source 1..37: mol_type = other RNA
  modified_base 1..37: mod_base = OTHER; note = 2[prime]-O-methyl-RNA
ccccttattagcgtttgccagggtaacaagacggaaa

SEQ ID NO: 49 - DNA, 18 bp, synthetic construct
  source 1..18: mol_type = other DNA
agggtaacaagacggaaa

SEQ ID NO: 50 - DNA, 96 bp, synthetic construct
  source 1..96: mol_type = other DNA
ccctagttcttcctcttcccgcctgtgccactcactatannnnnnnnnnnnnnnnnnnnnnnnnnnnnnnnnnnnnnnntttccgtcttgttaccc

SEQ ID NO: 51 - RNA, 41 nt, synthetic construct
  source 1..41: mol_type = other RNA
  modified_base 1..41: mod_base = OTHER; note = 2[prime]-O-methyl-RNA
aggagacagctatgacaaccaaggtagtgctgttcgtgggg

SEQ ID NO: 52 - DNA, 774 bp, synthetic construct
  source 1..774: mol_type = other DNA
cccctcattagcgtttgccaatgcgtaaaggaagcaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataacgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaaggcagctaaccccacgaacagcactaccttg

SEQ ID NO: 53 - DNA, 15 bp, synthetic construct
  source 1..15: mol_type = other DNA
gataagcacttagcc

SEQ ID NO: 54 - DNA, 39 bp, synthetic construct
  source 1..39: mol_type = other DNA
gactcaggtctctacgcgnnsacagatggatttttggca

SEQ ID NO: 55 - DNA, 30 bp, synthetic construct
  source 1..30: mol_type = other DNA
gactcaggtctccgcgtagaggactttaaa

SEQ ID NO: 56 - DNA, 39 bp, synthetic construct
  source 1..39: mol_type = other DNA
gactcaggtctcgcggacnnsgatggatttttggcaaca

SEQ ID NO: 57 - DNA, 30 bp, synthetic construct
  source 1..30: mol_type = other DNA
gactcaggtctcgtccgcgtagaggacttt

SEQ ID NO: 58 - DNA, 39 bp, synthetic construct
  source 1..39: mol_type = other DNA
gactcaggtctcacgaagnnsaagtacgcggttatagac

SEQ ID NO: 59 - DNA, 30 bp, synthetic construct
  source 1..30: mol_type = other DNA
gactcaggtctccttcgtcacgaagaagcc

SEQ ID NO: 60 - DNA, 39 bp, synthetic construct
  source 1..39: mol_type = other DNA
gactcaggtctcaggcgtnnstggagcgagatagcgaag

SEQ ID NO: 61 - DNA, 30 bp, synthetic construct
  source 1..30: mol_type = other DNA
gactcaggtctcacgcctaactatttcaag

SEQ ID NO: 62 - DNA, 36 bp, synthetic construct
  source 1..36: mol_type = other DNA
gactcaggtctcatctacnnscagataacccgcgac

SEQ ID NO: 63 - DNA, 30 bp, synthetic construct
  source 1..30: mol_type = other DNA
gactcaggtctcgtagatgaccagcttctc

SEQ ID NO: 64 - DNA, 72 bp, synthetic construct
  source 1..72: mol_type = other DNA
gactcaggtctcaagacaggcagcggcagcggcagccatcaccatcaccatcactaataagtcgacctgcag

SEQ ID NO: 65 - DNA, 31 bp, synthetic construct
  source 1..31: mol_type = other DNA
gactcaggtctctgtcttaggttttagccac

SEQ ID NO: 66 - DNA, 42 bp, synthetic construct
  source 1..42: mol_type = other DNA
gactcaggtctccagtaccacgagactacgataagggaaata

SEQ ID NO: 67 - DNA, 27 bp, synthetic construct
  source 1..27: mol_type = other DNA
gactcaggtctcgtactgcctgcccca

SEQ ID NO: 68 - DNA, 42 bp, synthetic construct
  source 1..42: mol_type = other DNA
gactcaggtctcgcggacggggatggatttttggcaacaata

SEQ ID NO: 69 - DNA, 42 bp, synthetic construct
  source 1..42: mol_type = other DNA
gactcaggtctcacgaaggccaagtacgcggttatagacgag

SEQ ID NO: 70 - DNA, 36 bp, synthetic construct
  source 1..36: mol_type = other DNA
gactcaggtctcatctaccggcagataacccgcgac

SEQ ID NO: 71 - RNA, 21 nt, synthetic construct
  source 1..21: mol_type = other RNA
aatctaccacatcgctcattg

SEQ ID NO: 72 - DNA, 51 bp, synthetic construct
  source 1..51: mol_type = other DNA
cgaatgcgcgacttctcaaactgcatatcgcaatgagcgatgtggtagatt

SEQ ID NO: 73 - DNA, 54 bp, synthetic construct
  source 1..54: mol_type = other DNA
gagctgaaaatgctagcattcgatattgaaactctctaccatgagggcgaggag

SEQ ID NO: 74 - DNA, 54 bp, synthetic construct
  source 1..54: mol_type = other DNA
ctcctcgccctcatggtagagagtttcaatatcgaatgctagcattttcagctc

SEQ ID NO: 75 - DNA, 40 bp, synthetic construct
  source 1..40: mol_type = other DNA
cttgagattgtgaggcgtaactggagcgagatagcgaaag

SEQ ID NO: 76 - DNA, 40 bp, synthetic construct
  source 1..40: mol_type = other DNA
ctttcgctatctcgctccagttacgcctcacaatctcaag

SEQ ID NO: 77 - DNA, 42 bp, synthetic construct
  source 1..42: mol_type = other DNA
gtaacggcctggggaagggagtacattacgatgaccatcaag

SEQ ID NO: 78 - DNA, 42 bp, synthetic construct
  source 1..42: mol_type = other DNA
cttgatggtcatcgtaatgtactcccttccccaggccgttac

SEQ ID NO: 79 - DNA, 38 bp, synthetic construct
  source 1..38: mol_type = other DNA
cagcgacaccgacggattttttgccacaatacctggag

SEQ ID NO: 80 - DNA, 38 bp, synthetic construct
  source 1..38: mol_type = other DNA
ctccaggtattgtggcaaaaaatccgtcggtgtcgctg

SEQ ID NO: 81 - DNA, 39 bp, synthetic construct
  source 1..39: mol_type = other DNA
gacgttctcataacctacgatggtgacaacttcgacttc

SEQ ID NO: 82 - DNA, 39 bp, synthetic construct
  source 1..39: mol_type = other DNA
gaagtcgaagttgtcaccatcgtaggttatgagaacgtc

SEQ ID NO: 83 - DNA, 47 bp, synthetic construct
  source 1..47: mol_type = other DNA
ctactttactcatccacaggacgtgccagcaataagggacaagatac

SEQ ID NO: 84 - DNA, 47 bp, synthetic construct
  source 1..47: mol_type = other DNA
gtatcttgtcccttattgctggcacgtcctgtggatgagtaaagtag

SEQ ID NO: 85 - DNA, 52 bp, synthetic construct
  source 1..52: mol_type = other DNA
tcgatactggtactaatgattaacgatgttggcaaacgctaataaggggtcg

SEQ ID NO: 86 - DNA, 40 bp, synthetic construct
  source 1..40: mol_type = other DNA
gatcgatctcgcccgcgaaattaatacgactcactatagg

SEQ ID NO: 87 - DNA, 35 bp, synthetic construct
  source 1..35: mol_type = other DNA
gcggctcttcctctctcatccgccggccgactagt

SEQ ID NO: 88 - RNA, 30 nt, synthetic construct
  source 1..30: mol_type = other RNA
tagttggagctgatggcgtaggcaagagtg

SEQ ID NO: 89 - DNA, 90 bp, synthetic construct
  source 1..90: mol_type = other DNA
tggcgtaggcaagagtgctacgccnnnnnnnnnnnnnnnnnnnnnnnnnnnnnnnnnnnnnnnnctccaactcaacgaccagtacacaag

SEQ ID NO: 90 - DNA, 35 bp, synthetic construct
  source 1..35: mol_type = other DNA
ctctgctactcgtggtccttgtgtactggtcgttg

SEQ ID NO: 91 - DNA, 17 bp, synthetic construct
  source 1..17: mol_type = other DNA
tggcgtaggcaagagtg

SEQ ID NO: 92 - DNA, 17 bp, synthetic construct
  source 1..17: mol_type = other DNA
ctctgctactcgtggtc

SEQ ID NO: 93 - DNA, 18 bp, synthetic construct
  source 1..18: mol_type = other DNA
cttgtgtactggtcgttg

SEQ ID NO: 94 - DNA, 85 bp, synthetic construct
  source 1..85: mol_type = other DNA
cttgtgtactggtcgttgggttggagcagtctgtcatgccttctctgtccatcatggttcacgtgcgtcactcttgcctacgcca

SEQ ID NO: 95 - RNA, 51 nt, synthetic construct
  source 1..51: mol_type = other RNA
  modified_base 1..51: mod_base = OTHER; note = 2[prime]-O-methyl-RNA
cctacgcacgtgacccatgacggacagagaaggcatgacagactgctccaa

SEQ ID NO: 96 - DNA, 28 bp, synthetic construct
  source 1..28: mol_type = other DNA
cgccatggctagctacaacgaagctcca

SEQ ID NO: 97 - RNA, 30 nt, synthetic construct
  source 1..30: mol_type = other RNA
tagttggagctggtggcgtaggcaagagtg

SEQ ID NO: 98 - RNA, 30 nt, synthetic construct; identical to SEQ ID NO: 88
  source 1..30: mol_type = other RNA

SEQ ID NO: 99 - RNA, 21 nt, synthetic construct
  source 1..21: mol_type = other RNA
aatctaccacatcgctcattg

SEQ ID NO: 100 - DNA, 74 bp, synthetic construct
  source 1..74: mol_type = other DNA
tacctggaagtctgtcatgccttctctgtccgtcatgggtcacgtgaatccatcaatgagcgatgtggtagatt

SEQ ID NO: 101 - DNA, 74 bp, synthetic construct
  source 1..74: mol_type = other DNA
tacctggaagtctgtcatgccttctctgtccgtcatgggtcacgggaatccatcaatgagcgatgtggtagatt

SEQ ID NO: 102 - DNA, 74 bp, synthetic construct
  source 1..74: mol_type = other DNA
tacctggaagtctgtcatgccttctctgtccgtcatgggtcacgcgaatccatcaatgagcgatgtggtagatt

SEQ ID NO: 103 - DNA, 74 bp, synthetic construct
  source 1..74: mol_type = other DNA
tacctggaagtctgtcatgccttctctgtccgtcatgggtcacgagaatccatcaatgagcgatgtggtagatt

SEQ ID NO: 104 - DNA, 74 bp, synthetic construct
  source 1..74: mol_type = other DNA
tacctggaagtctggcatgccttctctgtccgtcatgggtcacgtgaatccatcaatgagcgatgtggtagatt

SEQ ID NO: 105 - DNA, 74 bp, synthetic construct
  source 1..74: mol_type = other DNA
tacctggaagtctgccatgccttctctgtccgtcatgggtcacgtgaatccatcaatgagcgatgtggtagatt

SEQ ID NO: 106 - DNA, 74 bp, synthetic construct
  source 1..74: mol_type = other DNA
tacctggaagtctgacatgccttctctgtccgtcatgggtcacgtgaatccatcaatgagcgatgtggtagatt

SEQ ID NO: 107 - DNA, 74 bp, synthetic construct
  source 1..74: mol_type = other DNA
tacctggaagtttgtcatgccttctctgtccgtcatgggtcacgtgaatccatcaatgagcgatgtggtagatt

SEQ ID NO: 108 - DNA, 74 bp, synthetic construct
  source 1..74: mol_type = other DNA
tacctggaagtgtgtcatgccttctctgtccgtcatgggtcacgtgaatccatcaatgagcgatgtggtagatt

SEQ ID NO: 109 - DNA, 74 bp, synthetic construct
  source 1..74: mol_type = other DNA
tacctggaagtatgtcatgccttctctgtccgtcatgggtcacgtgaatccatcaatgagcgatgtggtagatt

SEQ ID NO: 110 - DNA, 74 bp, synthetic construct
  source 1..74: mol_type = other DNA
tacctggatgtctgtcatgccttctctgtccgtcatgggtcacgtgaatccatcaatgagcgatgtggtagatt

SEQ ID NO: 111 - DNA, 74 bp, synthetic construct
  source 1..74: mol_type = other DNA
tacctggaggtctgtcatgccttctctgtccgtcatgggtcacgtgaatccatcaatgagcgatgtggtagatt

SEQ ID NO: 112 - DNA, 74 bp, synthetic construct
  source 1..74: mol_type = other DNA
tacctggacgtctgtcatgccttctctgtccgtcatgggtcacgtgaatccatcaatgagcgatgtggtagatt

SEQ ID NO: 113 - DNA, 74 bp, synthetic construct
  source 1..74: mol_type = other DNA
tacctggatgtctgccatgccttctctgtccgtcatgggtcacgtgaatccatcaatgagcgatgtggtagatt

SEQ ID NO: 114 - RNA, 53 nt, synthetic construct
  source 1..53: mol_type = other RNA
  modified_base 1..53: mod_base = OTHER; note = 2[prime]-O-methyl-RNA
atggattcacgtgacccatgacggacagagaaggcatgacagacatccaggta

SEQ ID NO: 115 - RNA, 27 nt, synthetic construct
  source 1..27: mol_type = other RNA
tcttacctggactctggaatccattct

SEQ ID NO: 116 - RNA, 27 nt, synthetic construct
  source 1..27: mol_type = other RNA
tcttacctggactatggaatccattct

SEQ ID NO: 117 - DNA, 31 bp, synthetic construct
  source 1..31: mol_type = other DNA
tggtagttggagctgatggcgtaggcaagag

SEQ ID NO: 118 - DNA, 31 bp, synthetic construct
  source 1..31: mol_type = other DNA
ctcttgcctacgccatcagctccaactacca

SEQ ID NO: 119 - DNA, 31 bp, synthetic construct
  source 1..31: mol_type = other DNA
tagttggagctggtgacgtaggcaagagtgc

SEQ ID NO: 120 - DNA, 31 bp, synthetic construct
  source 1..31: mol_type = other DNA
gcactcttgcctacgtcaccagctccaacta

SEQ ID NO: 121 - DNA, 36 bp, synthetic construct
  source 1..36: mol_type = other DNA
agcaacagtcttacctggactatggaatccattctg

SEQ ID NO: 122 - DNA, 36 bp, synthetic construct
  source 1..36: mol_type = other DNA
cagaatggattccatagtccaggtaagactgttgct

SEQ ID NO: 123 - DNA, 84 bp, synthetic construct
  source 1..84: mol_type = other DNA
aatgatacggcgaccaccgagatctacactctttccctacacgacgctcttccgatctnnnattccttggcgtaggcaagagtg

SEQ ID NO: 124 - DNA, 79 bp, synthetic construct
  source 1..79: mol_type = other DNA
caagcagaagacggcatacgagatcggtctcggcattcctgctgaaccgctcttccgatctcttgtgtactggtcgttg

SEQ ID NO: 125 - DNA, 74 bp, synthetic construct
  source 1..74: mol_type = other DNA
tacctggatgtctgccatgctttctctgtccgtcatgtgtcacgtgaatccatcaatgagcgatgtggtagatt

SEQ ID NO: 126 - DNA, 74 bp, synthetic construct
  source 1..74: mol_type = other DNA
tacctggatttctgccatgctttctctgtccgtcatgtgtcacgtgaatccatcaatgagcgatgtggtagatt

SEQ ID NO: 127 - DNA, 74 bp, synthetic construct
  source 1..74: mol_type = other DNA
tacctggatttcttccatgctttctctgtccgtcatgtgtcacgtgaatccatcaatgagcgatgtggtagatt

SEQ ID NO: 128 - DNA, 66 bp, synthetic construct
  source 1..66: mol_type = other DNA
cctatcgcggtatcggaatcccnnnnnnnnnnnnnnnnnnnnnnnnncttccgaggatgaacttga

SEQ ID NO: 129 - DNA, 63 bp, synthetic construct
  source 1..63: mol_type = other DNA
acatctcgtgcgcatctgcannnnnnnnnnnnnnnnnnnnnnnnntacaacggcgacaacttc

SEQ ID NO: 130 - DNA, 87 bp, synthetic construct
  source 1..87: mol_type = other DNA
aatgatacggcgaccaccgagatctacactctttccctacacgacgctcttccgatctnnncagatcacatctcgtgcgcatctgca

SEQ ID NO: 131 - DNA, 87 bp, synthetic construct
  source 1..87: mol_type = other DNA
aatgatacggcgaccaccgagatctacactctttccctacacgacgctcttccgatctnnnacttgaacatctcgtgcgcatctgca

SEQ ID NO: 132 - DNA, 87 bp, synthetic construct
  source 1..87: mol_type = other DNA
aatgatacggcgaccaccgagatctacactctttccctacacgacgctcttccgatctnnngatcagacatctcgtgcgcatctgca

SEQ ID NO: 133 - DNA, 87 bp, synthetic construct
  source 1..87: mol_type = other DNA
aatgatacggcgaccaccgagatctacactctttccctacacgacgctcttccgatctnnntagcttacatctcgtgcgcatctgca

SEQ ID NO: 134 - DNA, 87 bp, synthetic construct
  source 1..87: mol_type = other DNA
aatgatacggcgaccaccgagatctacactctttccctacacgacgctcttccgatctnnnggctacacatctcgtgcgcatctgca

SEQ ID NO: 135 - DNA, 87 bp, synthetic construct
  source 1..87: mol_type = other DNA
aatgatacggcgaccaccgagatctacactctttccctacacgacgctcttccgatctnnncttgtaacatctcgtgcgcatctgca

SEQ ID NO: 136 - DNA, 87 bp, synthetic construct
  source 1..87: mol_type = other DNA
aatgatacggcgaccaccgagatctacactctttccctacacgacgctcttccgatctnnnagtcaaacatctcgtgcgcatctgca

SEQ ID NO: 137 - DNA, 87 bp, synthetic construct
  source 1..87: mol_type = other DNA
aatgatacggcgaccaccgagatctacactctttccctacacgacgctcttccgatctnnnagttccacatctcgtgcgcatctgca

SEQ ID NO: 138 - DNA, 87 bp, synthetic construct
  source 1..87: mol_type = other DNA
aatgatacggcgaccaccgagatctacactctttccctacacgacgctcttccgatctnnnatgtcaacatctcgtgcgcatctgca

SEQ ID NO: 139 - DNA, 87 bp, synthetic construct
  source 1..87: mol_type = other DNA
aatgatacggcgaccaccgagatctacactctttccctacacgacgctcttccgatctnnnccgtccacatctcgtgcgcatctgca

SEQ ID NO: 140 - DNA, 87 bp, synthetic construct
  source 1..87: mol_type = other DNA
aatgatacggcgaccaccgagatctacactctttccctacacgacgctcttccgatctnnngtagagacatctcgtgcgcatctgca

SEQ ID NO: 141 - DNA, 87 bp, synthetic construct
  source 1..87: mol_type = other DNA
aatgatacggcgaccaccgagatctacactctttccctacacgacgctcttccgatctnnngtccgcacatctcgtgcgcatctgca

SEQ ID NO: 142 - DNA, 83 bp, synthetic construct
  source 1..83: mol_type = other DNA
caagcagaagacggcatacgagatcgtgatgtgactggagttcagacgtgtgctcttccgatccctatcgcggtatcggaatc

(19) United States
(12) Patent Application Publication
(10) Pub. No.: US 2025/0197820 A1
(43) Pub. Date: Jun. 19, 2025

(54) NUCLEIC ACID POLYMERASE AND ITS USE IN PRODUCING NON-DNA NUCLEOTIDE POLYMERS

(71) Applicant: UNITED KINGDOM RESEARCH AND INNOVATION, Wiltshire (GB)

(72) Inventors: Niklas FREUND, Swindon, Wiltshire (GB); Sebastian ARANGUNDY-FRANKLIN, Swindon, Wiltshire (GB); Philipp HOLLIGER, Swindon, Wiltshire (GB)

(21) Appl. No.: 18/690,456

(22) PCT Filed: Sep. 6, 2022
(86) PCT No.: PCT/EP2022/074749

§ 371 Date: Mar. 8, 2024

(30) Foreign Application Priority Data
Sep. 10, 2021 (GB) 2112907.7
May 25, 2022 (GB) 2207699.6

(51) Int. Cl. C12N 9/12; C12P 19/34

(52) U.S. Cl. CPC C12N 9/1252; C12P 19/34; C12Y 207/07007

(57) ABSTRACT

In an aspect, the invention relates to nucleic acid polymerases capable of producing non-DNA polymers. In addition, the invention relates to uses of said polymerases and to the resultant products.

### CROSS-REFERENCE TO RELATED APPLICATIONS

This application is a U.S. National Stage application of International Application No. PCT/EP2022/074749, filed Sep. 6, 2022, which claims the benefit of GB Patent Application No. 2112907.7, filed Sep. 10, 2021, and GB Patent Application No. 2207699.6, filed May 25, 2022, the entire contents of which are hereby incorporated by reference herein in their entireties.

### SEQUENCE LISTING SUBMISSION VIA EFS-WEB

The instant application contains a sequence listing, which has been submitted in XML format via EFS-Web. The contents of the XML copy named “119744-5031_Sequence_Listing”, which was created on May 13, 2024 and is 163,130 bytes in size, the contents of which are incorporated herein by reference in their entirety.

### FIELD OF THE INVENTION

In an aspect, the invention relates to nucleic acid polymerases capable of producing non-DNA polymers. In addition, the invention relates to uses of said polymerases and to the resultant products.

### BACKGROUND OF THE INVENTION

Chemical variations to the canonical (deoxy)ribonucleic acid have gained great interest in the overlapping fields of medicinal chemistry and nucleic acid-based therapeutics (including RNA vaccines), as well as in the synthetic biology of nucleic acids and chemical biology. These modifications encompass a wide range of isomer substitutions, sugar alterations, sugar substituent modifications, nucleobase modifications, including—but not limited to—alteration of the glycosidic linkage, unnatural base-pairing interactions, and modified backbone chemistries. Among these, modifications to the 2-hydroxy group of ribose have been a specific focus.

Such 2′ modifications have been shown to preserve key physicochemical principles of nucleic acid function, such as helical structure and base pairing specificity, while enhancing the biophysical and pharmacological properties of the modified nucleic acids, which has driven their widespread incorporation into nucleic acid therapeutics. Among these, 2′-fluoro (2′F), 2′-O-methyl (2′OMe), 2′-O-(2-methoxyethyl) (MOE), and 2′,4′-locked, -bridged, or -constrained (e.g. tricyclo) nucleic acids have been extensively studied¹.

2′OMe is a naturally-occurring RNA modification found in human rRNA, tRNAs, small nuclear RNA (snRNA) as well as both the Cap- and body of human mRNA and is therefore both inherently biocompatible and unlikely to trigger the innate immune system. Indeed, 2′OMe modifications of viral RNAs appear to be exploited by some viruses as self-signal enabling evasion of interferon-mediated antiviral responses.

The 2′OMe and the related MOE modifications (FIG. 1a, 4a) display a range of favourable physicochemical, pharmacological and immunological properties and their clinical utility has been validated in recently approved nucleic acid drugs such as the silencing RNA (siRNA) drugs Patisiran and Givosiran (2′OMe) and the antisense oligonucleotide (ASO) drugs Nusinersen (Spinraza), Inotersen (Tegsedi) and Volanesorsen (Waylivra) (all MOE)². Furthermore, 2′OMe-RNA modification at purine bases were found to be beneficial in the FDA-approved aptamer drug Pegaptanib (Macugen) for the treatment of age-related macular degeneration.

However, 2′OMe- and MOE-modified oligonucleotides are currently mainly synthesised via solid-phase phosphoramidite-based chemical synthesis, which is limited to short oligomers and a relatively small number of unique sequences and precludes their evolution. Thus, applicable sequences of 2′OMe- and MOE-modified oligonucleotides to be screened for a desired therapeutic effect have to be semi-rationally designed. This approach seems reasonable for ASO therapeutics designed to bind regulatory sequences on messenger RNA, but precludes the de novo discovery and development of aptamer and nucleic acid enzymes therapeutics in these important chemistries as well as hindering the development of nucleic acid nanotechnology objects and devices for both biotechnological and medical applications.

This has spurred the development of a range of engineered polymerases as tools for synthesis and reverse transcription, including mutants of T7 RNA polymerase^{3, 4, 4, 6} or of the Stoffel fragment of Taq DNA polymerase⁷, which have enabled the discovery of partially as well as fully substituted 2′OMe-RNA aptamers^{6,8}. More recently, a mutant of KOD DNA polymerase has been described able to synthesize 1 kb 2′OMe-RNA fragments in the presence of Mn²⁺ ions and enabling the evolution of mixed LNA/2′OMe-RNA aptamers against Thrombin⁹.

Despite these advances, enzymatic synthesis of the bulkier MOE-RNA has not been described. Furthermore, due to the outstanding importance and potential of 2′OMe-RNA, tools for more efficient synthesis of longer or more complex 2′OMe-RNAs remain desirable.

### SUMMARY OF THE INVENTION

In an aspect of the invention, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence is mutated relative to the amino acid sequence of SEQ ID NO: 1 at T541 and/or K592. The amino acid sequence may be mutated relative to the amino acid sequence of SEQ ID NO: 1 at E664.

The amino acid sequence may comprise: i) a T541 mutation and a K592 mutation, ii) a T541 mutation and a E664 mutation, or iii) a T541 mutation, a K592 mutation, and a E664 mutation. The T541 mutation may be T541G, T541S, T541A, T541C, T541D, T541P, or T541N. In a particular embodiment, the T541 mutation is T541G. The K592 mutation may be K592G, K592A, K592C, K592M, K592S, K592D, K592P, K592N, K592T, K592E, K592V, K592Q, K592H, K592I, or K592L. In a particular embodiment, the K592 mutation is K592A or K592G. The E664 mutation may be E664K or E664R.

In a particular embodiment, the amino acid sequence comprises the mutations T541G and K592A.

The amino acid sequence may comprise one or more, or all, of the following mutations: V93Q, D141A, E143A, and A485L relative to SEQ ID NO: 1. The amino acid sequence may comprise one or more, or all, of the following mutations: Y409, I521, and F545 relative to SEQ ID NO: 1. The amino acid sequence may comprise one or more, or all, of the following mutations: Y409G, I521L or I521H, and F545L relative to SEQ ID NO: 1.

The amino acid sequence may comprise a D614 mutation relative to SEQ ID NO: 1. The D614 mutation may be D614N.

The amino acid sequence may have at least 50%, 60%, 70%, 80%, 90%, 95%, 99%, or 100% similarity or identity to the amino acid sequence of SEQ ID NO: 1. The amino acid sequence may have at least 36%, 50%, 60%, 70%, 80%, 90%, 95%, 99%, or 100% similarity or identity to the amino acid sequence of SEQ ID NO: 3 or SEQ ID NO: 4, wherein residues 93, 141, 143, 409, 485, 521, 541, 545, 592, and 664 are invariant. The amino acid sequence may have at least 36%, 50%, 60%, 70%, 80%, 90%, 95%, 99%, or 100% similarity or identity to the amino acid sequence of SEQ ID NO: 5 or SEQ ID NO: 6, wherein residues 93, 141, 143, 409, 485, 521, 541, 545, 592, 614, and 664 are invariant.

The amino acid sequence may comprise SEQ ID NO: 7 or SEQ ID NO: 8.

In another aspect of the invention, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence is mutated relative to the amino acid sequence of SEQ ID NO: 1 at E664R. This nucleic acid polymerase may comprise any features, sequences, mutations, properties, or pattern of mutations as disclosed herein in relation to a nucleic acid polymerase.

The nucleic acid polymerases disclosed herein may comprise an amino acid sequence comprising one or more, or any combination, of the following mutations: D540, D542, K591, K593, Y663, and Q665 relative to SEQ ID NO: 1.

In another aspect of the invention, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence is mutated relative to the amino acid sequence of SEQ ID NO: 1 at one of, all of, or any combination of positions D540, D542, K591, K593, Y663, and/or Q665 relative to SEQ ID NO: 1.

In some embodiments, the mutation at D540 is D540A, D540G, D540S, or D540C. In particular, the mutation may be D540A. In some embodiments, the mutation at D542 is D542A, D542G, D542S, or D542C. In some embodiments, the mutation at K591 is K591G, K591A, K591C, K591M, K591S, K591D, K591P, K591N, K591T, K591E, K591V, K591Q, K591H, K591I, or K591L. In some embodiments, the mutation at K593 is K593G, K593A, K593C, K593M, K593S, K593D, K593P, K593N, K593T, K593E, K593V, K593Q, K593H, K593I, or K593L. In some embodiments, the E663 mutation may be E663K, E663R, or E663H. In some embodiments, the E665 mutation may be E665K, E665R, or E665H.

The nucleic acid polymerases disclosed herein may be capable of producing a non-DNA nucleotide polymer from a nucleic acid template, wherein the non-DNA nucleotide polymer comprises 2′-O-methyl-RNA and (2′OMe-RNA) nucleotides and/or 2′-O-(2-methoxyethyl)-RNA (MOE-RNA) nucleotides.

The nucleic acid polymerases disclosed herein may have an amino acid sequence is derived from the wild type sequence of a nucleic acid polymerase of the polB family. The nucleic acid polymerases disclosed herein may have an amino acid sequence with at least 36% identity to the amino acid sequence of SEQ ID NO: 9.

In another aspect of the invention, there is provided a method for making a non-DNA nucleotide polymer, said method comprising contacting a nucleic acid template with a nucleic acid polymerase of any one of the preceding claims, under conditions conducive to polymerisation. In some embodiments, 2′OMe-RNA nucleotides and/or MOE-RNA nucleotides are provided during the polymerisation, and the resultant non-DNA nucleotide polymer comprises said nucleotides.

In another aspect of the invention, there is provided use of any nucleic acid polymerase disclosed herein for the generation of a non-DNA nucleotide polymer. In some embodiments, the non-DNA nucleotide polymer comprises 2′OMe-RNA nucleosides and/or MOE-RNA nucleosides.

In another aspect of the invention, there is provided a nucleic acid encoding any polymerase disclosed herein.

In another aspect of the invention, there is provided a host cell comprising any polymerase disclosed herein or any nucleic acid encoding a polymerase disclosed herein.

### BRIEF DESCRIPTION OF THE DRAWINGS

FIG. 1 The two-residue steric gate. a) Chemical structure of 2′-O-methyl (2′OMe)-RNA. The 2′-methoxy substituent is highlighted in cyan. b) Sequence alignment showing polymerases Tgo wild type and engineered polymerases and respective key mutations in TGK (blue), TGLLK (green) and 2M (red). The sequences shown in Fig b) are SEQ ID NO: 10, 11, 12, and 13. c) Space-filling model of the ternary structure of KOD DNA polymerase (PDB ID 5OMF) with respective mutations in TGK (blue), TGLLK (green) and 2M (red). d) Structural model of the active site of KOD DNA polymerase (PDB ID 5OMF) with DNA template strand (orange), active site 2′OMe-ATP and 2′OMe-RNA nascent strand (cyan) with 2′-methoxy groups of terminal 3′ and +1 nucleotide shown as space-filling envelope and key steric gate mutations (T541G, K592A) displayed in pink (sticks) with wild-type side-chain residues shown as space-filling envelope highlighting the reduction in steric bulk. e) Denaturing PAGE of 2′OMe-RNA synthesis (DNA primer FD, template TempNpure, full length+72 nt) of steric gate single and double mutations. Note the synergistic effect of T541G and K592A double mutation. f-h) Denaturing PAGE of DNA (H), RNA (OH) and 2′OMe-RNA (OMe) synthesis by TGK, TGLLK or 2M on f) defined-sequence template (DNA/2′OMe-RNA primer FD, template TempNpure, full length+72 nt), g) random N40 template (RNA/2′OMe-RNA primer A-Test2, template Tag3.3-N40-Test2, full length+79 nt), densitometry of N40 synthesis yield: TGLLK 2′OMe-RNA 0%, 2M 2′OMe-RNA 90% (SI FIG. 17), and h) long-range synthesis of a GFP transcript (2′OMe-RNA primer Synth-out1mm, template sfGFP, full length+752 nt).

FIG. 2 Site-specific RNA endonuclease catalysts composed of 2′OMe-RNA. a) Sequence and putative secondary structure of 2′OMezyme R15/5-K selected to target RNA “Sub_KRas_12” [G12D] (residues 213-242 of the human KRAS mRNA bearing the c.35G>A (G12D) mutation) (SEQ ID NOs: 14 and 15) and b) variant 2′OMezyme R15/5-C re-targeted to an alternative RNA “Sub_CTNNB1_33” (residues 85-111 of the human CTNNB1 mRNA bearing the c.98C>A (S33Y) mutation). 2′OMe-RNA nucleotides are shown in cyan or blue (residues changes from R15/5-K to R15/5-C). RNA substrates in orange (KRAS) or red (CTNNB1). Black arrow denotes RNA cleavage site. Circled residues show bases in the “R15_1” parent 2′OMezyme changed during reselection. (below) (SEQ ID NOs: 16 and 17) c, d) (left panel) Urea-PAGE gels show 2′OMezymes (5 μM) performing allele-specific cleavage of substrate RNAs (1 μM) Sub_KRas_12 and Sub_CTNNB1_33 in a bimolecular reaction in trans under quasi-physiological conditions (37° C., pH 7.4, 1 mM Mg2+, 17.5 h). Lane 1 shows partially hydrolyzed RNA substrate. (right panel) Graphs show pre-steady state single turnover reactions with substrate RNAs (1 μM), 2′OMezyme (5 μM) and reaction conditions indicated, at 37° C. Error bars show standard error of the mean (s.e.m.) of three independent replicates. e & f) Reactions between (5 μM) 2′OMezyme and (0.5 μM) synthetic RNA transcripts of e) KRAS (“Sub_KRas_ORF”) and f) CTNNB1 (“Sub_CTNNB1_ORF”) bearing mutations as indicated, under quasi-physiological conditions (37° C., pH 7.4, 1 mM Mg²⁺, 65 h).

FIG. 3 MOE-RNA synthesis a) Chemical structure of 2′-O-(2-methoxyethyl)-RNA (MOE-RNA) with the 2′-O-(2-methoxyethyl) group highlighted. b) Equilibrium of the ribose sugar puckering. The 2′-O-MOE modification shifts the equilibrium towards the C3′-endo (N-type) conformation, comparable to RNA. c) Space-filling representation of the X-ray structure of an MOE-RNA duplex (PDB ID 468D) viewed side (left) and top view (right) with 2′-O-(2-methoxyethyl) groups (highlighted) and overlay of observed 2′-O-(2-methoxyethyl) conformations (stick representation, middle) next to a Newman projection of the ethylene glycol monomethyl ether, which preferentially adopts a gauche conformation respective to the two oxygen atoms. d-f) Denaturing PAGE of 2′OMe-RNA (OMe) and MOE-RNA (MOE) synthesis by TGLLK or 2M on d) defined-sequence template (2′OMe-RNA primer FD, template TempNpure, full length+72 nt), e) random N40 template (2′OMe-RNA primer A-Test2, template Tag3.3-N40-Test2, full length+79 nt), densitometry of N40 synthesis yield: TGLLK 2′OMe-RNA 1%, TGLLK MOE-RNA 0%, 2M 2′OMe-RNA 84%, 2M MOE-RNA 65% (SI FIG. 17), and f) long-range synthesis of a GFP transcript (2′OMe-RNA primer Synth-out1mm, template sfGFP, full length+752 nt).

FIG. 4 2′OMe/MOE-RNA aptamers and binding kinetics. a-c) Sequence and secondary structure representation of anti-VEGF aptamer ARC224⁶ (top panels) with respective SPR sensorgrams and average K_{D} (middle) with residuals of the curves fit (bottom) for a) ARC224 2′OMe-GACU, b) ARC224 2′OMe-GU MOE-AC (MOE substitutions, green) and c) ARC224 2′OMe-U MOE-ACG (SPR binding kinetics: Supplementary Table 3). The sequences are SEQ ID NOs: 18-20.

FIG. 5 Nascent strand steric gate and polymerase motifs. a) Conserved sequence motifs in polB polymerase family showing sequence context and conservation of nascent strand steric gate in motif C (T541) and motif KxY (K592). b) Structural context with active site 2′OMe-ATP (KOD DNA polymerase (PDB ID 5OMF)) showing H-bonding network involving steric gate together with D540 as well as direct contact to +1 minor groove and indirect contact (via H2O) to 3′ end nucleotide. c) Structural conservation of nascent strand steric gate across polB phylogeny from archaeal (left), bacterial (middle) to eukaryotic (right) polB polymerases.

FIG. 6 (Supplementary FIG. 1) Polymerase screen. a) Sequence alignment showing engineered polymerases and respective key mutations in TGLLK (blue and green), TGHLK (orange) and 2M (red). The sequences are SEQ ID NO: 12, 21, and 13. b) Representation of relative location of residues screened (D540, T541, K592, D614, E664) in the polymerase structure (KOD DNA polymerase (PDB ID 5OMF)) using polymerase activity assay (PAA) as described in Materials & Methods. c) Denaturing PAGE of 2′OMe-RNA synthesis by different TGLLK (I521L) single mutants identified in the screen on defined-sequence template (DNA primer FD, template TempNpure, full length+72 nt). Note the positive effect of T541G as well as K592A and E664R mutations. In this context, we also explored mutations to L521H in the TGLLK context that enhanced 2′OMe-RNA synthesis but ultimately favoured the I521L variants. d) Denaturing PAGE of 2′OMe-RNA synthesis by different TGLLK and TGHLK mutants on defined-sequence template (DNA primer FD, template TempNpure, full length+72 nt). Note the synergistic effect of T541G and K592A double mutation. e) Denaturing PAGE of DNA/2′OMe-RNA synthesis by 2M on random N40 template (DNA/2′OMe-RNA primer A-Test2, template Tag3.3-N40-Test2, full length+79 nt).

FIG. 7 (Supplementary FIG. 2). pH and Magnesium dependency of the 2′Omezyme R15/5-K. (a) Normalised activity of 2′Omezyme R15/5-K (5 μM), or an analogous DNAzyme “1023_KrasC” (5 μM), on Sub_Kras_12 [G12D] RNA (1 μM) in varying pH with buffer system as indicated (1 mM Mg²⁺, 37° C. 16.5 h) or (b) concentrations of MgCl₂ (pH 7.4, 37° C., 16.5 h). (c) Pre-steady state single turnover reaction with substrate RNA Sub_Kras_12 [G12D] RNA (1 μM) and 2′Omezyme R15/5-Kras (5 μM) in the absence of Mg²⁺ (pH 7.4, 37° C., 5 mM EDTA). Error bars show standard error of the mean (s.e.m.) of three independent replicates. (d & e) Urea-PAGE gels showing (10 nM) 2′Omezymes (d) R15/5-K or (e) R15/5-C performing multiple turnover catalysis with (1 μM) RNA substrates (d) Sub_KRas_12 [G12D] or (e) Sub_CTNNB1_33 [S33Y], under quasi-physiological conditions (37° C., pH 7.4, 1 mM Mg²⁺).

FIG. 8 (Supplementary FIG. 3). Characterisation of the 2′OMezyme R15/5-K-catalysed RNA cleavage product. (a) MALDI-ToF spectrum of 5′ RNA product of R15/5-K catalysed cleavage of RNA Sub_KRas_12 [G12D]. Expected masses for the product are shown with a 3′ monophosphate (p) or cyclic phosphate (>p) (depicted in schematic) (SEQ ID NO: 22). (b) Phosphatase assay of 5′ product of R15/5-K-catalysed cleavage of RNA Sub_KRas_12 [G12D]. Urea-PAGE gel showing PAGE-purified 5′ product RNA treated with bovine intestinal phosphatase (CIP; removes 2′- or 3′-terminal monophosphate, but not 2′,3′-cyclic phosphate), or T4 polynucleotide kinase (T4 PNK; removes both mono- and 2′,3′-cyclic phosphate), with or without prior acid hydrolysis. Lane 1 shows partially hydrolysed RNA substrate as a marker.

FIG. 9 (Supplementary FIG. 4). Serum nuclease resistance of the 2′OMezyme R15/5-K. (a) Urea-PAGE gel and graph showing stability of 2′OMezyme R15/5-K and an analogous DNAzyme “1023_KRasC” in 90% human serum at 37° C. (b) Urea-PAGE gel showing activity of (5 μm) 2′OMezyme R15/5-K, before (lane 3) or after (lane 4) incubation in 90% human serum at 37° C. for 120 h, by reaction with RNA substrate Sub_KRas_12 [G12D] (1 μM) under quasi-physiological conditions (pH 7.4, 1 mM Mg²⁺, 37° C. 18 h). Lane 1 shows partially hydrolysed RNA substrate as a marker.

FIG. 10 (Supplementary FIG. 5). Mutation screen of putative unpaired substrate-proximal nucleobases in the re-targeted 2′OMezyme R15/5-CTNNB1. (a) Sequence and putative secondary structure of retargeted 2′OMezyme “R15/5-CTNNB1” bound to its RNA substrate “Sub_CTNNB1_33” (residues 85-111 of the human CTNNB1 mRNA bearing the c.98C>A (S33Y) mutation). 2′OMe-RNA nucleotides are shown in cyan or blue (sequence changes from R15/5-K) or orange (indicates changes from parent R15/5_1 2′OMezyme), RNA in orange. Black arrow denotes RNA cleavage site. Variants of the 2′OMezyme were prepared with all possible single mutations (or one double mutation, A39G+U45A) of putative unpaired positions adjacent to the substrate-binding arms as indicated by circles. The sequences shown are SEQ ID NO: 16 and 23. (b) Urea-PAGE gel showing activity of variants of R15/5-CTNNB1 (2.5 μM) on RNA substrate Sub_CTNNB1_33 [S33Y] (1 μM) under quasi-physiological conditions (pH 7.4, 1 mM Mg²⁺, 37° C., 24 h). The R15/5-CTNNB1: A39G, U45A variant *called R15/5-C) was used for all other experiments.

FIG. 11 (Supplementary FIG. 6): General synthesis route for the triphosphorylation of 2′-O-(2-methoxyethyl)ribonucleosides. Base=adenine (A, compound a), 5-methyluracil (m⁵U, compound b), guanine (G, compound c), or cytosine (C, compound d). i) POCl₃, proton sponge, (MeO)₃PO, −15° C.; ii) (Bu₄N)₃HP₂O₇, Bu₃N, DMF, RT, 30 min; iii) TEAB buffer, RT, 13-28% over three steps (one-pot).

FIG. 12 (Supplementary FIG. 7) Time course of 2′OMe-RNA and MOE-RNA synthesis. a) Denaturing PAGE of time course of 2′OMe-RNA and MOE-RNA synthesis by TGLLK and 2M on defined-sequence template (2′OMe-RNA primer FD, template TempNpure, full length+72 nt). 2M reaches full length synthesis (+72 nt) in <5 min (2′OMe-RNA), respectively <20 min (MOE-RNA). b) Denaturing PAGE of time course of DNA, 2′OMe-RNA, and MOE-RNA synthesis by 2M on random N40 sequence template (2′OMe-RNA primer FD-Test2, template Tag3.3-N40-Test2, full length+79 nt). 2M reaches full length synthesis (+79 nt) in <1 min (DNA), <10 min (2′OMe-RNA), respectively <30 min (MOE-RNA, densitometry measurements in SI FIG. 17).

FIG. 13 (Supplementary FIG. 8) Synthesis of 2′OMe-RNA, mixed 2′OMe/MOE-RNA, and all-MOE-RNA. Denaturing PAGE of (left to right) 2′OMe-RNA synthesis, mixed 2′OMe/MOE-RNA synthesis (2′OMe-U/G/C MOE-A, 2′OMe-G/C MOE-A/m⁵U, 2′OMe-C MOE-A/m⁵U/G), and all-MOE-RNA synthesis by TGLLK and 2M on defined sequence template (2′OMe-RNA primer FD, template TempN, full length+57 nt). Note the increasing gel shift (retardation) with increasing MOE content illustrating the increasing hydrodynamic envelope of 2′-O-(2-methoxyethyl) groups protruding from the helix.

FIG. 14 (Supplementary FIG. 9) 2′OMe/MOE-RNA aptamers. a), b) Sequence and secondary structure representation of anti-VEGF aptamer ARC224¹³ (top panels) with respective SPR sensorgrams and average K_{D} (middle) with residuals of the curves fit (bottom) for a) ARC224 2′OMe- and ARC224 2′OMe m⁵U and b) ARC224 MOE. Note the reduced affinity of ARC224 2′OMe-m⁵U compared to ARC224 (2′OMe-U). The sequences are SEQ ID NO: 24 and 25.

FIG. 15. (Supplementary FIG. 10) Polymerase phylogeny and motif conservation. a) Phylogenetic tree of polB-family polymerases including archaeal (Pyrococcales/Thermococcales), bacterial (E. coli, RB69 bacteriophage), eukaryotic (Saccharomyces), mammalian, (human), and viral (Vaccinia) polymerases. b) Sequence alignment and conservation of motifs C (left) and KxY (right) across different polB polymerases. The sequences are SEQ ID NOs: 26-40.

FIG. 16 (Supplementary FIG. 11) Fidelity of MOE-RNA synthesis by 2M. Dropout assay of MOE-RNA fidelity showing templated synthesis of first four bases on TempNpure template (3′-CTAG-5′ after priming site) with one MOE-NTP omitted (left to right: MOE-CTP, MOE-GTP, MOE-m5UTP, MOE-ATP) showing expected stalling pattern for correct incorporation except for MOE-GTP, indicating some misincorporation opposite template C. Also shown is full length synthesis (+72 nt) with all MOE-NTPs.

FIG. 17 (Supplementary FIG. 12) Steady-state kinetics for extension of a 2′OMe-RNA primer with ATP, 2′OMe-ATP and MOE-ATP by 2M. a) Steady-state kinetic parameter V₀ (μmole/min) 718 plotted against nucleotide triphosphate concentration [NTP] for extension of 2′OMe-RNA primer FAM-FD on template BFL770 (Supplementary Table 1) for ATP (black circles), 2′OMe-ATP (red squares) and MOE-ATP (cyan triangles) by 2M (n=3). b) Table of steady-state kinetic parameters for single nucleotide incorporation by 2M.

FIG. 18 (Supplementary FIG. 13) Benchmarking 2M against other polymerases. a) Denaturing PAGE of RNA, 2′F-DNA, and 2′OMe-RNA synthesis by 2M and engineered Taq Stoffel fragment variant SFM4-6 on defined-sequence template (DNA or 2′OMe-RNA primer FD, template TempNpure, full length+72 nt) under optimal conditions for each polymerase. b) Denaturing PAGE of RNA and 2′OMe-RNA transcription by T7 RNA polymerase (WT) and engineered T7 RNAP variant RGVG-M6 on a long defined-sequence template (generated as described in Materials & Methods, 901 bp) under optimal conditions for RGVG-M6. c) Denaturing PAGE of 2′OMe-RNA primer extension synthesis and transcription by 2M and engineered T7 RNAP variant RGVG-M6 in the presence and absence of 1.5 mM Mn²⁺ on a long defined-sequence template (for transcription reaction: template generated as described in Materials & Methods, 901 bp; for primer extension reaction: 2′OMe-RNA primer Synthout1mm, template sfGFP, full length+752 nt) under equimolar nucleic acid input (50 nM (0.5 pmol) input of primer and dsDNA template).

FIG. 19 (Supplementary FIG. 14) Polymerase comparison. Denaturing PAGE of 2′OMe- and MOE-RNA synthesis by 2M, engineered KOD variant DGLNK¹⁴, and 2M bearing the DGLNK mutation D614N (2M D614N) on defined-sequence template (2′OMe-RNA primer FD, template TempNpure, full length+72 nt) under optimal conditions for each polymerase and both in the presence and absence of Mn²⁺ ions. As described¹⁴, KOD DGLNK performs best in 2′OMe-RNA synthesis in the presence of Mn²⁺ but is unable to synthesize MOE-RNA efficiently. Interestingly, the D614N mutation confers a small increase in activity to 2M in the context of 2′OMe-RNA synthesis.

FIG. 20 (Supplementary FIG. 15) Polymerase comparison 2M vs 3M. a) Sequence alignment showing polymerases Tgo wild-type and engineered polymerases and respective key mutations in TGK (blue), TGLLK (green) and 2M (red) and 3M (taupe). The sequences are SEQ ID NOs: 10, 11, 12, 13, 41. b) Denaturing PAGE of DNA (H), RNA (OH), 2′F-RNA (F), and 2′OMe-RNA (OMe) synthesis by TGK, TGLLK, 2M and 3M on defined-sequence template (DNA/2′OMe-RNA primer FD, template TempNpure, full length+72 nt), c) Denaturing PAGE of 2′OMe-RNA (OMe) and MOE-RNA (MOE) synthesis by TGLLK, 2M and 3M on defined sequence template (2′OMe-RNA primer FD, template TempNpure, full length+72 nt).

FIG. 21 (Supplementary FIG. 16) 2′OMezyme R15/5-C as an analogue of the hairpin ribozyme. a) Sequence and putative secondary structure of 2′OMezyme R15/5-C engineered to target the human CTNNB1 mRNA RNA (top) and the Hairpin ribozyme (Hpz) (bottom). 2′OMe-RNA nucleotides (R15/5-C) are shown in orange or cyan (mutations either identical or mutated to Hpz consensus). RNA nucleotides are shown in red or cyan (if equivalent to R15/5-C). RNA substrates are shown in grey. Black arrow denotes RNA cleavage Site. The sequences are SEQ ID NOs: 42, 43, 44. b) Urea-PAGE gels showing cleavage of Sub_CTNNB1_33 substrate RNA (1 μM) by variants of R15/5-C with mutations towards Hpz consensus. (c) Urea-PAGE gel showing RNA ligation activity of 2′OMezyme R15/5_1. PAGE-purified 5′ (FITC-labelled) and 3′ (unlabelled) RNA cleavage products of R15/5-K-catalysed cleavage of Sub_KRas_12 [G12D] (1 μM each) re-incubated with R15/5-K (5 μM) at −7° C. in ice (lanes 2-5) or supercooled (lanes 7-10), or at 37° C., in quasi-physiological buffer (pH 7.4, 1 mM Mg²⁺) (lanes 4, 5, 9, 10, 12 and 13) or magnesium-free buffer (pH 7.4, 5 mM EDTA) (lanes 2, 3, 7, 8) for 20 h. Lane 1 shows partially hydrolysed RNA Sub_KRas_12 [G12D] substrate as a marker.

FIG. 22 (Supplementary FIG. 17) Densitometry measurements of a) DNA, 2′OMe-RNA and MOE-RNA synthesis time course by 2M on an N40 library (SI FIG. 7b) and b) 2′OMe-RNA and MOE-RNA synthesis yield by TGLLK and 2M on an N40 library (FIGS. 1g and 3e).

### DETAILED DESCRIPTION

Provided herein are polymerases that may contain mutations in a two-residue steric control “gate”. Polymerases provided herein have been engineered to reduce the steric bulk of this gate, and the polymerases have increased capacity to synthesise xeno nucleic acid (XNA) polymers. In particular, the polymerases may be capable of incorporating 2′-O-methyl-RNA and (2′OMe-RNA) nucleotides and/or 2′-O-(2-methoxyethyl)-RNA (MOE-RNA) nucleotides into a polymer.

Thus, in an aspect, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence is mutated relative to the amino acid sequence of SEQ ID NO: 1 at T541 and/or K592. In other words, the polymerase may be mutated relative to the amino acid sequence of SEQ ID NO: 1 at i) T541, ii) K592, or iii) T541 and K592.

The polymerase may comprise an E664 mutation relative to SEQ ID NO: 1.

In some embodiments, the nucleic acid polymerase comprises a mutation at T541 and at K592. In some embodiments, the nucleic acid polymerase comprises a mutation at T541 and at E664. In some embodiments, the nucleic acid polymerase comprises a mutation at T541, K592, and E664.

The mutations at T541 and/or K592 may be to any less bulky residue. Thus, the mutations may be to any residue that presents less of a steric block than threonine at position 541 or lysine at position 592. The T541 mutation may be selected from the group T541G, T541S, T541A, T541C, T541D, T541P, or T541N. In particular, the T541 mutation may be T541G or T541S. The K592 mutation may be K592G, K592A, K592C, K592M, K592S, K592D, K592P, K592N, K592T, K592E, K592V, K592Q, K592H, K592I, or K592L. In particular, the K592 mutation may be K592G, K592A, K592C, or K592M.

The mutation at E664 may be to any positively charged residue. The E664 mutation may be E664K, E664R, or E664H. In particular, the E644 mutation may be E664K or E664R.

In an embodiment, the mutation at T541 is T541G. In an embodiment, the mutation at K592 is K592A or K592G. In an embodiment, the mutation at E644 is E664K or E664R. The polymerase may comprise the mutations T541G and K592A. The polymerase may comprise the mutations T541G and E664K. The polymerase may comprise the mutations T541G and E664R. The polymerase may comprise the mutations T541G, K592A, and E664K. The polymerase may comprise the mutations T541G, K592A, and E664R.

The polymerase may comprise the mutation T541G and a mutation at position K592. The mutation at position K592 may be any disclosed herein, such as A or G. The polymerase may comprise the mutation T541G, a mutation at position K592, and a mutation at position E664.

The polymerase may contain mutations at any one of, all of, or any combination of positions D540, D542, K591, K593, Y663, and/or Q665 relative to SEQ ID NO: 1. In some examples, the mutations at positions D540, D542, K591, and/or K593 are to any less bulky residue, i.e. any residue that presents less of a steric block than the wild type residue. In some examples, the mutations at positions Y663, and/or Q665 are to any positively charged residue.

In some embodiments, the mutation at D540 is D540A, D540G, D540S, or D540C. In particular, the mutation may be D540A.

In some embodiments, the mutation at D542 is D542A, D542G, D542S, or D542C.

In some embodiments, the mutation at K591 is K591G, K591A, K591C, K591M, K591S, K591D, K591P, K591N, K591T, K591E, K591V, K591Q, K591H, K591I, or K591L.

In some embodiments, the mutation at K593 is K593G, K593A, K593C, K593M, K593S, K593D, K593P, K593N, K593T, K593E, K593V, K593Q, K593H, K593I, or K593L.

In some embodiments, the E663 mutation may be E663K, E663R, or E663H.

In some embodiments, the E665 mutation may be E665K, E665R, or E665H.

In a particular embodiment, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence is mutated relative to the amino acid sequence of SEQ ID NO: 1 at T541 and K592. In an embodiment, the nucleic acid polymerase comprises the mutations T541G and K592A/K592G. In a certain embodiment, the nucleic acid polymerase comprises the mutations T541G and K592A.

In an another embodiment, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence is mutated relative to the amino acid sequence of SEQ ID NO: 1 at T541 and K592, for instance T541G and K592A/K592G, and wherein the amino acid sequence is mutated relative to the amino acid sequence of SEQ ID NO: 1 at one or more, or any combination, of positions D540, D542, K591, K593, Y663, and/or Q665 relative to SEQ ID NO: 1.

In another embodiment, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence is mutated relative to the amino acid sequence of SEQ ID NO: 1 at T541, K592, and E644. In an embodiment, the nucleic acid polymerase comprises the mutations T541G, K592A/K592G, and E664K/E664R. In a certain embodiment, the nucleic acid polymerase comprises the mutations T541G, K592A, and E664K. In another embodiment, the nucleic acid polymerase comprises the mutations T541G, K592A, and E664R.

In another embodiment, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence is mutated relative to the amino acid sequence of SEQ ID NO: 1 at T541, K592, and E644, for instance T541G, K592A/K592G, and E664K/E664R, and wherein the amino acid sequence is mutated relative to the amino acid sequence of SEQ ID NO: 1 at one or more, or any combination, of positions D540, D542, K591, K593, Y663, and/or Q665 relative to SEQ ID NO: 1.

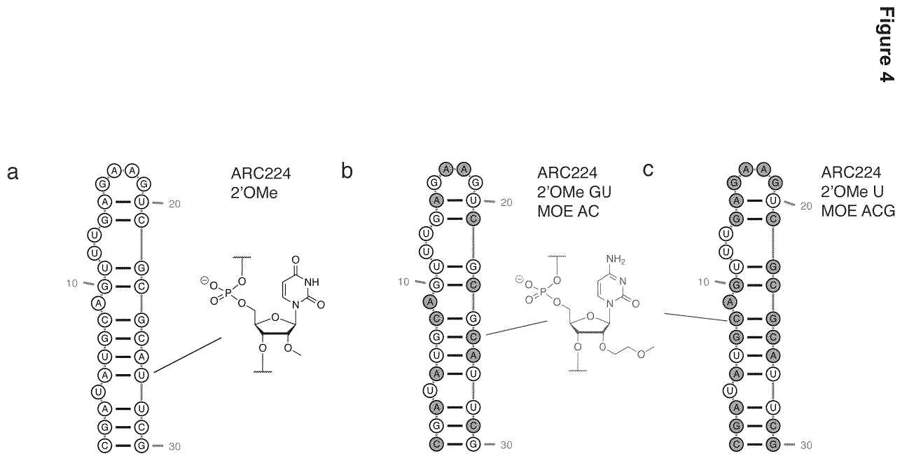

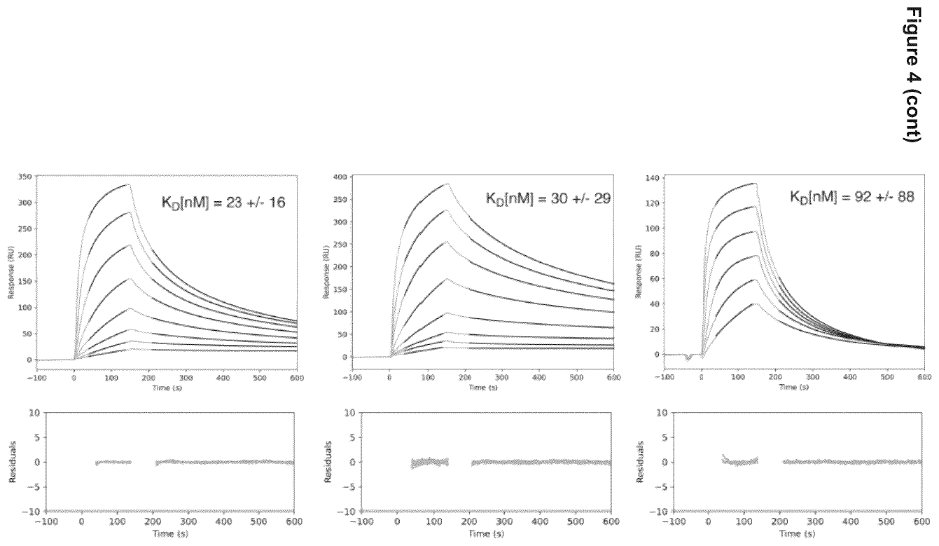

Both T541 and K592 are part of motifs (motif C and KxY, respectively) that are very highly conserved both at the sequence and at the structural level (FIG. 5, SI FIG. 10) in polB polymerases of archaeal, eukaryotic, and even viral origin (Kazlauskas et al. Diversity and evolution of B-family DNA polymerases. Nucleic Acids Res 2020, 48(18): 620 10142-10156). Thus, the mutations of the present disclosure may be applied to the polymerase sequence of, or derived from, any polymerase from the polB family. In particular embodiments, the backbone is any polB polymerase. In other embodiments, the backbone is any polB polymerase excluding viral polymerases. The backbone may be of a polymerase from the Archaeal Thermococcus and/or Pyrococcus genera.

The polymerase may be a variant of the polymerase from T. gorgonarius (Tgo). The sequence of wild type Tgo is shown below:

(SEQ ID NO: 1)
MILDTDYITEDGKPVIRIFKKENGEFKIDYDRNFEPYIYALLKDDSAIEDVKKITAERHGTTVRVV
RAEKVKKKFLGRPIEVWKLYFTHPQDVPAIRDKIKEHPAVVDIYEYDIPFAKRYLIDKGLIPMEGD
EELKMLAFDIETLYHEGEEFAEGPILMISYADEEGARVITWKNIDLPYVDVVSTEKEMIKRFLKVV
KEKDPDVLITYNGDNFDFAYLKKRSEKLGVKFILGREGSEPKIQRMGDRFAVEVKGRIHFDLYPVI
RRTINLPTYTLEAVYEAIFGQPKEKVYAEEIAQAWETGEGLERVARYSMEDAKVTYELGKEFFPME
AQLSRLVGQSLWDVSRSSTGNLVEWFLLRKAYERNELAPNKPDERELARRRESYAGGYVKEPERGL
WENIVYLDERSLYPSIIITHNVSPDTLNREGCEEYDVAPQVGHKFCKDFPGFIPSLLGDLLEERQK
VKKKMKATIDPIEKKLLDYRQRAIKILANSFYGYYGYAKARWYCKECAESVTAWGRQYIETTIREI
EEKFGFKVLYADTDGFFATIPGADAETVKKKAKEFLDYINAKLPGLLELEYEGFYKRGFFVTKKKY
AVIDEEDKITTRGLEIVRRDWSEIAKETQARVLEAILKHGDVEEAVRIVKEVTEKLSKYEVPPEKL
VIYEQITRDLKDYKATGPHVAVAKRLAARGIKIRPGTVISYIVLKGSGRIGDRAIPFDEFDPAKHK
YDAEYYIENQVLPAVERILRAFGYRKEDLRYQKTRQVGLGAWLKPKT

Any nucleic acid polymerase disclosed herein may comprise an amino acid sequence having at least 36%, 50%, 60%, 70%, 80%, 90%, 95%, 99%, or 100% similarity or identity to the amino acid sequence of SEQ ID NO: 1. Said amino acid sequence may have at least 80%, 90% 95%, 99%, or 100% similarity or identity to the amino acid sequence of SEQ ID NO: 1. Said amino acid sequence may be mutated relative to the amino acid sequence of SEQ ID NO: 1 at T541 and/or K592, and optionally E664. The polymerase may include any specific mutations or pattern of mutations as disclosed herein.

The polymerases disclosed herein may comprise a V93 mutation relative to SEQ ID NO: 1. The mutation may be V93Q.

The polymerases disclosed herein may comprise a D141 mutation and/or a E143 mutation relative to SEQ ID NO: 1. The mutations may be D141A and/or E143A.

The polymerases disclosed herein may comprise a A485 mutation relative to SEQ ID NO: 1. The mutation may be A485L.

The amino acid sequence of the nucleic acid polymerase may further comprise one or more, or all, of the following mutations: V93Q, D141A, E143A, and A485L.

V93Q is a mutation known to disable uracil-stalling, D141A and E143A reduce 3′-5′ exonuclease function, and the “Therminator” mutation (A485L) is known to enhance the incorporation of unnatural substrates. The sequence of the Tgo polymerase comprising these mutations (henceforth termed TgoT) is shown below:

(SEQ ID NO: 2)
MILDTDYITEDGKPVIRIFKKENGEFKIDYDRNFEPYIYALLKDDSAIEDVKKITAERHGTTVRVV
RAEKVKKKFLGRPIEVWKLYFTHPQDQPAIRDKIKEHPAVVDIYEYDIPFAKRYLIDKGLIPMEGD
EELKMLAFAIATLYHEGEEFAEGPILMISYADEEGARVITWKNIDLPYVDVVSTEKEMIKRFLKVV
KEKDPDVLITYNGDNFDFAYLKKRSEKLGVKFILGREGSEPKIQRMGDRFAVEVKGRIHFDLYPVI
RRTINLPTYTLEAVYEAIFGQPKEKVYAEEIAQAWETGEGLERVARYSMEDAKVTYELGKEFFPME
AQLSRLVGQSLWDVSRSSTGNLVEWFLLRKAYERNELAPNKPDERELARRRESYAGGYVKEPERGL
WENIVYLDFRSLYPSIIITHNVSPDTLNREGCEEYDVAPQVGHKFCKDFPGFIPSLLGDLLEERQK
VKKKMKATIDPIEKKLLDYRQRLIKILANSFYGYYGYAKARWYCKECAESVTAWGRQYIETTIREI
EEKFGFKVLYADTDGFFATIPGADAETVKKKAKEFLDYINAKLPGLLELEYEGFYKRGFFVTKKKY
AVIDEEDKITTRGLEIVRRDWSEIAKETQARVLEAILKHGDVEEAVRIVKEVTEKLSKYEVPPEKL
VIYEQITRDLKDYKATGPHVAVAKRLAARGIKIRPGTVISYIVLKGSGRIGDRAIPFDEFDPAKHK
YDAEYYIENQVLPAVERILRAFGYRKEDLRYQKTRQVGLGAWLKPKT

The mutations of any of the embodiments disclosed herein wherein the mutations are applied to a backbone comprising SEQ ID NO: 1 may be applied to a backbone comprising SEQ ID NO: 2, wherein residues 93, 141, 143, and 485 are invariant. For instance, in some embodiments, there is provided a nucleic acid polymerase capable of producing a non-DNA nucleotide polymer from a nucleic acid template, said polymerase comprising amino acid sequence having at least 36%, 50%, 60%, 70%, 80%, 90%, 95%, 99%, or 100% similarity or identity to the amino acid sequence of SEQ ID NO: 2, wherein said amino acid sequence is mutated relative to the amino acid sequence of SEQ ID NO: 1 at T541 and/or K592, and optionally E664, and wherein residues 93, 141, 143, and 485 are invariant. The amino acid sequence may also comprise mutations at any one of, or any combination of, positions D540, D542, K591, K593, Y663, and/or Q665.

The polymerases disclosed herein may comprise a Y409 mutation relative to SEQ ID NO: 1. In some examples, the Y409 mutation may be Y409N or Y409G.

The polymerases disclosed herein may comprise a I521 mutation relative to SEQ ID NO: 1.

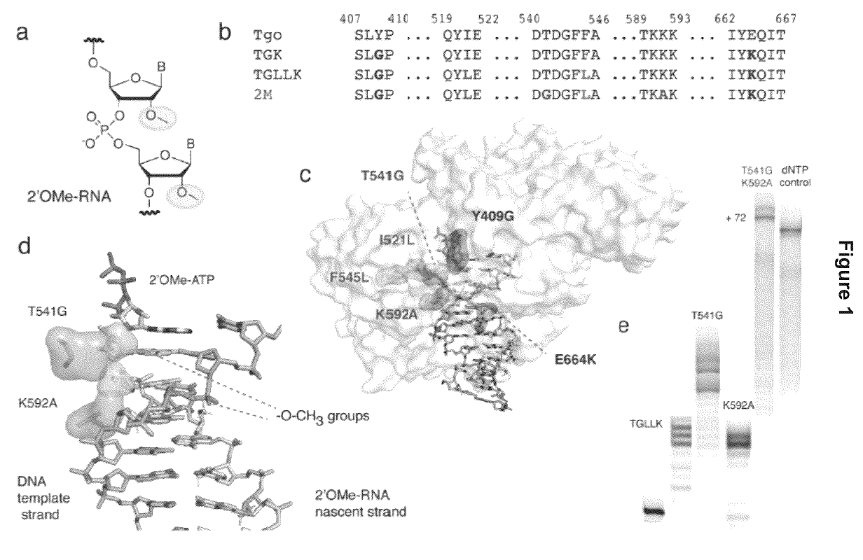

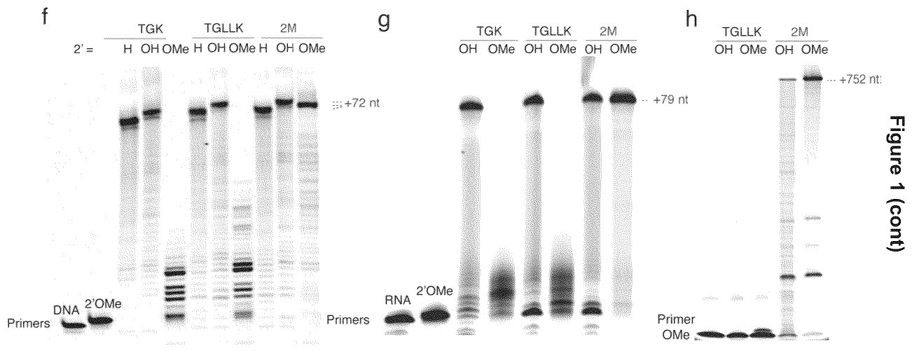

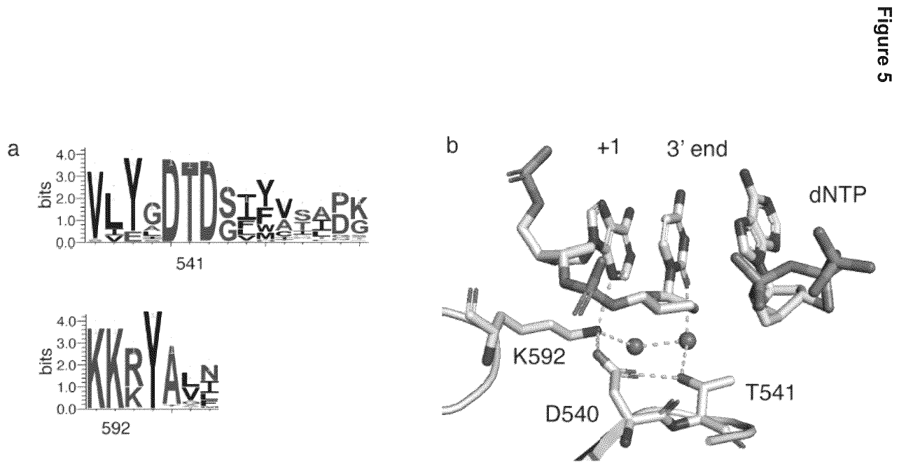

In some examples, the I521 mutation may be I521L or I521H (see FIG. 6 (Supp. FIG. 1)).

The polymerases disclosed herein may comprise a F545 mutation relative to SEQ ID NO: 1. In some examples, the F545 mutation may be F545L.

The polymerases disclosed herein may comprise a D614 mutation relative to SEQ ID NO: 1. In some examples, the D614 mutation may be D614N (see FIG. 19 (Supp. FIG. 14)).

The polymerase may comprise mutations Y409, I521, T541G, F545, K592A/K592G, and E664 relative to SEQ ID NO: 1 or SEQ ID NO: 2. The polymerase may comprise the mutations Y409G/Y409N, I521L/I521H, T541, F545L, K592, and E664K/E664R relative to SEQ ID NO: 1 or SEQ ID NO: 2. The polymerase may comprise the mutations Y409G/Y409N, I521L/I521H, T541G, F545L, K592A/K592G, and E664K/E664R relative to SEQ ID NO: 1 or SEQ ID NO: 2. The polymerase may comprise the mutations Y409G, I521L, T541G, F545L, K592A, and E664K relative to SEQ ID NO: 1 or SEQ ID NO: 2. The polymerase may comprise the mutations V93Q, D141A, E143A, Y409G, A485L, I521L, T541G, F545L, K592A, and E664K relative to SEQ ID NO: 1. The polymerase may comprise the mutations Y409G, I521L, T541G, F545L, K592A, and E664R relative to SEQ ID NO: 1 or SEQ ID NO: 2. The polymerase may comprise the mutations V93Q, D141A, E143A, Y409G, A485L, I521L, T541G, F545L, K592A, and E664R relative to SEQ ID NO: 1.

The polymerase may comprise mutations Y409, I521, T541G, F545, K592A/K592G, D614N, and E664 relative to SEQ ID NO: 1 or SEQ ID NO: 2. The polymerase may comprise the mutations Y409G/Y409N, I521L/I521H, T541, F545L, K592, D614, and E664K/E664R relative to SEQ ID NO: 1 or SEQ ID NO: 2. The polymerase may comprise the mutations Y409G/Y409N, I521L/I521H, T541G, F545L, K592A/K592G, D614N, and E664K/E664R relative to SEQ ID NO: 1 or SEQ ID NO: 2. The polymerase may comprise the mutations Y409G, I521L, T541G, F545L, K592A, D614N, and E664K relative to SEQ ID NO: 1 or SEQ ID NO: 2. The polymerase may comprise the mutations V93Q, D141A, E143A, Y409G, A485L, I521L, T541G, F545L, K592A, D614N, and E664K relative to SEQ ID NO: 1. The polymerase may comprise the mutations Y409G, I521L, T541G, F545L, K592A, D614N, and E664R relative to SEQ ID NO: 1 or SEQ ID NO: 2. The polymerase may comprise the mutations V93Q, D141A, E143A, Y409G, A485L, I521L, T541G, F545L, K592A, D614N, and E664R relative to SEQ ID NO: 1.

In a particular embodiment, the nucleic acid polymerase may comprise or may be of the following amino acid sequence:

MILDTDYITEDGKPVIRIFKKENGEFKIDYDRNFEPYIYALLKDDSAIEDVKKITAERHGTTVRVV
RAEKVKKKFLGRPIEVWKLYFTHPQDQPAIRDKIKEHPAVVDIYEYDIPFAKRYLIDKGLIPMEGD
EELKMLAFAIATLYHEGEEFAEGPILMISYADEEGARVITWKNIDLPYVDVVSTEKEMIKRFLKVV
KEKDPDVLITYNGDNFDFAYLKKRSEKLGVKFILGREGSEPKIQRMGDRFAVEVKGRIHFDLYPVI
RRTINLPTYTLEAVYEAIFGQPKEKVYAEEIAQAWETGEGLERVARYSMEDAKVTYELGKEFFPME
AQLSRLVGQSLWDVSRSSTGNLVEWFLLRKAYERNELAPNKPDERELARRRESYAGGYVKEPERGL
WENIVYLDERSLGPSIIITHNVSPDTLNREGCEEYDVAPQVGHKFCKDFPGFIPSLLGDLLEERQK
VKKKMKATIDPIEKKLLDYRQRLIKILANSFYGYYGYAKARWYCKECAESVTAWGRQYLETTIREI
EEKFGFKVLYADGDGFLATIPGADAETVKKKAKEFLDYINAKLPGLLELEYEGFYKRGFFVTKAKY
AVIDEEDKITTRGLEIVRRDWSEIAKETQARVLEAILKHGDVEEAVRIVKEVTEKLSKYEVPPEKL
VIYKQITRDLKDYKATGPHVAVAKRLAARGIKIRPGTVISYIVLKGSGRIGDRAIPFDEFDPAKHK
YDAEYYIENQVLPAVERILRAFGYRKEDERYQKTRQVGLGAWLKPKT (SEQ ID NO: 3; also
known as 2M polymerase).

Thus, in an aspect, there is provided a polymerase of an amino acid sequence having at least 36%, 50%, 60%, 70%, 80%, 90%, 95%, 99%, or 100% similarity or identity to the amino acid sequence of SEQ ID NO: 3, wherein residues 93, 141, 143, 409, 485, 521, 541, 545, 592, and 664 are invariant (i.e. the mutations V93Q, D141A, E143A, Y409G, A485L, I521L, T541G, F545L, K592A, and E664K, are maintained).

In a particular embodiment, the nucleic acid polymerase may comprise or may be of the following amino acid sequence:

MILDTDYITEDGKPVIRIFKKENGEFKIDYDRNFEPYIYALLKDDSAIEDVKKITAERHGTTVRVV
RAEKVKKKFLGRPIEVWKLYFTHPQDQPAIRDKIKEHPAVVDIYEYDIPFAKRYLIDKGLIPMEGD
EELKMLAFAIATLYHEGEEFAEGPILMISYADEEGARVITWKNIDLPYVDVVSTEKEMIKRFLKVV
KEKDPDVLITYNGDNFDFAYLKKRSEKLGVKFILGREGSEPKIQRMGDRFAVEVKGRIHFDLYPVI
RRTINLPTYTLEAVYEAIFGQPKEKVYAEEIAQAWETGEGLERVARYSMEDAKVTYELGKEFFPME
AQLSRLVGQSLWDVSRSSTGNLVEWFLLRKAYERNELAPNKPDERELARRRESYAGGYVKEPERGL
WENIVYLDERSLGPSIIITHNVSPDTLNREGCEEYDVAPQVGHKFCKDFPGFIPSLLGDLLEERQK
VKKKMKATIDPIEKKLLDYRQRLIKILANSFYGYYGYAKARWYCKECAESVTAWGRQYLETTIREI
EEKFGFKVLYADGDGFLATIPGADAETVKKKAKEFLDYINAKLPGLLELEYEGFYKRGFFVTKAKY
AVIDEEDKITTRGLEIVRRDWSEIAKETQARVLEAILKAGDVEEAVRIVKEVTEKLSKYEVPPEKL
VIYRQITRDLKDYKATGPHVAVAKRLAARGIKIRPGTVISYIVLKGSGRIGDRAIPFDEFDPAKHK
YDAEYYIENQVLPAVERILRAFGYRKEDLRYQKTRQVGLGAWLKPKT (SEQ ID NO: 4; also
known as 3M polymerase).

Thus, in an aspect, there is provided a polymerase of an amino acid sequence having at least 36%, 50%, 60%, 70%, 80%, 90%, 95%, 99%, or 100% similarity or identity to the amino acid sequence of SEQ ID NO: 4, wherein residues 93, 141, 143, 409, 485, 521, 541, 545, 592, and 664 are invariant (i.e. the mutations V93Q, D141A, E143A, Y409G, A485L, I521L, T541G, F545L, K592A, and E664R, are maintained).

In a particular embodiment, the nucleic acid polymerase may comprise or may be of the following amino acid sequence:

MILDTDYITEDGKPVIRIFKKENGEFKIDYDRNFEPYIYALLKDDSAIEDVKKITAERHGTTVRVV
RAEKVKKKFLGRPIEVWKLYFTHPQDQPAIRDKIKEHPAVVDIYEYDIPFAKRYLIDKGLIPMEGD
EELKMLAFAIATLYHEGEEFAEGPILMISYADEEGARVITWKNIDLPYVDVVSTEKEMIKRFLKVV
KEKDPDVLITYNGDNFDFAYLKKRSEKLGVKFILGREGSEPKIQRMGDRFAVEVKGRIHFDLYPVI
RRTINLPTYTLEAVYEAIFGQPKEKVYAEEIAQAWETGEGLERVARYSMEDAKVTYELGKEFFPME
AQLSRLVGQSLWDVSRSSTGNLVEWFLLRKAYERNELAPNKPDERELARRRESYAGGYVKEPERGL
WENIVYLDFRSLGPSIIITHNVSPDTLNREGCEEYDVAPQVGHKFCKDFPGFIPSLLGDLLEERQK
VKKKMKATIDPIEKKLLDYRQRLIKILANSFYGYYGYAKARWYCKECAESVTAWGRQYLETTIREI
EEKFGFKVLYADGDGFLATIPGADAETVKKKAKEFLDYINAKLPGLLELEYEGFYKRGFFVTKAKY
AVIDEEDKITTRGLEIVRRNWSEIAKETQARVLEAILKHGDVEEAVRIVKEVTEKLSKYEVPPEKL
VIYKQITRDLKDYKATGPHVAVAKRLAARGIKIRPGTVISYIVLKGSGRIGDRAIPFDEFDPAKHK
YDAEYYIENQVLPAVERILRAFGYRKEDLRYQKTRQVGLGAWLKPKT (SEQ ID NO: 5; also
known as 2M+D614N polymerase).

Thus, in an aspect, there is provided a polymerase of an amino acid sequence having at least 36%, 50%, 60%, 70%, 80%, 90%, 95%, 99%, or 100% similarity or identity to the amino acid sequence of SEQ ID NO: 5, wherein residues 93, 141, 143, 409, 485, 521, 541, 545, 592, 614, and 664 are invariant (i.e. the mutations V93Q, D141A, E143A, Y409G, A485L, I521L, T541G, F545L, K592A, D614N, and E664K, are maintained).

In a particular embodiment, the nucleic acid polymerase may comprise or may be of the following amino acid sequence:

MILDTDYITEDGKPVIRIFKKENGEFKIDYDRNFEPYIYALLKDDSAIEDVKKITAERHGTTVRVV
RAEKVKKKFLGRPIEVWKLYFTHPQDQPAIRDKIKEHPAVVDIYEYDIPFAKRYLIDKGLIPMEGD
EELKMLAFAIATLYHEGEEFAEGPILMISYADEEGARVITWKNIDLPYVDVVSTEKEMIKRFLKVV
KEKDPDVLITYNGDNFDFAYLKKRSEKLGVKFILGREGSEPKIQRMGDRFAVEVKGRIHFDLYPVI
RRTINLPTYTLEAVYEAIFGQPKEKVYAEEIAQAWETGEGLERVARYSMEDAKVTYELGKEFFPME
AQLSRLVGQSLWDVSRSSTGNLVEWFLLRKAYERNELAPNKPDERELARRRESYAGGYVKEPERGL
WENIVYLDFRSIGPSIIITHNVSPDTLNREGCEEYDVAPQVGHKFCKDFPGFIPSLLGDLLEERQK
VKKKMKATIDPIEKKLLDYRQRLIKILANSFYGYYGYAKARWYCKECAESVTAWGRQYLETTIREI
EEKFGFKVLYADGDGFLATIPGADAETVKKKAKEFLDYINAKLPGLLELEYEGFYKRGFFVTKAKY
AVIDEEDKITTRGLEIVRRNWSEIAKETQARVLEAILKHGDVEEAVRIVKEVTEKLSKYEVPPEKL
VIYRQITRDLKDYKATGPHVAVAKRLAARGIKIRPGTVISYIVLKGSGRIGDRAIPFDEFDPAKHK
YDAEYYIENQVLPAVERILRAFGYRKEDLRYQKTRQVGLGAWLKPKT (SEQ ID NO: 6; also
known as 3M+D614N polymerase).

Thus, in an aspect, there is provided a polymerase of an amino acid sequence having at least 36%, 50%, 60%, 70%, 80%, 90%, 95%, 99%, or 100% similarity or identity to the amino acid sequence of SEQ ID NO: 6, wherein residues 93, 141, 143, 409, 485, 521, 541, 545, 592, 614, and 664 are invariant (i.e. the mutations V93Q, D141A, E143A, Y409G, A485L, I521L, T541G, F545L, K592A, D614N, and E664R, are maintained).

In some embodiments, the nucleic acid polymerase comprises the sequence:

(SEQ ID NO: 7
VLYXDGDGXLXXIPGAXXEXXXXXXXXXXXYINXKLXXXLELEYEGXYXRGFFXXKAKYAXXX

wherein X is any amino acid).

In other embodiments, the nucleic acid polymerase comprises the sequence:

(SEQ ID NO: 8
VLYXDGDGXLXXIPGAXXEXXXXXXXXXXYINXKLXXXLELEYEGXYXRGFFXXKGKYAXXX,

wherein X is any amino acid).

SEQ ID NO: 7 and SEQ ID NO: 8 are derived from a consensus sequence obtained after alignment of motifs C and KxY of polB-family polymerases (see FIG. 15 (Supp. FIG. 10)), where the “X” amino acids are not conserved and hence may tolerate a degree of variation. SEQ ID NO: 7 comprises the mutations T541G, F454L, and K592A. SEQ ID NO: 8 comprises the mutations T541G, F454L, and K592G.

Thus, in an aspect, there is provided a nucleic acid polymerase capable of producing a non-DNA nucleotide polymer from a nucleic acid template, said polymerase comprising amino acid sequence having at least 36%, 50%, 60%, 70%, 80%, 90%, 95%, 99%, or 100% similarity or identity to the amino acid sequence of SEQ ID NO: 1, and comprising SEQ ID NO: 7 or SEQ ID NO: 8. SEQ ID NO: 7 and SEQ ID NO: 8 are positioned from residue 536 of SEQ ID NO: 1 to residue 598 of SEQ ID NO: 1. The nucleic acid polymerase may also comprise any mutation or pattern of mutations disclosed herein. For instance, mutations V93Q, D141A, E143A, Y409G/Y409N, A485L, I521L/I521H, optionally D614N, and E664K/E664R. In a particular embodiment, the polymerase comprises the mutations V93Q, D141A, E143A, Y409G, A485L, I521L, optionally D614N, and E664K/E664R. The amino acid sequence of the polymerase may comprise SEQ ID NO: 7 or SEQ ID NO: 8 also including any mutations disclosed herein corresponding to positions D540, D542, K591, and/or K593 of SEQ ID NO: 1. These are positions 5, 7, 56, and 58 of SEQ ID NO: 7 and SEQ ID NO: 8.

In another aspect, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence comprises a E664R mutation.

The nucleic acid polymerase may comprise an amino acid sequence having at least 36%, 50%, 60%, 70%, 80%, 90%, 95%, 99%, or 100% similarity or identity to the amino acid sequence of SEQ ID NO: 1, wherein said amino acid sequence comprises a E664R mutation relative to SEQ ID NO: 1. The polymerase may include any other specific mutations or pattern of mutations as disclosed herein. For instance, the polymerase may also include: one or more, or all, of the following mutations: V93Q, D141A, E143A, and A485L relative to SEQ ID NO: 1: one or more, or all, of the following mutations: Y409, I521, and F545 relative to SEQ ID NO: 1; and/or one or more, or all, of the following mutations: Y409G, I521L or I521H, and F545L relative to SEQ ID NO: 1. The polymerase may include a D614 mutation relative to SEQ ID NO: 1, such as D614N.

In another aspect, there is provided a nucleic acid polymerase capable of producing a non-DNA nucleotide polymer from a nucleic acid template, said polymerase comprising amino acid sequence having at least 36%, 50%, 60%, 70%, 80%, 90%, 95%, 99%, or 100% similarity or identity to the amino acid sequence of SEQ ID NO: 1, wherein said amino acid sequence comprises mutations at any one of, all of, or any combination of positions D540, D542, K591, K593, Y663, and/or Q665 relative to SEQ ID NO: 1. In some examples, the mutation at any of positions D540, D542, K591, and/or K593 is to any less bulky residue, i.e. any residue that presents less of a steric block than the wild type residue. In some examples, the mutation at any of positions Y663, and/or Q665 is to any positively charged residue. In some embodiments, the mutation at D540 is D540A, D540G, D540S, or D540C. In particular, the mutation may be D540A. In some embodiments, the mutation at D542 is D542A, D542G, D542S, or D542C. In some embodiments, the mutation at K591 is K591G, K591A, K591C, K591M, K591S, K591D, K591P, K591N, K591T, K591E, K591V, K591Q, K591H, K591I, or K591L. In some embodiments, the mutation at K593 is K593G, K593A, K593C, K593M, K593S, K593D, K593P, K593N, K593T, K593E, K593V, K593Q, K593H, K593I, or K593L. In some embodiments, the E663 mutation may be E663K, E663R, or E663H. In some embodiments, the E665 mutation may be E665K, E665R, or E665H. The polymerase may include any other specific mutations or pattern of mutations as disclosed herein. In particular, any mutation at T541, K592, and/or E664 as disclosed herein. The polymerase may also include: one or more, or all, of the following mutations: V93Q, D141A, E143A, and A485L relative to SEQ ID NO: 1; one or more, or all, of the following mutations: Y409, I521, and F545 relative to SEQ ID NO: 1; and/or one or more, or all, of the following mutations: Y409G, I521L or I521H, and F545L relative to SEQ ID NO: 1. The polymerase may include a D614 mutation relative to SEQ ID NO: 1, such as D614N.

Polymerases of the present disclosure are capable of producing a non-DNA nucleotide polymer from a nucleic acid template. The nucleic acid template may be a DNA nucleotide polymer template. A non-DNA nucleotide means a nucleotide other than a deoxy ribonucleotide. The polymerases may be capable of incorporating 2′-O-methyl-RNA and (2′OMe) nucleotides and/or 2′-O-(2-methoxyethyl)-RNA (MOE) nucleotides into a polymer. The polymerases may also be capable of incorporating phosphorothioate 2′-O-2-methoxyethyl-RNA (PS-MOE) nucleotides and/or locked nucleic acid (LNA) nucleotides into a polymer.

The nucleic acid polymerase may be capable of acting upon a DNA primer to synthesise a 2′OMe, MOE, PS-MOE, or LNA polymer. The nucleic acid polymerase may be capable of acting upon a non-DNA primer to synthesise a 2′OMe, MOE, PS-MOE, or LNA polymer, for instance the polymerase may be capable of acting on a 2′OMe-RNA primer.

It will be appreciated that numerous polymerases of the present disclosure may show activity for multiple XNAs. As such, the polymerases may be capable of synthesising polymers or oligomers that comprises more than one type of XNA. For instance, polymers comprising both 2′OMe and MOE nucleotides.

To be considered capable of having the specified functions, the polymerase should be able to produce a polymer of at least 14 nucleotides in length, suitably at least 15 nucleotides in length; more suitably 40 nucleotides in length, most suitably at least 50 nucleotides in length. Thus, if polymerases of the disclosure are discussed as being capable of incorporating a particular type of XNA, it should be understood that the polymerase is expected to be able to consistently produce a polymer or at least 40 nucleotides, suitably at least 50 nucleotides in length.

Suitably, the polymers produced by the polymerases disclosed herein reflect the same four bases as conventional DNA polymers in terms of their information content, and correspond to the complementary bases of the template.

The polymerases disclosed herein, including the 2M polymerase, may be capable of acting upon the chemistries in the table below.

| Chemistry | NTPs |
|---|---|
| 2′-O-methyl-RNA | 2′OMe-NTPs |
| 2′-O-(2-methoxyethyl)-RNA | MOE-NTPs |
| Phosphorothioate 2′-O-(2- | PS-MOE-NTPs |
| methoxyethyl)-RNA | |
| LNA | INTPs |

The nucleic acid polymerase may be a polymerase which is capable of acting upon a DNA primer to synthesise an XNA molecule, such as a 2′OMe, MOE, PS-MOE, or LNA polymer, that is complementary to a single-stranded nucleic acid template. Such polymerases include polymerases comprising mutations corresponding to Y409G, I521L, T541G, F545L, K592A, and E664K (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1). The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations Y409G, I521L, T541G, F545L, K592A, and E664K relative to the amino acid sequence of SEQ ID NO: 1. In a particular embodiment, the nucleic acid polymerase which is capable of acting upon a DNA primer to synthesise a 2′OMe, MOE, PS-MOE, or LNA polymer, may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, Y409G, A485L, I521L, T541G, F545L, K592A, and E664K relative to the amino acid sequence of SEQ ID NO: 1.

### Polymerase

In principle, polymerases of the present disclosure may be made by introducing the specific mutations described herein into the corresponding site of a starting polymerase or ‘polymerase backbone’ of the operator's choice. In this way, the activity of that starting polymerase may be modified to provide the activities as described herein.

The polymerase backbone may be any member of the well-known polB enzyme family (including the pol delta variant which shows only 36% identity with the exemplary sequence of SEQ ID NO: 1). In some examples, the polymerase backbone may be any member of the well-known polB enzyme family excluding viral polymerases. The polymerase backbone may be any member of the well-known polB enzyme family having at least 36% identity to SEQ ID NO: 1; at least 50%; at least 60%; at least 70%; or at least 80%. At the 80% identity level, polB enzymes from the Archaeal Thermococcus and/or Pyrococcus genera are embraced. In a particular embodiment, the polymerase backbone has at least 90% identity to SEQ ID NO: 1.

Thus, in an example, there is provided a nucleic acid polymerase capable of producing a non-DNA nucleotide polymer from a nucleic acid template, said polymerase comprising amino acid sequence having at least 36% identity to the amino acid sequence of SEQ ID NO: 1, wherein said amino acid sequence is a polymerase from the polB family that includes any mutation or pattern of mutations disclosed herein relative to the amino acid sequence of SEQ ID NO: 1. In particular embodiments, the sequence is wild type apart from the specified mutations.

When using other polymerase backbones, mutations are transferred to the equivalent position as is well known in the art. For example, with reference to the exemplary polymerase 6G12, the following table illustrates how the transfer of mutations to alternate backbones may be carried out. The table shows Pol6G12 mutations and structural equivalent positions in other PolBs. The mutations found in Pol6G12 are shown against the underlying sequence of the wild-type Tgo. The structurally equivalent residue in other well-studied B-family polymerases is given. Residues that were not mapped to equivalent positions are shown as N.D.

| | | | |
|---|---|---|---|
| Tgo (1TGO) | Pol6G12 | RB69 (1IG9) | E. coli (3MAQ) |

| | | | | |
|---|---|---|---|---|
| V | 589 | A | 703 | 604 |
| E | 609 | K | 732 | N.D. |
| I | 610 | M | 733 | N.D. |
| K | 659 | Q | 778 | 681 |
| E | 664 | Q | 783 | 686 |
| Q | 665 | P | 784 | 687 |
| R | 668 | K | 788 | 690 |
| D | 669 | Q | 789 | 691 |
| K | 671 | H | N.D. | 693 |
| K | 674 | R | 792 | N.D. |
| T | 676 | R | 801 | 700 |
| A | 681 | S | 806 | 705 |
| L | 704 | P | 835 | 733 |
| E | 730 | G | 869 | 750 |

The polymerase may be a fragment of a polymerase which retains the polymerase function.

### Reference Sequence

When particular amino acid residues of polymerase are referred to using numeric addresses, the numbering is taken with reference to the true wild type amino acid sequence of SEQ ID NO: 1 (or to the nucleic acid sequence encoding same).

This is to be used as is well understood in the art to locate the residue of interest. This is not always a strict counting exercise—attention must be paid to the context. For example, if the protein of interest is of a slightly different length, then location of the correct residue in that sequence corresponding to (for example) E664 may require the sequences to be aligned and the equivalent or corresponding residue picked, rather than simply taking the 664th residue of the sequence of interest. This is well within the ambit of the skilled reader.

“Mutation” may refer to the substitution or truncation or deletion of the residue, motif or domain referred to. In a particular embodiment, the mutation is a substitution of one type of amino acid residue for another type of amino acid residue.

Mutation may be effected at the polypeptide level e.g. by synthesis of a polypeptide having the mutated sequence, or may be effected at the nucleotide level e.g. by making a nucleic acid encoding the mutated sequence, which nucleic acid may be subsequently translated to produce the mutated polypeptide. Where no amino acid is specified as the replacement amino acid for a given mutation site, as a default alanine (A) may be used. Suitably the mutations used at particular site(s) are as set out herein.

A fragment is suitably at least 10 amino acids in length, suitably at least 25 amino acids, suitably at least 50 amino acids, suitably at least 100 amino acids, or suitably the majority of the polymerase polypeptide of interest i.e. 387 amino acids or more, suitably at least 50 amino acids, suitably at least 600 amino acids, suitably at least 700 amino acids, suitably the entire 773 amino acids of the Tgo or TgoT polB sequence.

### Sequence Variation

The polymerases of the present disclosure may comprise sequence changes relative to the wild type sequence in addition to the key mutations described in more detail herein. Specifically the polymerases of the present disclosure may comprise sequence changes at sites which do not significantly compromise the function or operation of the polymerase as described herein.

Polymerase function may be easily tested by operating the polymerase as described, such as in the examples section, in order to verify that function has not been abrogated or significantly altered.

Thus, provided that the polymerase retains its function which can be easily tested as set out herein, sequence variations may be made in the polymerase molecule relative to the wild type reference sequence.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In considering what mutations, substitutions or other such changes might be made relative to the wild type sequence, retention of the function of the polymerase is paramount. Typically conservative amino acid substitutions would be less likely to adversely affect the function. Suitably the polymerase of the present disclosure varies from the wild type sequence only by conservative amino acid substitutions except as discussed.

### Sequence Similarity/Identity

Sequence comparisons can be conducted with the aid of readily available sequence comparison programs. These publicly and commercially available computer programs can calculate sequence identity between two or more sequences.

The skilled technician will appreciate how to calculate the percentage identity between two nucleic sequences. In order to calculate the percentage identity between two nucleic sequences, an alignment of the two sequences must first be prepared, followed by calculation of the sequence identity value. The percentage identity for two sequences may take different values depending on: (i) the method used to align the sequences, for example, the Needleman-Wunsch algorithm (e.g. as applied by Needle (EMBOSS) or Stretcher (EMBOSS), the Smith-Waterman algorithm (e.g. as applied by Water (EMBOSS)), or the LALIGN application (e.g. as applied by Matcher (EMBOSS); and (ii) the parameters used by the alignment method, for example, local versus global alignment, the matrix used, and the parameters applied to gaps.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length-dependent. Therefore, the shorter a pair of sequences is, the higher the sequence identity one may expect to occur by chance.

A calculation of percentage identities between two nucleic acid sequences may then be calculated from such an alignment as (N/T)*100, where N is the number of positions at which the sequences share an identical residue, and T is the total number of positions compared including gaps but excluding overhangs.

The sequence alignment may be a pairwise sequence alignment. Suitable services include Needle (EMBOSS), Stretcher (EMBOSS), Water (EMBOSS), Matcher (EMBOSS), LALIGN, or GeneWise. In an example, the identity between two amino acid sequences may be calculated using the service Needle (EMBOSS) set to the default parameters, e.g. matrix (BLOSUM62), gap open (10), gap extend (0.5), end gap penalty (false), end gap open (10), and end gap extend (0.5). In another example, the identity between two amino acid sequences may be calculated using the service Matcher (EMBOSS) set to the default parameters, e.g. matrix (BLOSUM62), gap open (14), gap extend (4), alternative matches (1). In an example, the identity between two nucleic acid sequences may be calculated using the service Needle (EMBOSS) set to the default parameters, e.g. matrix (DNA full), gap open (10), gap extend (0.5), end gap penalty (false), end gap open (10), and end gap extend (0.5). In another example, the identity between two nucleic acid sequences may be calculated using the service Matcher (EMBOSS) set to the default parameters. e.g. matrix (DNAfull), gap open (16), gap extend (4), alternative matches (1).

Suitably identity or similarity is assessed at the amino acid level over at least 400 or 500, preferably 600, 700, or 773 amino acids with the relevant polypeptide sequence(s) disclosed herein (such as any one of SEQ ID NOs: 1 to 6).

Similarity or identity may be calculated by comparing the full-length of an amino acid sequence of a truncated nucleic acid polymerase to the relevant portion of a reference sequence (such as any one of SEQ ID NOs: 1 to 6). In particular embodiments, the similarity or identity is calculated taking into account the full-length of the reference sequence (e.g. all 773 residues of any one of SEQ ID NOs: 1 to 6). In a certain embodiment, the sequence identity of a nucleic acid of the present disclosure is calculated as the percentage of identity to the full 773 residues of any one of SEQ ID NOs: 1 to 6.

Suitably, similarity or identity should be considered with respect to one or more of those regions of the sequence known to be essential for protein function rather than non-essential neighbouring sequences. This is especially important when considering homologous sequences from distantly related organisms.

When considering conserved regions, suitably the 36% of residues common to both SEQ ID NO: 1 and to the pol delta member of the polB enzyme family should be taken to be potentially important residues which are suitably not mutated in the polypeptide of the present disclosure unless otherwise discussed. Thus suitably the polypeptide of the present

disclosure has at least 36% identity to SEQ ID NO: 1 and suitably the amino acid residues making up said at least 36% identity comprise the amino acid residues corresponding to those which are identical between SEQ IN NO: 1 and the pol delta member of the polB enzyme family. Suitably the polypeptide of the present disclosure has at least 36% identity to SEQ ID NO: 1 and has at least 36% identity to the pol delta member of the polB enzyme family.

For comparison purposes, the sequence of the human DNA polymerase delta catalytic subunit is provided in the following sequence:

(SEQ ID NO: 9)
MDGKRRPGPGPGVPPKRARGGLWDDDDAPRPSQFEEDLALMEEMEAEHRLQEQEEEELQSVLEGVA
DGQVPPSAIDPRWLRPTPPALDPQTEPLIFQQLEIDHYVGPAQPVPGGPPPSRGSVPVLRAFGVTD
EGFSVCCHIHGFAPYFYTPAPPGFGPEHMGDLQRELNLAISRDSRGGRELTGPAVLAVELCSRESM
FGYHGHGPSPFLRITVALPRLVAPARRLLEQGIRVAGLGTPSFAPYEANVDFEIRFMVDTDIVGCN
WLELPAGKYALRLKEKATQCQLEADVLWSDVVSHPPEGPWQRIAPLRVLSFDIECAGRKGIFPEPE
RDPVIQICSLGLRWGEPEPFLRLALTLRPCAPILGAKVQSYEKEEDLLQAWSTFIRIMDPDVITGY
NIQNFDLPYLISRAQTLKVQTFPFLGRVAGLCSNIRDSSFQSKQTGRRDTKVVSMVGRVQMDMLQV
LLREYKLRSYTLNAVSFHFLGEQKEDVQHSIITDLQNGNDQTRRRLAVYCLKDAYLPLRLLERLMV
LVNAVEMARVTGVPLSYLLSRGQQVKVVSQLLRQAMHEGLLMPVVKSEGGEDYTGATVIEPLKGYY
DVPIATLDFSSLYPSIMMAHNICYTILLRPGTAQKLGLTEDQFIRTPTGDEFVKTSVRKGLLPQIL
ENLLSARKRAKAELAKETDPLRRQVLDGRQLALKVSANSVYGFTGAQVGKLPCLEISQSVTGFGRQ
MIEKTKQLVESKYTVENGYSTSAKVVYGDTDSVMCRFGVSSVAEAMALGREAADWVSGHFPSPIRL
EFEKVYFPYLLISKKRYAGLLFSSRPDAHDRMDCKGLEAVRRDNCPLVANLVTASLRRLLIDRDPE
GAVAHAQDVISDLLCNRIDISQLVITKELTRAASDYAGKQAHVELAERMRKRDPGSAPSLGDRVPY
VIISAAKGVAAYMKSEDPLFVLEHSLPIDTQYYLEQQLAKPLLRIFEPILGEGRAEAVLLRGDHTR
CKTVLTGKVGGLLAFAKRRNCCIGCRTVLSHQGAVCEFCQPRESELYQKEVSHLNALEERFSRLWT
QCQRCQGSLHEDVICTSRDCPIFYMRKKVRKDLEDQEQLLRRFGPPGPEAW

Thus, the polymerase may comprise an amino acid sequence having at least 36%, 50%, 60%, 70%, 80%, 90%, 95%, 99%, or 100% similarity or identity to the amino acid sequence of SEQ ID NO: 1 and at least 36%, 50%, 60%, 70%, 80%, 90%, 95%, 99%, or 100% similarity or identity to the amino acid sequence of SEQ ID NO: 9. The polymerase may comprise an amino acid sequence having at least 36%, 50%, 60%, 70%, 80%, 90%, 95%, 99%, or 100% identity to the amino acid sequence of SEQ ID NO: 1 and at least 36%, 50%, 60%, 70%, 80%, 90%, 95%, 99%, or 100% identity to the amino acid sequence of SEQ ID NO: 9.

The same considerations apply to nucleic acid nucleotide sequences.

### Truncations

Truncations of the overall full-length polymerase enzyme of the present disclosure may be made if desired. Suitably full-length polymerase polypeptide is used as the backbone polypeptide, such as full length Tgo polymerase 1-773 as shown in any one of SEQ ID NOs: 1 to 6. Any truncations used should be carefully checked for activity. This may be easily done by assaying the enzyme(s) as described herein.

### Purification

Polymerases of the present disclosure are advantageously thermo-stable. By expressing these polymerases in a conventional (non thermo-stable) host strain, purification is advantageously simplified. For example, when the polymerases of the present disclosure are expressed in a conventional non thermo-stable host cell, approximately 90% purity may be obtained simply by heating the host cells to 99° C. followed by centrifugal removal of cellular debris. Higher purity levels may easily be obtained for example by subjecting the heat treated soluble fraction of the host cell to ion exchange and/or heparin column purifications.

Suitably the polymerase of the present disclosure is not fused to any other polypeptide. Suitably the polymerase of the present disclosure is not tagged with any further polypeptides or fusions.

### Fidelity

It is clearly important that sufficient fidelity is maintained for accurate production (or reproduction) of the nucleic acid polymers. Suitably polymerases of the present disclosure retain at least 95% fidelity. Fidelity (error threshold) may be taken as the number of errors introduced divided by the number of nucleotides polymerised. In other words, an error rate of 1% equates to the introduction of one error for every 100 nucleotides polymerised. In fact, the polymerases of the present disclosure attain a much better fidelity than this. An error rate of 5% or less is considered as the minimum useful fidelity level for the polymerases of the present disclosure; suitably the polymerases of the present disclosure have an error rate of 4% or less; suitably 3% or less; suitably 2% or less; suitably 1% or less.

Fidelity may be assessed as aggregate fidelity (e.g. DNA-XNA-DNA) which thus encompasses two conversion events (DNA-XNA and XNA-DNA); the figures should be adjusted or interpreted accordingly.

### Methods and Uses

The polymerases disclosed herein may be used to generate XNA polymers. Thus, in an aspect, there is provided a method for making a non-DNA nucleotide polymer, said method comprising contacting a nucleic acid template with any nucleic acid polymerase disclosed herein, under conditions conducive to polymerisation.

The non-DNA nucleotide polymer may comprise or consist of 2′OMe-RNA nucleotides and/or MOE-RNA nucleotides. As such, 2′OMe-RNA nucleotides and/or MOE-RNA nucleotides may be provided during the polymerisation. In an embodiment, the resultant polymer is an all 2′OMe-RNA polymer. In another embodiment, the resultant polymer is an all MOE-RNA polymer. In an additional embodiment, the resultant polymer comprises both 2′OMe-RNA and MOE-RNA. The polymer may include only 2′OMe-RNA and MOE-RNA. The polymer may be an oligonucleotide.

The non-DNA nucleotide polymer may comprise phosphorothioate 2′-O-2-methoxyethyl-RNA (PS-MOE) nucleotides or locked nucleic acid (LNA) nucleotides. As such, PS-MOE nucleotides and/or LNA nucleotides may be provided during the polymerisation.

In an embodiment, the method comprises the provision of 2′OMe-RNA nucleotides, MOE-RNA nucleotides, PS-MOE nucleotides, LNA nucleotides, or any combination of said nucleotides to the polymerisation reaction.

The method may comprise the provision of a primer, for instance a DNA or non-DNA primer. The primer may be a 2′OMe-RNA primer.

The method may be used to generate a polymer of at least 14, 15, 20, 25, 40, 50, or 70 nucleotides in length.

In another aspect, there is provided the use of any nucleic acid polymerase disclosed herein for the generation of a non-DNA nucleotide polymer. The use may be for the generation of an oligonucleotide. The polymer may comprise 2′OMe-RNA nucleotides, MOE-RNA nucleotides, PS-MOE nucleotides, LNA nucleotides, or any combination. The polymer may comprise 2′OMe-RNA nucleotides. The polymer may comprise MOE-RNA nucleotides. The polymer may comprise 2′OMe-RNA nucleotides and MOE-RNA nucleotides. The polymer may be an all 2′OMe-RNA polymer. The polymer may be an all MOE-RNA polymer. The polymer may include only 2′OMe-RNA and MOE-RNA.

In some examples, the resultant polymers are capable of acting as catalysts. The polymers may be endonucleases. The catalytic polymers may comprise 2′OMe-RNA and/or MOE-RNA. The catalytic polymers may include only 2′OMe-RNA nucleotides. The polymers may include only 2′OMe-RNA nucleotides and have endonuclease activity (2′OMezymes).

In some examples, the resultant polymers are aptamers. The aptamers may comprise 2′OMe-RNA and/or MOE-RNA. The aptamers may include only 2′OMe-RNA, only MOE-RNA, or only 2′OMe-RNA and MOE-RNA.

In another aspect, there is provided the use of a nucleic acid polymerase disclosed herein to extend a DNA primer immobilised on a substrate to synthesise a non-DNA nucleic acid molecule that is complementary to a single-stranded nucleic acid template.

### Products

In an aspect, there is provided a catalytic oligonucleotide, wherein the nucleotides include only 2′OMe-RNA nucleotides. The catalytic oligonucleotide may have endonuclease activity. The oligonucleotide may have the sequence of a 2′OMezyme disclosed herein.

In another aspect, there is provided any aptamer as disclosed herein.

### Remarks

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### EXAMPLES

Steric exclusion is a key element of enzyme substrate specificity, including in polymerases. Here the inventors describe the discovery of a two-residue, nascent strand, steric control “gate” in an archaeal DNA polymerase. It is shown that engineering of the gate to reduce steric bulk in the context of a previously-described RNA polymerase activity unlocks the synthesis of 2′-modified RNA oligomers, specifically the efficient synthesis of both defined and random-sequence 2′-O-methyl-RNA (2′OMe-RNA) and 2′-O-(2-methoxyethyl)-RNA (MOE-RNA) oligomers up to 750 nt.

This enabled the discovery of RNA endonuclease catalysts entirely composed of 2′OMe-RNA (“2′OMezymes”) for the allele-specific cleavage of oncogenic KRAS (G12D) and β-catenin CTNNB1 (S33Y) mRNAs, and the elaboration of mixed 2′OMe-/MOE-RNA aptamers with high affinity for Vascular Endothelial Growth Factor (VEGF). Our results open up these chemistries—used in several approved nucleic acid therapeutics—for enzymatic synthesis and a wider exploration in directed evolution and nanotechnology.

### Example 1—A Two-Residue Nascent Strand Steric Gate Controls 1 Synthesis of 2′-O-methyl and 2′-O-(2-methoxyethyl)-RNA

In the experiments discussed below, the inventors disclose the existence of a two-residue steric gate in Tgo, the replicative DNA polymerase from the hyperthermophilic archaeon Thermococcus gorgonarius. Mutation of this steric gate in the context of an earlier engineered primer-dependent RNA polymerase activity in Tgo^{10, 11} enabled exceptionally efficient synthesis of 2′OMe-RNA and, for the first time, MOE-RNA. This also allowed in vitro evolution of the first all-2′OMe-RNA catalysts (“2′OMezymes”) for mutation-specific cleavage of two oncogenic mRNA targets as well as the elaboration of mixed 2′OMe/MOE-RNA aptamers with high affinity for Vascular Endothelial Growth Factor (VEGF).

### Results

We had previously observed that engineered versions of Tgo, specifically TGK and TGLLK (Tgo: V93Q, D141A, E143A, Y409G, A485L, I521L, F545L, E664K)^{10, 11} (FIG. 1b) had a capacity for RNA, 2′F-DNA and to a lesser extent 2′OMe-RNA synthesis. However, 2′OMe-RNA synthesis by TGLLK was comparatively inefficient, especially on the more challenging N₄₀ random-sequence templates often used in in vitro selection experiments. We sought to improve 2′OMe-RNA synthesis by quasi-rational design based on systematic elimination of unfavourable steric contacts between the bulky 2-methoxy substituents of the 2′OMe-RNA nascent strand and the polymerase, using a simple static model of 2′OMe-RNA synthesis comprising the ternary structure of the homologous DNA polymerase from T. kodakarensis KOD1 (PDB ID 5OMF)¹², and the structure of an RNA-DNA duplex¹³ augmented with 2′-O-methyl groups adjusted to C1′-C2′-O2′-C_{Methyl} dihedral angles of 71°^{14, 15} (gauche conformation).

This approach identified the sidechains of Tgo residues D540, T541, K592, D614, and E664 as proximal and potentially sterically clashing with 2-methoxy groups in the 2′OMe-RNA nascent strand. These residues were targeted for site-saturation mutagenesis in the TGLLK framework and screened for 2′OMe-RNA synthesis activity (SI FIG. 1). Among these, T541 was of particular interest as it makes direct contact with the 3′-end nucleotide of the nascent (primer) strand, the positioning of which is crucial for catalysis, i.e., the nucleophilic attack of the nascent strand terminal 3′-OH on the a-phosphate of the incoming nucleoside triphosphate substrate. Indeed, the screen identified T541G as a mutation that increased 2′OMe-RNA synthesis activity, as well as mutations K592A and K664R, which led to slight increases in activity. Combining mutations revealed striking synergy of the T541G and K592A mutations for 2′OMe-RNA synthesis in the context of the previous TGLLK mutations (SI FIG. 1, FIG. 1e).

Polymerase TGLLK: T541G, K592A (henceforth named 2M) (FIG. 1) showed a striking increase in 2′OMe-RNA synthesis activity on a model DNA template containing all possible dinucleotide combinations (TempN)¹⁶ (FIG. 1f) as well as on a random sequence N₄₀ template (FIG. 1g). Furthermore, 2M enabled long-range 750 nt 2′OMe-RNA synthesis (FIG. 1h). This suggests that residues T541 and K592 together pose a strong block to 2′OMe-RNA synthesis, which is relieved by mutation to less bulky side-chains (T541G, K592A) (FIG. 1d). The 2M mutations also appear to reshape the polymerase primer-binding interface to the extent that both DNA and to an even greater extent 2′OMe-RNA synthesis are disfavoured from a DNA primer compared to a 2′OMe-RNA primer (SI FIG. 1). Nevertheless, these mutations do not seem to impede nucleobase discrimination as fidelity measurements suggest that the error rate of 2M synthesizing 2′OMe-RNA is in the same range as its parent polymerases TGLLK and TGK synthesizing 2′OMe-RNA and RNA, respectively (SI Table 4).

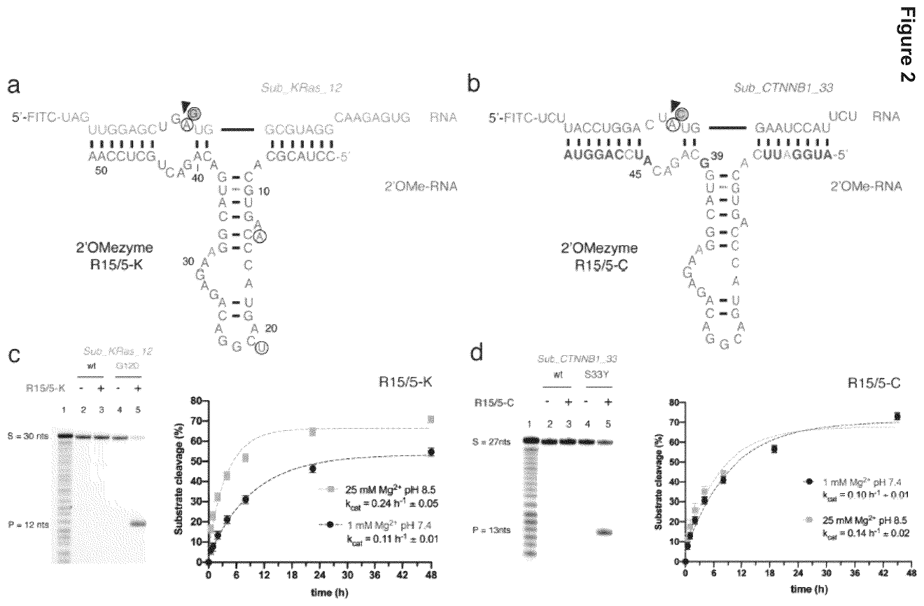

Poor efficiency of XNA synthesis and reverse transcription from random templates can cause synthetic biases and undersampling of the sequence space with concomitant loss of library diversity, which leads to suboptimal outcomes in repertoire selection experiments. We reasoned that the enhanced efficiency of 2′OMe-RNA synthesis by 2M (together with the recently described more efficient 2′OMe-RNA reverse transcriptase C8¹⁷) might allow success in previously intractable in vitro evolution experiments. To this end, we pursued de novo selection of fully-2OMe-RNA catalysts (henceforth called 2′OMezymes), which to our knowledge had not previously been described. Starting directly from random-sequence fully-2′OMe-RNA (N₄₀) repertoires with RNA substrates covalently attached for cleavage in cis¹⁸, we sought to discover endonuclease 2′OMezymes targeted to the KRAS oncogene mRNA. After 15 rounds, the selection pool was deep sequenced, screened for RNA endonuclease activity, and the most abundant active sequence subjected to another five rounds of catalytic ‘maturation’ selection from a doped sequence library (70% correct base, 10% each of the alternative bases). The most enriched 2′OMezyme sequence R15/5-KRAS (henceforth called R15/5-K) (FIG. 2a) was prepared by solid-phase synthesis for further characterization.

R15/5-K is a highly sequence-specific RNA endonuclease that catalyzes cleavage of its cognate substrate, the KRAS G12D (cG35A) RNA, in a bimolecular reaction (k_{cat}=0.24 h⁻¹±0.05 in 25 mM Mg²⁺, pH 8.5, 37° C.) (FIG. 2c), and is capable of multiple-turnover catalysis (SI FIG. 2).

Cleavage is G12D (c.35G>A) mutation-specific with essentially no cleavage of ‘wild type’ (wt) KRAS RNA, which differs by only one nucleotide (G35) (FIG. 2c). Furthermore, unlike comparable variants of the canonical 10-23 DNAzyme targeting the same KRAS sequence motif, R15/5-K was able to invade and cleave not just short model RNA substrates, but a long, structured 2.1 kb KRAS transcript, retaining its specificity for the G12D mutation (c.35G>A) (FIG. 2e), with virtually no cleavage of the wt KRAS transcript or a transcript with a similar nearby oncogenic mutation (G13D (c.38G>A)).

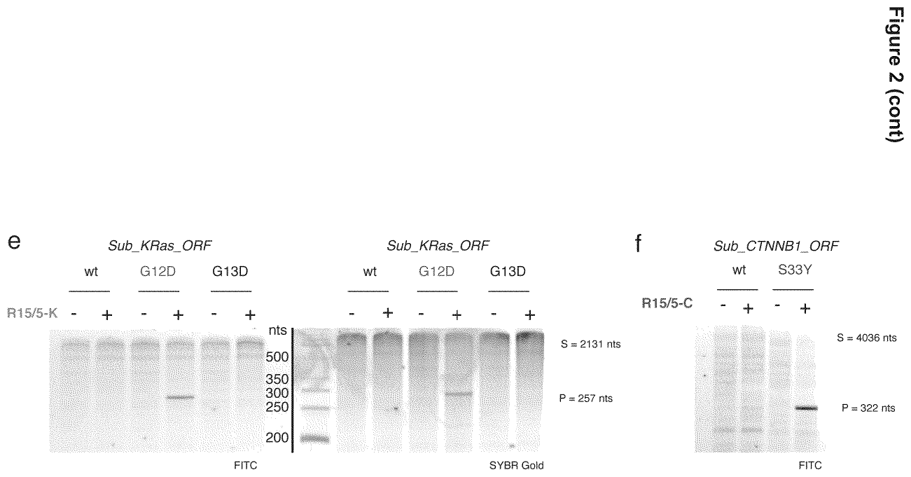

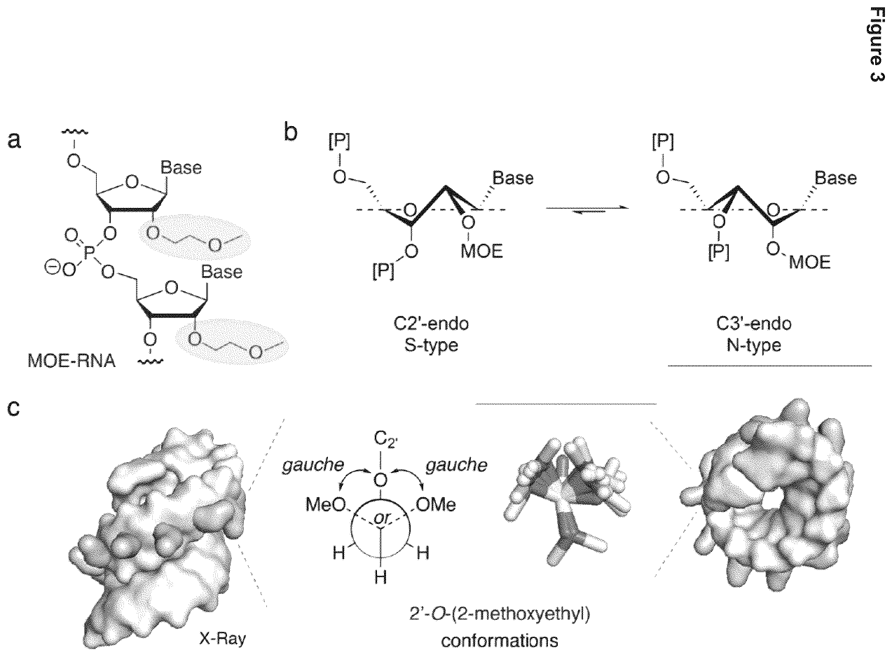

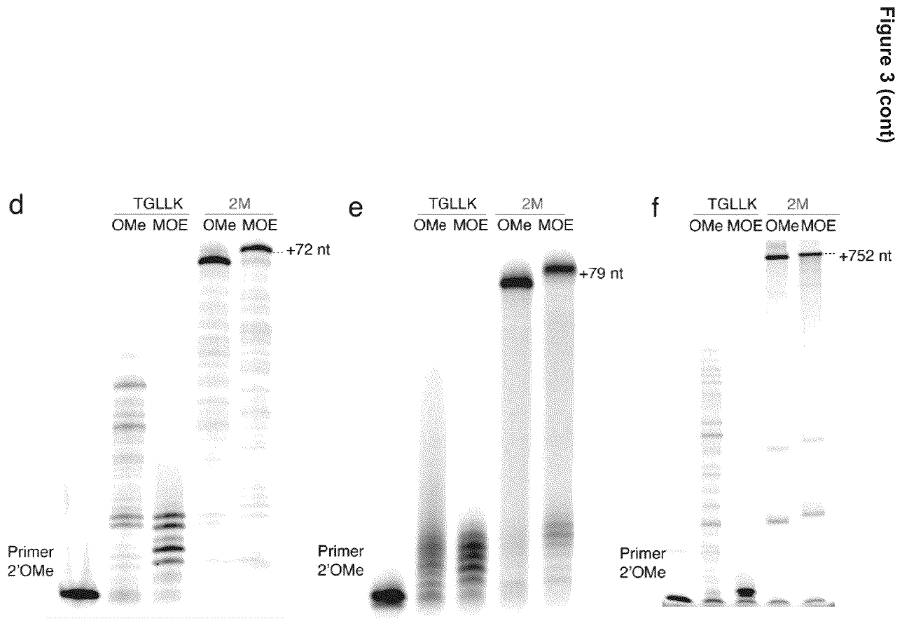

As observed previously in RNA endonuclease DNA- and XNAzymes (and some ribozymes), cleavage proceeds through transesterification and a 2′,3′-cyclic phosphate (>p) intermediate as shown by MALDI-ToF mass spectrometry and electrophoretic mobility shift (EMSA) analysis of cleavage products (SI FIG. 3). However, while RNA endonuclease DNA- and XNAzymes are obligatory metalloenzymes, dependent on the presence of divalent cations (typically Mg²⁺) for both folding and catalysis, and therefore exhibit a substantial loss in catalytic activity under physiological conditions, the R15/5-K 2′OMezyme retained 70-80% activity under a quasi-physiological low-Mg²⁺ regime (0.5-1 mM Mg²⁺) (FIG. 2c) over a broad pH range (SI FIG. 2). Indeed, even the single-turnover rate was only reduced by approximately 50% compared with optimal conditions (k_{cat}=0.11 h⁻¹±0.01 in 1 mM Mg²⁺, pH 7.4, 37° C.) (FIG. 2c). Furthermore, unlike the 10-23 DNAzyme, RNA cleavage activity of R15/5-K could even be observed in the absence of Mg²⁺, albeit at a very low rate (k_{cat}=0.001 h⁻¹±0.0002 in 5 mM EDTA, pH 7.4, 37° C.) (SI FIG. 2). Finally, as expected due to its all-2′OMe-RNA makeup, R15/5-K proved highly biostable with no significant degradation (or loss in activity) after incubation in human serum at 37° C. for 120 h (SI FIG. 4).

The potential for modularity, i.e. programmability of RNA target specificity through their binding arms, is an attractive feature of some nucleic acid catalysts like the 10-23 DNAzyme, but is not shared by all. We next explored whether the R15/5-K 2′OMezyme could be retargeted to an alternative mRNA substrate. Based on the putative secondary structure of R15/5-K (FIG. 2a) we reprogrammed nucleotides 1-7, 39-40, 45-51, flanking the central hairpin motif, to pair to the β-catenin (CTNNB1) proto-oncogene mRNA (c.85-111). The resulting 2′OMezyme R15/5-CTNNB1 was only weakly active, but an improved variant (R15/5-CTNNB1: A39G, U45A, hence forth called R15/5-C) (FIG. 2b) was readily discovered by screening mutations of residues flanking the recognition elements (position 9, 39, 42 & 45) (SI FIG. 5). The improved 2′OMezyme R15/5-C was highly specific and only able to cleave the oncogenic S33Y CTNNB1 (c.G99A) RNA substrate (FIG. 2d). It retained the capability for multi-turnover catalysis (SI FIG. 2) and invasion of long (4 kb), structured complete β-catenin transcript, while retaining its specificity (FIG. 2f). Although the R15/5-C turnover rate was ˜40% lower compared to the parent R15/5-K under optimal conditions (k_{cat}=0.14 h⁻¹±0.02 in 25 mM Mg²⁺, pH 8.5, 37° C.), re-targeting did not affect the rate under quasi-physiological low-Mg²⁺ conditions (k_{cat}=0.10 h⁻¹±0.01 in 1 mM Mg²⁺, pH 7.4, 37° C.) (FIG. 2d).

Next we wondered if the 2M polymerase would also be able to cope with more challenging 2′-modified RNA substrates. Among these, the 2′-O-(2-methoxyethyl) (MOE) modification (FIG. 3a) is of special interest because of the superior biophysical and pharmacological properties of the MOE-modified nucleic acid. In both 2′OMe- and MOE-RNA, the 2′-substituents favour a C3′-endo sugar conformation of the ribofuranose ring (akin to the ribose sugar puckering in RNA (A-form)) (FIG. 3b). The MOE ethylene glycol monomethyl ether modification is favoured in an extra gauche orientation along O_{2′}—C—C—O (FIG. 3c), extending the gauche effect from O_{4′}—C₁—C_{2′}—O_{2′} and thereby driving the rotational equilibrium to C3′-endo (FIG. 3b)¹⁹. This structural pre-organization (and rigidity of the MOE-RNA structure) enhances base-pairing and stacking interactions with target RNA and leads to a high antisense binding affinity of 2′OMe- and MOE-RNA to RNA. Indeed, every single MOE modification in a DNA oligo increases the Tₘ of the oligo bound to its complementary RNA by 0.9-1.2° C.¹⁹.

In addition, the gauche-oriented MOE moiety places an additional hydrogen bond acceptor in the minor groove, which favours the formation of a hydrogen bonding network. Thereby, the MOE modifications lead to stabilization of up to three water molecules trapped between the MOE moiety and the phosphodiester backbone²⁰. This hydration “spine” together with steric hindrance introduced by the 2′-O-(2-meth-oxyethyl) group in the minor groove leads to shielding of the 5′-3′ phosphodiester linkage, resulting in exceptional biostability and in vivo half-life of MOE-RNA¹, and the excessive hydration increases paracellular absorption and intestinal uptake rate of MOE-modified oligonucleotides compared to unmodified oligos²¹.

However, solution-state NMR²² and X-ray crystallography²⁰ structures indicate a challenging steric envelope of the MOE-RNA helix for enzymatic synthesis with the bulky methoxyethyl groups, adopting the aforementioned gauche conformation and projecting away from the helical envelope (FIG. 3c). Nevertheless, we undertook chemical synthesis of MOE-NTPs to explore enzymatic MOE-RNA synthesis.

Synthesis of the MOE-nucleosides²³ and their phosphoramidites²⁴ is established and commercial synthesis of MOE-oligonucleotides is available, but the 2′-O-(2-methoxyethyl)nucleoside triphosphates (MOE-NTPs) were neither commercially available nor was their synthesis established. We therefore first developed a synthetic route to the four MOE-NTPs starting from the commercially available 2′-O-(2-methoxyethyl)ribonucleosides by triphosphorylation based on the established Ludwig method^{25, 26} (SI FIG. 6, SI Materials & Methods).

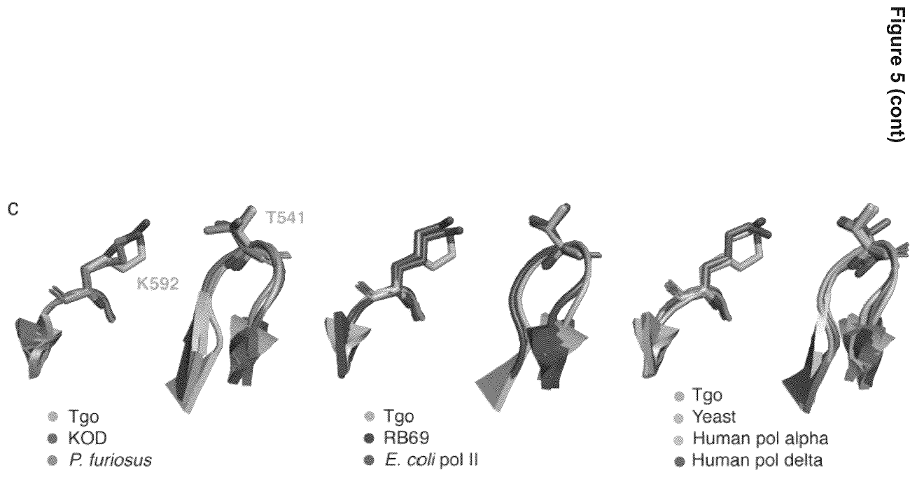

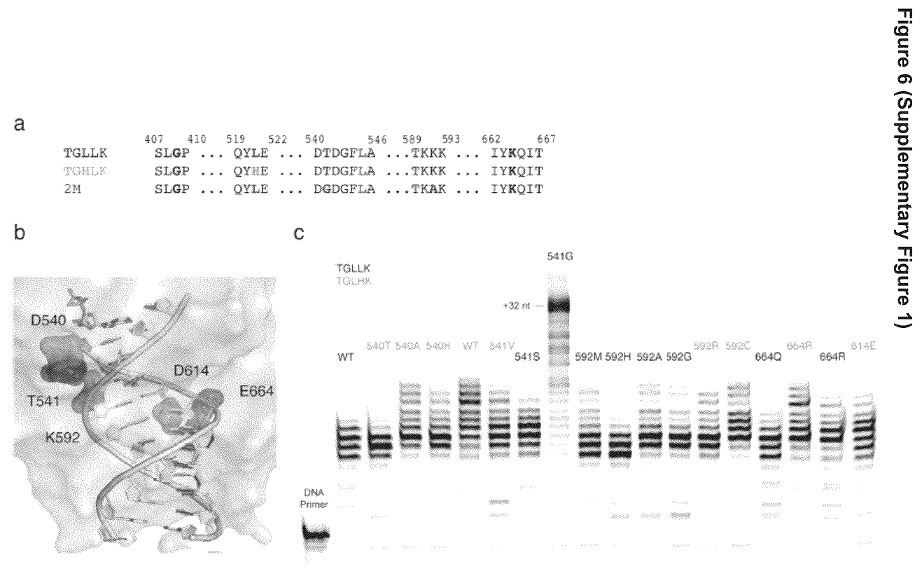

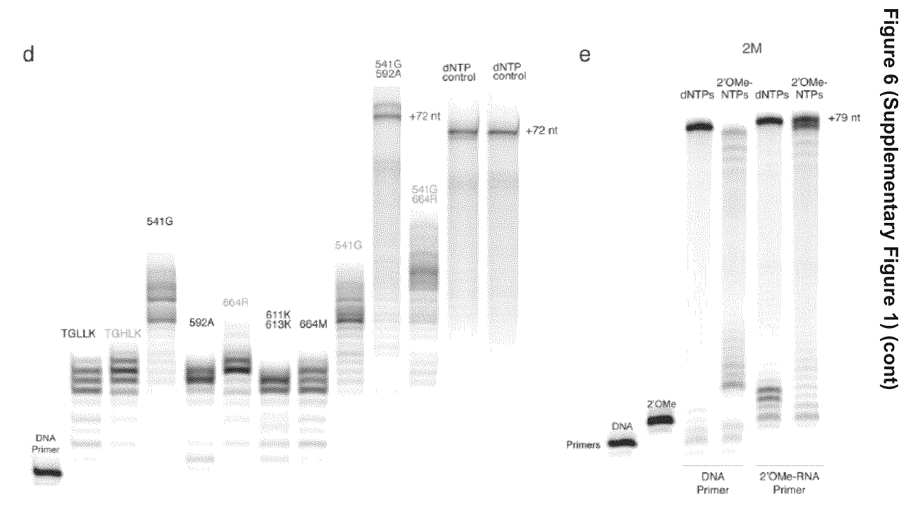

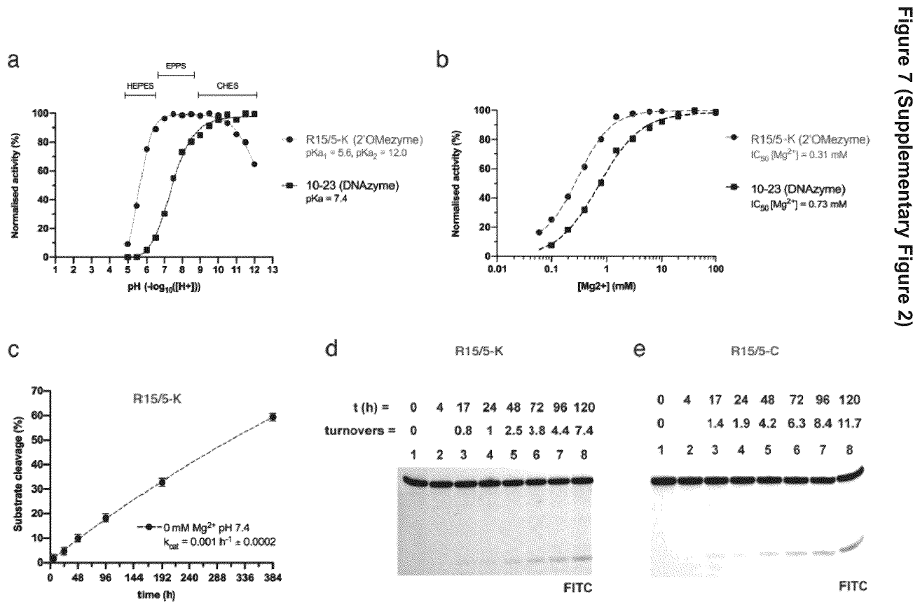

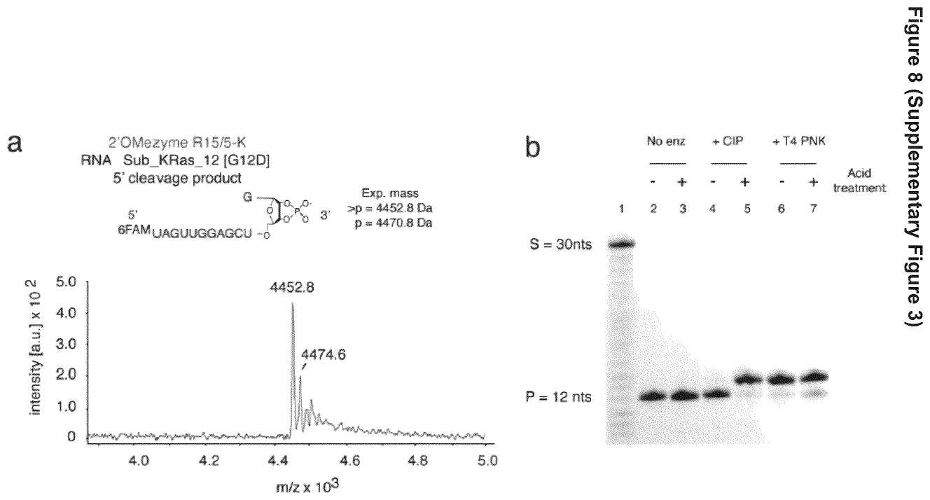

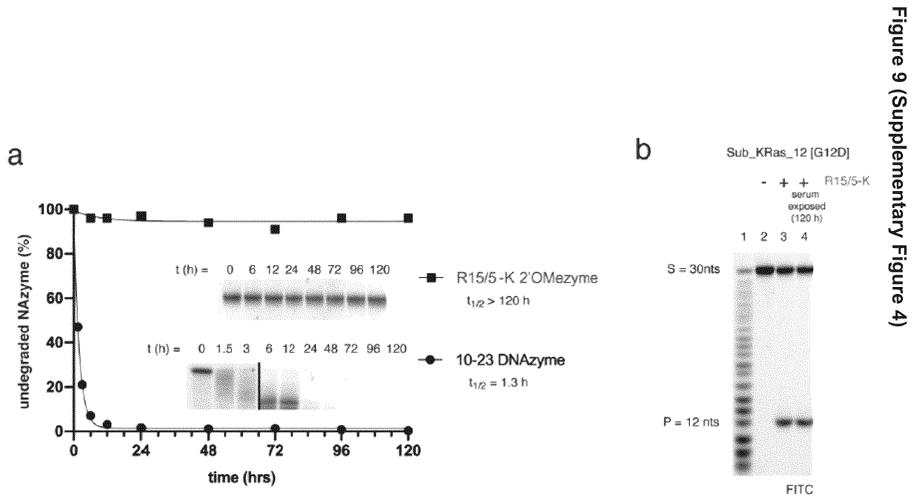

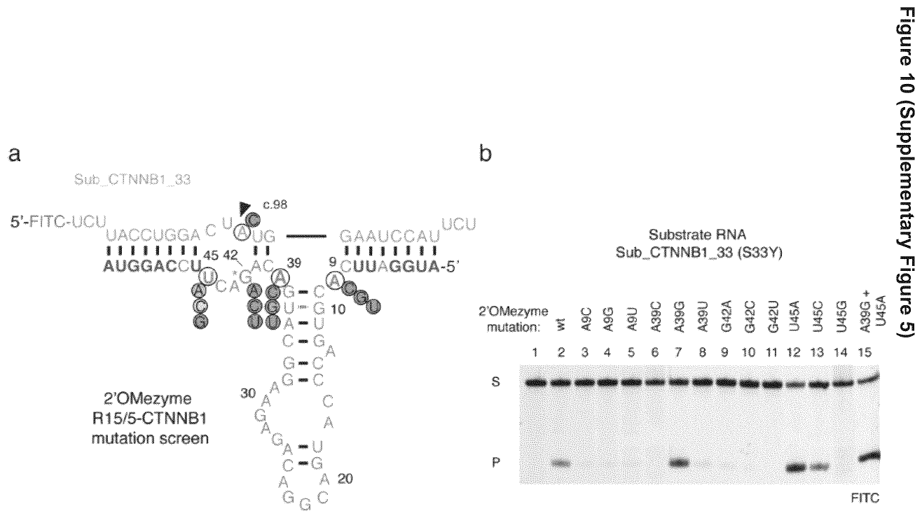

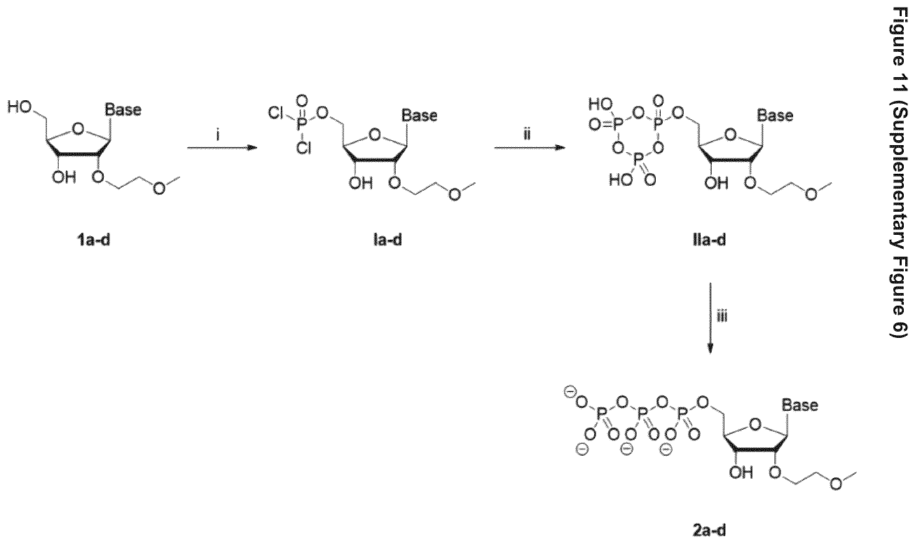

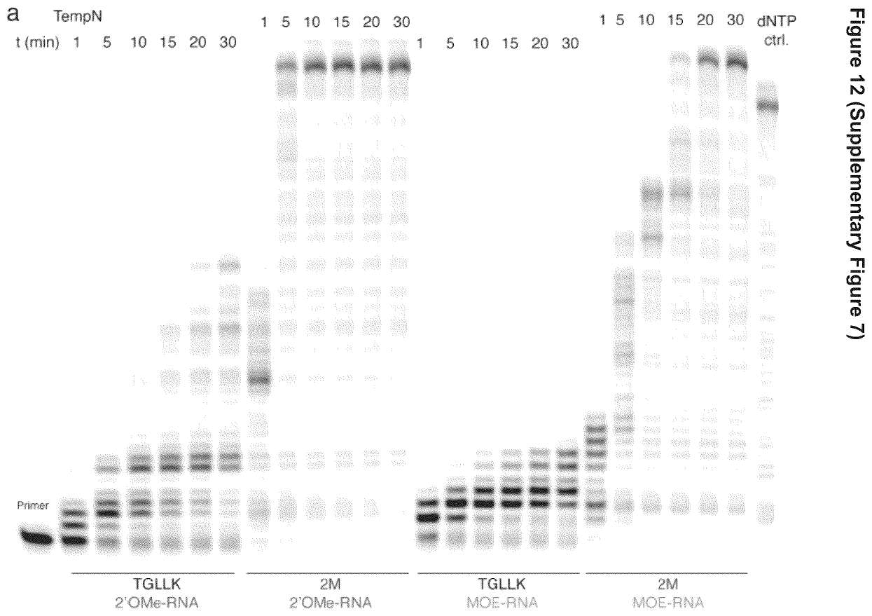

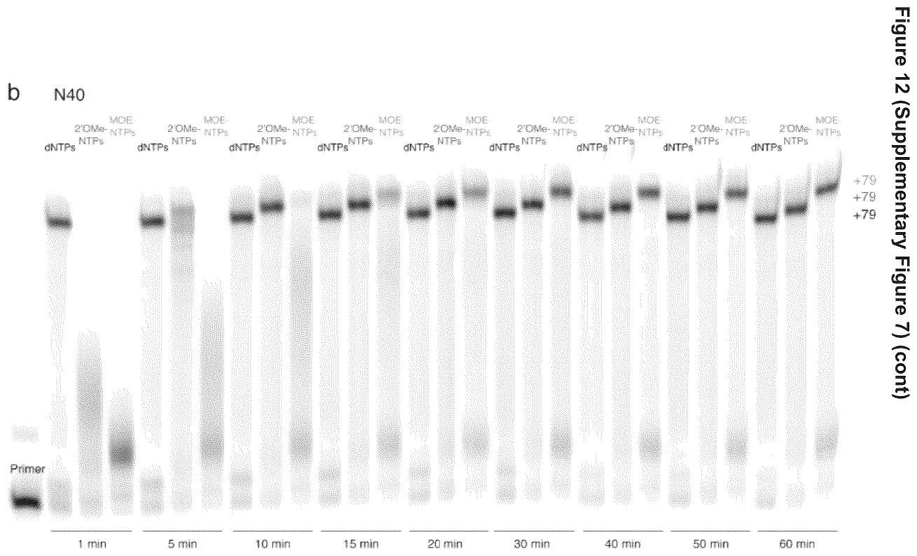

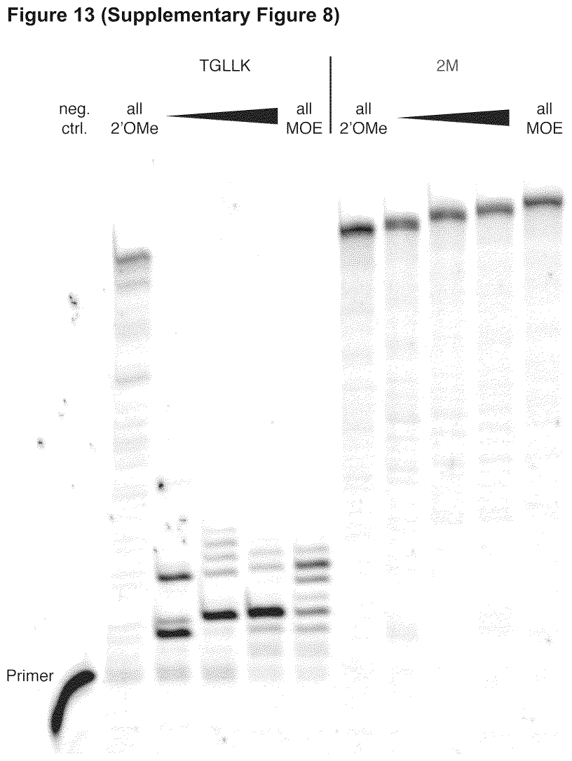

Having synthesized all four MOE-NTPs (MOE-ATP, MOE-GTP, MOE-CTP, MOE-m⁵UTP), we proceeded to test the new engineered polymerase 2M for its ability to synthesize MOE-RNA oligomers. Unlike its predecessor TGLLK, 2M (SI FIG. 7)) was able to efficiently synthesize MOE-RNA on both a model DNA template (+72 nt) and a random N₄₀ library template, and it was capable of long-range MOE-RNA synthesis of 750 nt oligomers (FIG. 3def, SI FIG. 7). The incorporation of the bulkier methoxyethyl substituents at full substitution resulted in an appreciable shift in electrophoretic mobility of MOE-oligomers compared to DNA or 2′OMe-RNA oligomers of the same length and sequence (SI FIG. 8).

MOE would be an attractive medicinal chemistry modification of RNA, 2′F-DNA or 2′OMe-RNA aptamers to modulate pharmacological properties and/or increase potency. Indeed, MOE-RNA and 2′OMe-RNA have similar conformational and helical preferences and similar base-pairing strength^{22, 27}. On the other hand, 2′-O-(2-meth-oxyethyl) groups present a significantly larger steric envelope (FIG. 3c), which might lead to steric conflicts with other groups in tightly folded structures. Nevertheless, it seemed plausible that functional mixed 2′OMe/MOE-RNA aptamers could be elaborated from previously described all-2′OMe-RNA leads. To test this, we examined conversion of a well-characterized all-2′OMe-RNA aptamer against Vascular Endothelial Growth Factor (VEGF)⁶ to all-MOE-RNA or mixed 2′OMe/MOE-RNA aptamers and tested their respective binding activity by surface plasmon resonance (SPR). SPR revealed that while the aptamer in which two out of four 2′OMe-nucleotides were substituted with MOE-nucleotides showed virtually identical affinities to VEGF compared to the all-2′OMe-RNA aptamer, the aptamer in which three of the 2′OMe-nucleotides were replaced by MOE-nucleotides still bound VEGF, albeit with reduced affinity (FIG. 4. SI Table 3).

The all-MOE aptamer seemed to have lost virtually all of its binding activity (SI FIG. 9), perhaps in part due to the use of MOE-m⁵UTP, whereas the original VEGF aptamer had been evolved using 2′OMe-U. Indeed, when we replaced 2′OMe-U with 2′OMe-m⁵U in the original aptamer, its binding affinity was reduced (SI FIG. 9). The 2′OMe/MOE-RNA aptamers described here are the first mixed-chemistry aptamers elaborated in such backbones and suggest that MOE-modified nucleic acids are capable of folding into tight three-dimensional structures with high affinity for their protein target.

### Discussion

Steric exclusion is a common determinant of enzyme and in particular polymerase specificity. This includes the “steric gate” residue found in the active site of most DNA polymerases thought to have evolved to exclude ribonucleoside triphosphates (present at much higher concentrations in the cell) from the polymerase active site in order to limit RNA incorporation into the genome. Kool and coworkers have shown that this may be a general mechanism of steric control of nucleobase pair dimension in the active site as an important component in replicative polymerase fidelity mechanisms²⁸. Steric factors are also likely implicated in post-synthetic inhibition of nascent strand extension upon incorporation of mismatches²⁹ or non-cognate nucleotides³⁰ either through direct clashes with the nascent strand polymerase interface or by altering conformational equilibria of the nascent duplex. Finally, relaxation of steric control is a successful strategy for polymerase engineering, for example in the 9° N DNA polymerase variants engineered for incorporation of bulky 3′-substituents in Illumina next generation sequencing³¹ or in engineering DNA polymerases for RNA synthesis or reverse transcription^{11, 17}.

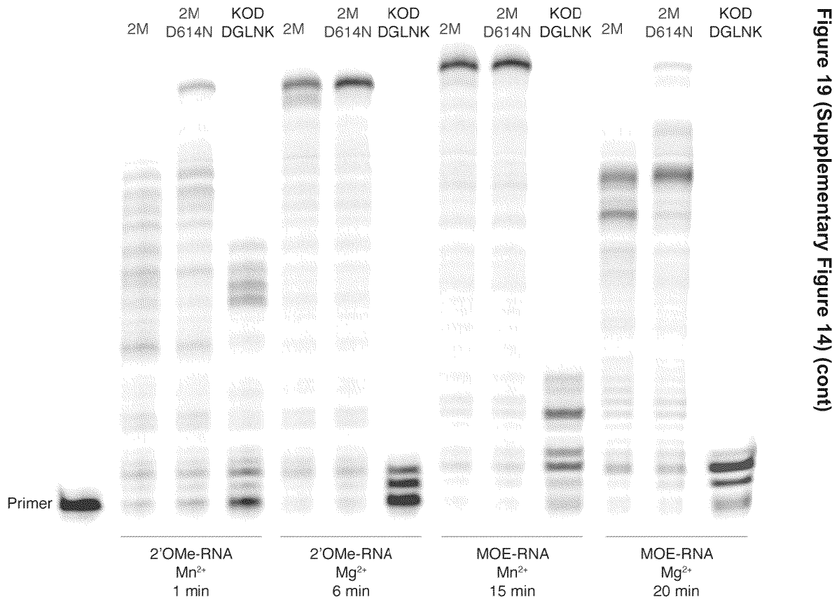

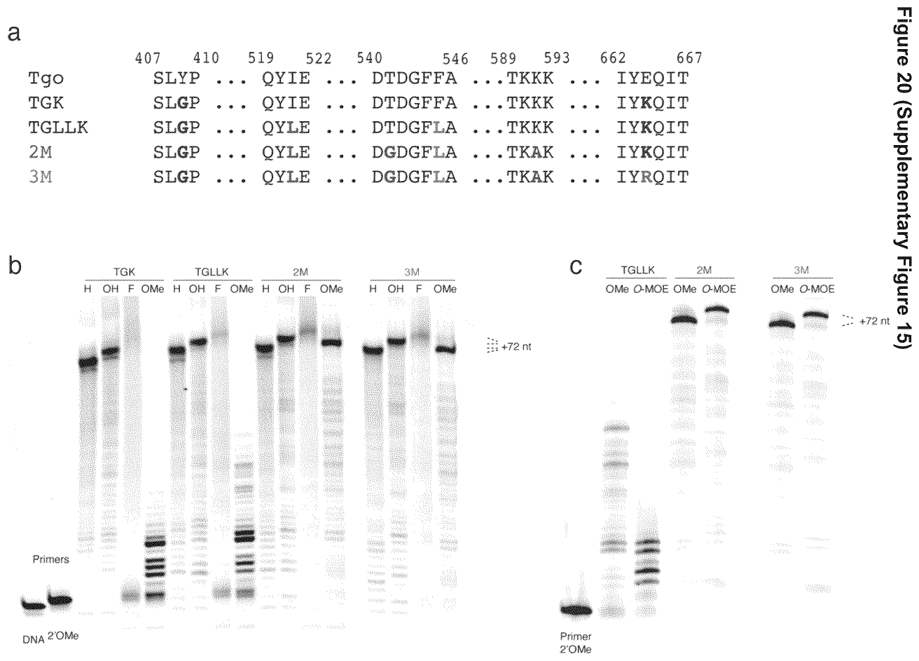

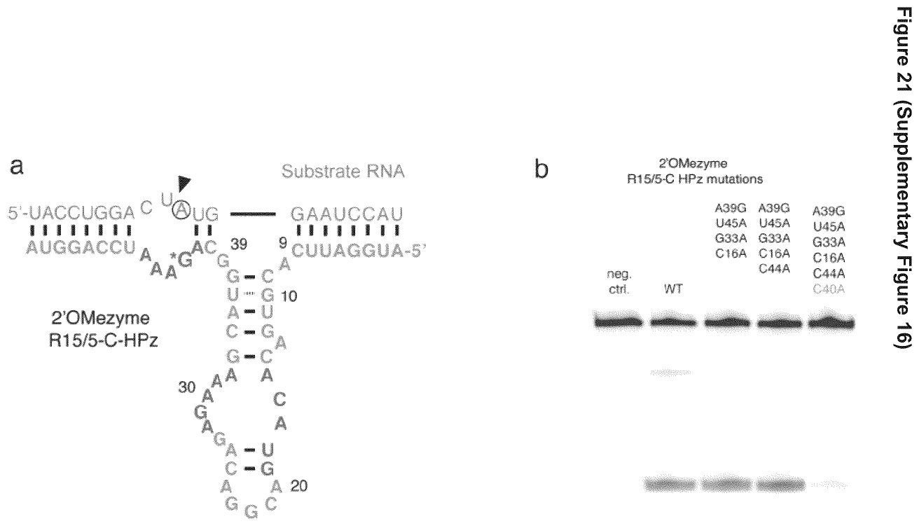

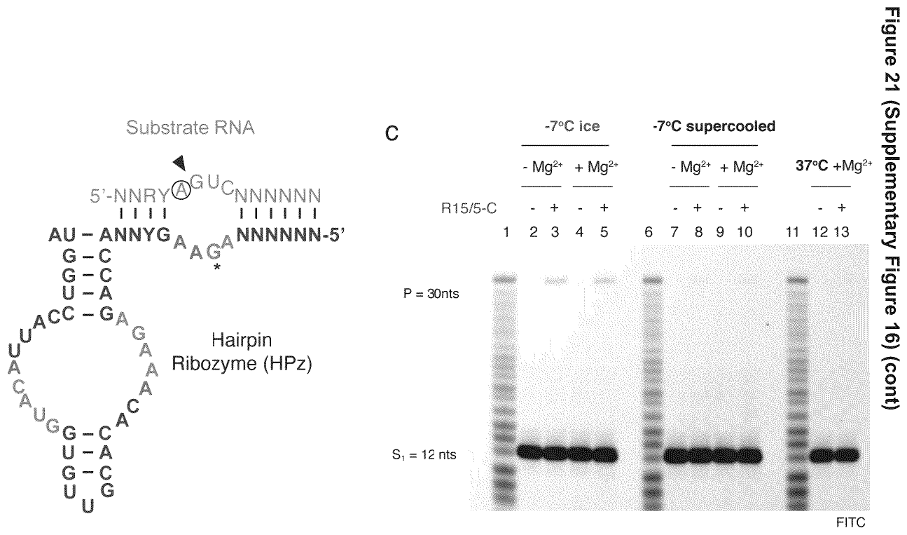

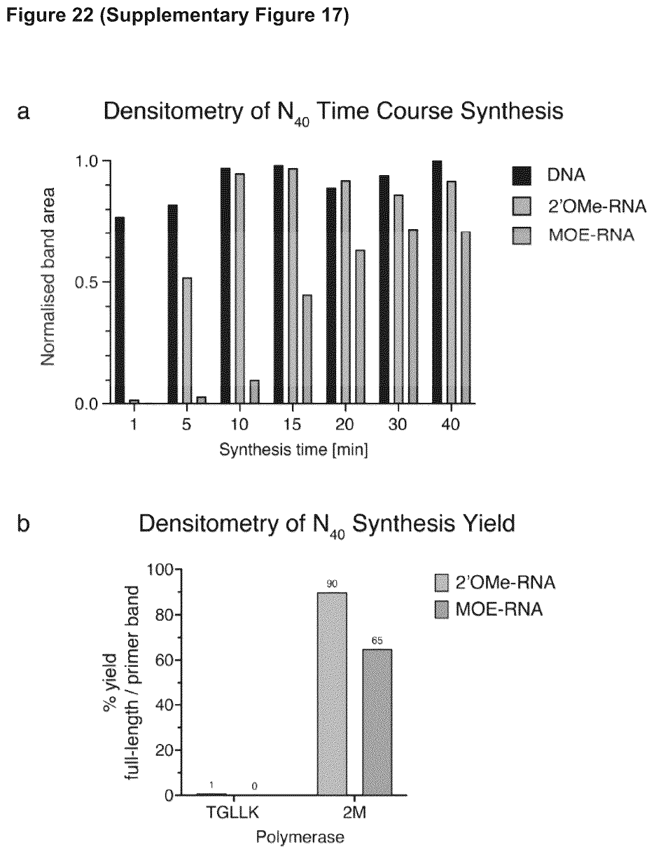

We had previously discovered key mutations in the polB family polymerase from T. gorgonarius that, in addition to the steric gate mutation (Y409G), enable efficient RNA synthesis (E664K)¹¹ and incorporation of non-cognate 2′-5′ linkages (I521L, F545L)¹⁰. The latter polymerase variant (named TGLLK) showed an increased, but still inefficient ability of 2′OMe-RNA synthesis, suggesting that aspects of the polymerase structure were still poorly adapted to 2′OMe-RNA synthesis. As RNA and 2′OMe-RNA share very similar conformational preferences, we suspected steric factors. Indeed, systematic evaluation of potential steric clashes of the polymerase with 2′-methoxy groups in the nascent strand identified a two-residue steric gate, mutation of which to less bulky side-chains (T541G, K592A) led to a dramatic increase in 2′OMe-RNA synthesis efficiency (FIG. 1) as well as for the first time enabled efficient MOE-RNA synthesis (FIG. 3) with full-length defined or random sequence (N40) products synthesized in <30 min (2′OMe-RNA, <10 min) (SI FIG. 7) despite the considerably larger steric envelope of the 2′-O-(2-methoxyethyl) group of MOE-RNA. Incorporating T541G and K592A into TGLLK led to an increase in N₄₀ synthesis yield as determined by densitometry from 1% to 90% (2′OMe-RNA) and from 0% to 65% (MOE-RNA, FIGS. 1g and 3e, SI FIG. 17).

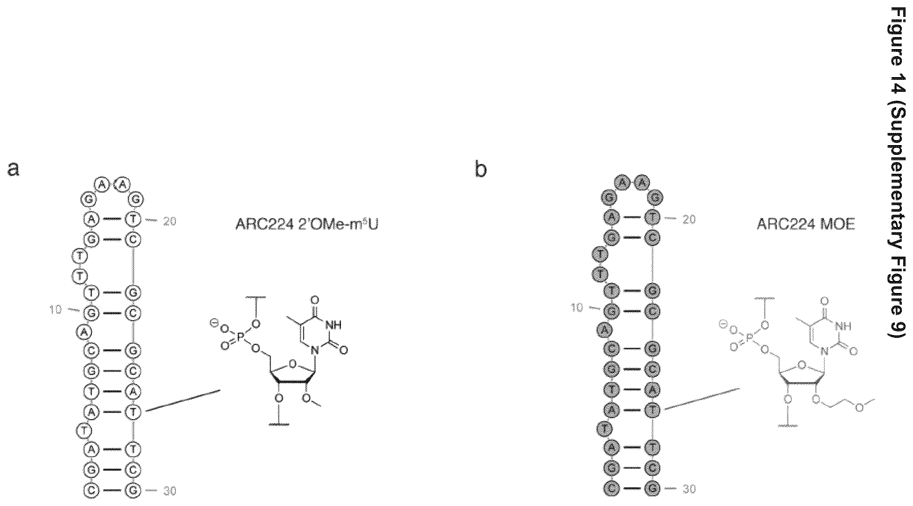

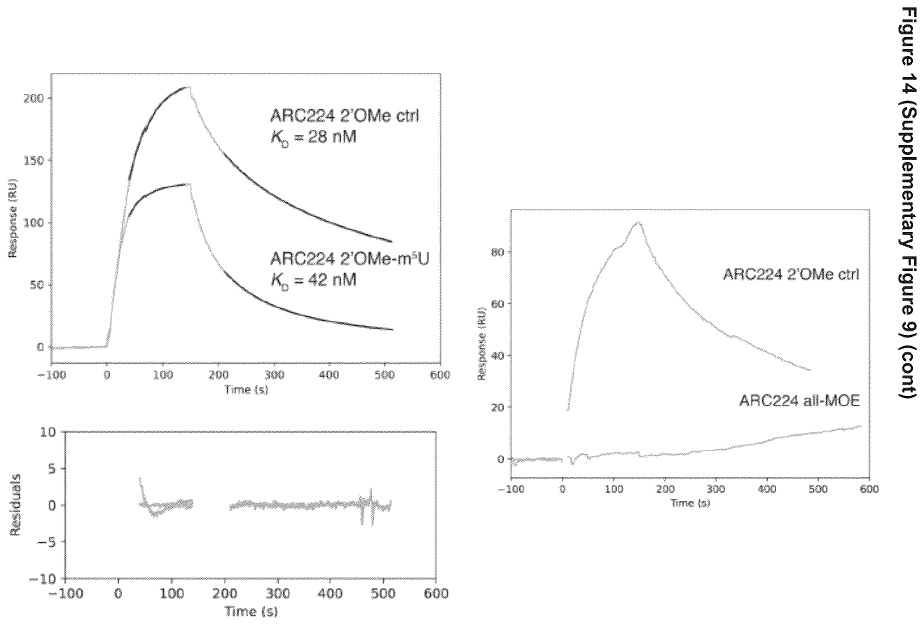

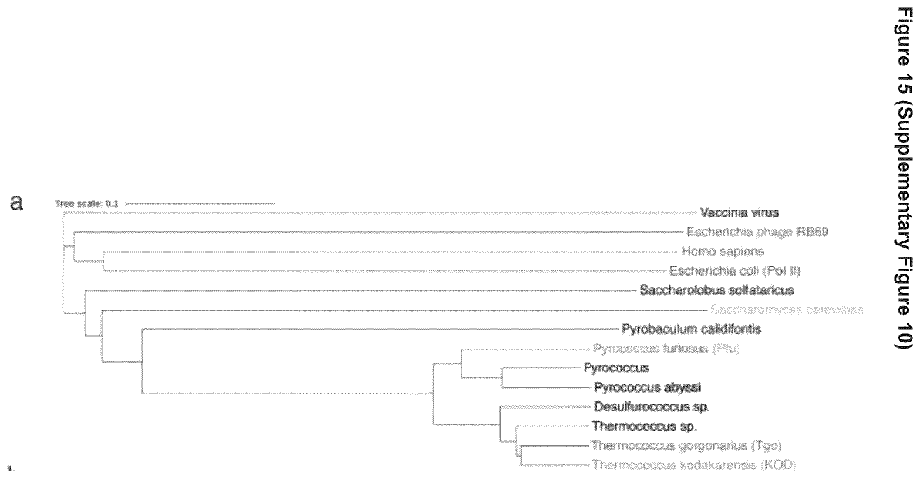

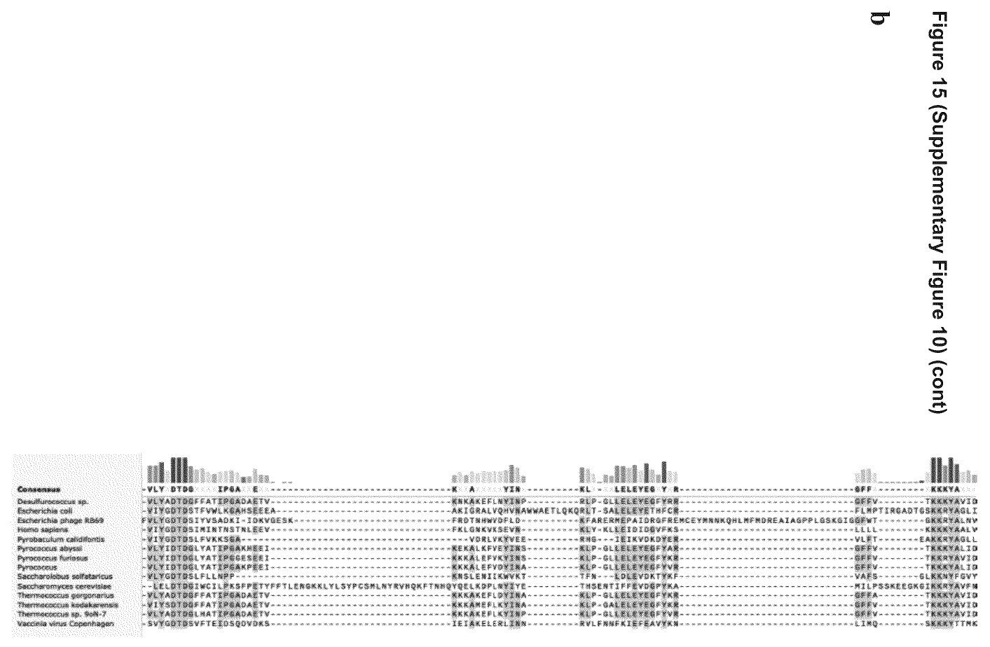

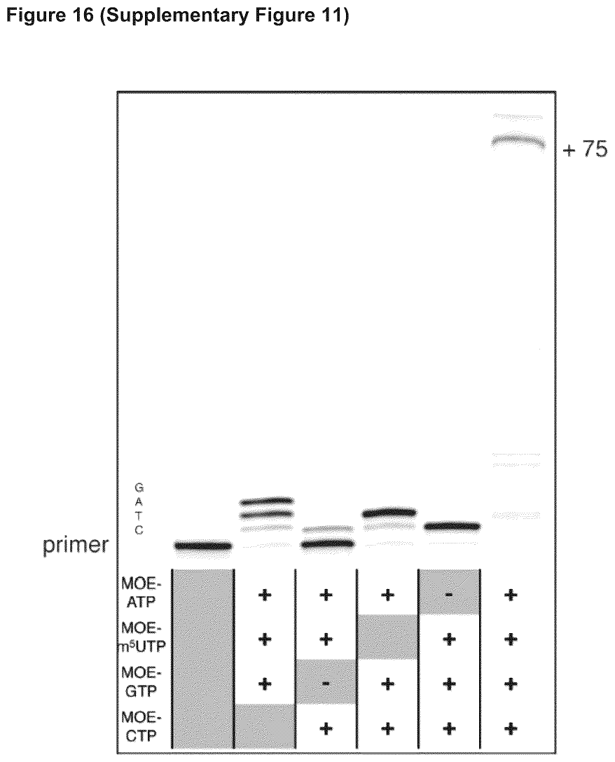

Both T541 and K592 are part of motifs (motif C³² and KxY³³, respectively) that are very highly conserved both at the sequence and at the structural level (FIG. 5, SI FIG. 10) in polB polymerases of archaeal, eukaryotic, and even viral origin³⁴. These motifs are thought to be part of a minor groove interaction motif that is involved in mismatch sensing³⁵ and previous mutation to bulky, hydrophobic side-chains was shown to enhance mismatch discrimination³⁶. Nevertheless, we find that fidelity of 2′OMe-RNA synthesis is essentially unaffected (SI Table 4) compared to parent polymerases TGK and TGLLK lacking these mutations^{10, 11}. The fidelity of MOE synthesis is currently challenging to measure due to the poor efficiency of the available MOE-RNA RT¹⁷, but a dropout assay suggests specific processing of the correct MOE-NTPs (SI FIG. 11).

According to the ternary complex structure of the closely related KOD polymerase¹², both T541 and K592 are involved in H-bonding interactions with the nascent strand 3′ end (T541, via water) and +1 (K592) nucleobases, obstructing passage of 2′-modifications (FIG. 5b). Positive epistasis of the two mutations is in congruence with structural considerations. Relieving the steric block requires mutation of both, which yields a large free volume in this critical area proximal to the catalytic site and the nascent strand large enough to also accommodate the 2′-O-methyl groups of 2′OMe-RNA (FIG. 1) and the bulky 2′-O-(2-methoxyethyl) groups of MOERNA (FIG. 3).

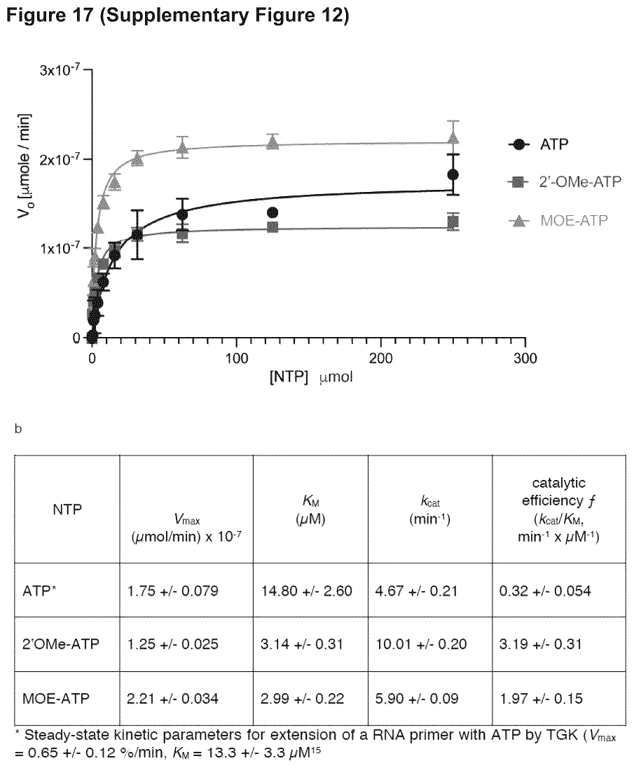

A prediction of this structural model is that this two-residue steric gate of T541 and K592 mainly enhances the efficiency of the primer 3′-end extension rather than the nucleotide incorporation step of the polymerase catalytic cycle. Indeed, 2M single nucleotide incorporation steady-state kinetic parameters for ATP (from a 2′OMe-RNA primer) (SI FIG. 12) closely match that of the parent polymerase TGK (from an RNA primer). On the other hand, while the Vₘₐₓ/k_{cat} values for incorporation of ATP, 2′OMe-ATP and MOE-ATP are essentially identical, 2M has an approximately 5-fold improved K_{M} value for both 2′OMe-ATP and MOE-ATP compared to ATP (SI FIG. 12) and compared to the parent polymerase TGK (K_{M}=13.3 μM for ATP). This may indicate that the steric gate improves the fit and positioning of 2′-modified nucleotide triphosphates into the polymerase active site, but does not accelerate the catalytic step.

While enzymatic MOE-RNA synthesis by a polymerase has not previously been described, a number of alternative engineering approaches to 2′OMe-RNA synthesis have been explored, including a variant of the closely related polB-family KOD polymerase (KOD: N210D/Y409G/A485L/D614N/E664K)⁹. While we find that 2′OMe-RNA synthesis by 2M is both more efficient (SI FIG. 14) and higher fidelity (SI Table 4) (not requiring forcing conditions such as Mn²⁺ ions), the DGLNK mutations represent an interesting alternative, non-steric strategy to enhance XNA-RNA synthesis. Starting from the same (or very similar) mutational background than 2M (including the Y409G active site steric gate, the E664K thumb subdomain mutation and the A485L “Therminator” mutation³⁷, as well as a mutation (N210D) to inactivate the 3′-5′ exonuclease domain, DGLNK also comprises a critical D614N mutation in the thumb subdomain, which removes of a negative charge in proximity to the phosphodiester backbone of the nascent strand. This is highly reminiscent of the previously described Tgo: E664K mutation that was found to enable efficient RNA synthesis by expanding the positively charged polymerase interaction surface and enhancing affinity for the primer-template duplex. While not demonstrated for DGLNK, it is plausible that the D614N mutation, which further reduces negative charge potential at the polymerase-nascent strand interface, also enhances affinity of the polymerase for the primer-template duplex. At the same time, our original model had identified D614 as a potential steric clash with the nascent strand methoxy groups, but our screen had not identified any strong positive effect on 2′OMe-RNA synthesis as an isolated mutation. Nevertheless, we re-examined the D614N mutation in the context of 2M (2MN; 2M: D614N) and found a small enhancement of 2′OMe-RNA and to a lesser extent MOE-RNA synthesis by 2MN (SI FIG. 14).

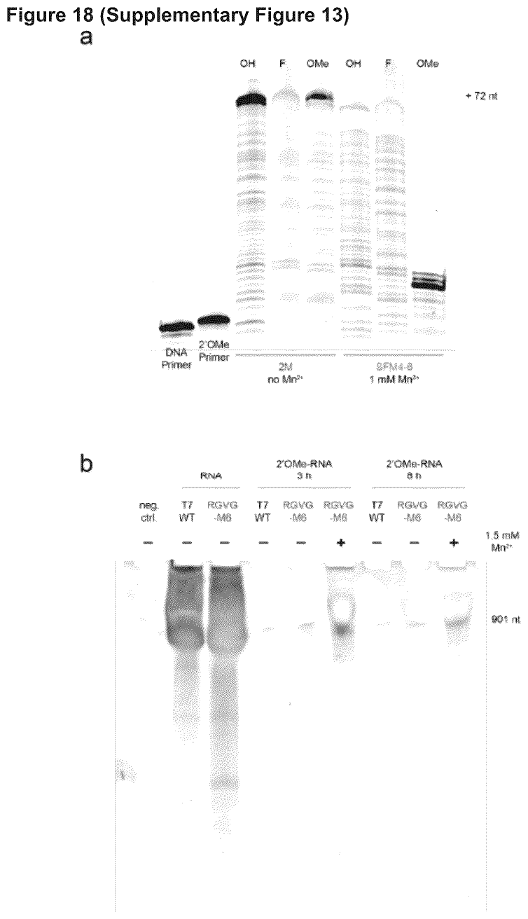

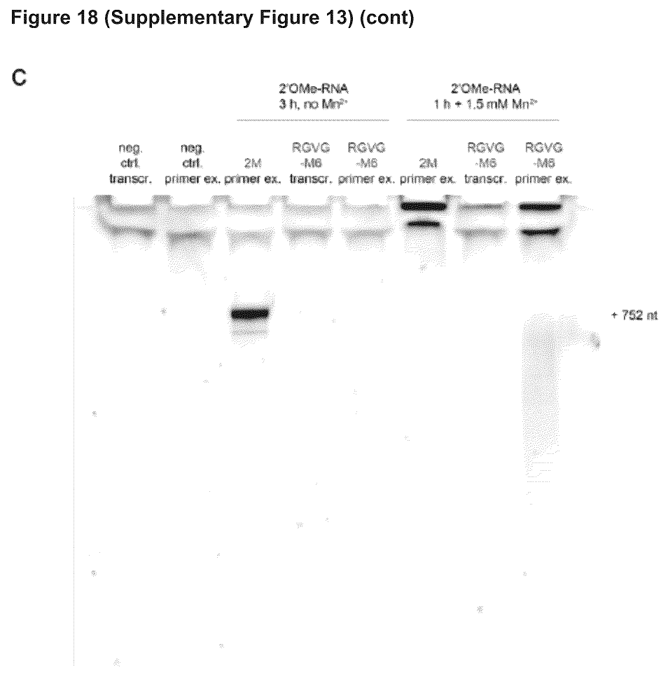

We also evaluated two other previously published polymerases, T7 RNA polymerase variant RGVG-M6 (T7: P266L, S430P, N433T, E593G, S633P, Y639V, V685A, H784G, F849I, F880Y) and Taq polymerase Stoffel fragment variant SFM4-6 (Taq SF: I614E, E615G, D655N, L657M, E681K, E742N, M747R), that had been reported to have 2′OMe-RNA synthesis activity. However, compared to 2M, the 2′OMe-RNA synthesis activity appeared to be modest in both cases and dependent on forcing conditions such as the presence of high concentrations of Mn2+ ions (SI FIG. 13).

Finally, as our initial screen also indicated that TGLLK: T541G, K664R (SI FIG. 1) also exhibited a (smaller) increase in 2′OMe-RNA synthesis efficiency compared to the single mutant T541G, we introduced K664R into the 2M polymerase, yielding TGLLK: T541G, K592A, K664R (henceforth named 3M). However, polymerases 2M and 3M exhibited virtually identical synthesis activity, full-length yield, and stalling pattern (SI FIG. 15).

Together with the discovery of a more efficient 2′OMe-RNA RT¹⁷, 2M has opened the door for more ambitious in vitro evolution experiments, including the discovery of the first 2′OMezymes. Unlike 2′OMe-RNA aptamers, no 2′OMezymes had previously been described, presumably due to the fact that catalysts generally appear to be more sparsely distributed in nucleic acid sequence space³⁸. The RNA endonuclease 2′OMezymes R15/5-K and -C characterized herein differ in interesting ways from other RNA endonuclease DNA- and XNAzymes described. While highly specific, their maximal catalytic turnover is modest, possibly due to overly tight binding of the RNA substrate by 2′OMe-RNA, leading to product inhibition and/or a high proportion of 2′OMezymes trapped in non-catalytic conformations. However, unlike for example the canonical 10-23 DNAzyme, or some XNAzymes, 2′OMezymes retain much of their catalytic activity at low, physiologically relevant Mg²⁺ concentrations. This suggests that unlike the above, the 2′OMezymes are likely not obligate metalloenzymes, but may instead rely on acid-base catalysis akin to the classic hairpin ribozyme (Hpz). Intriguingly, the 2′OMezymes—despite lacking sequence homology—share some striking secondary structure and sequence segment similarities with the hairpin ribozyme³⁹ (albeit with the hairpin and cleavage sites reversed) (SI FIG. 16). Like the Hpz, the 2′OMezymes also have the capacity to catalyze RNA ligation at low temperatures (SI FIG. 16) and exhibit activity in the absence of Mg²⁺ (SI FIG. 2). Consistent with this, mutations that increase the sequence identity with HPz are mostly benign (SI FIG. 16).

The 2M polymerase for the first time enables the templated enzymatic synthesis of MOE-RNA, a nucleic acid modification of great interest in nucleic acid therapeutics due to its unusual structural and pharmacological properties and extraordinary biostability, which have driven its application in FDA-approved ASO drugs². This makes MOE a desirable medicinal chemistry modification of existing 2′OMe-RNA aptamers. In the case of an anti-VEGF 2′OMe-RNA aptamer⁶, chimeric versions in which two or three of the 2′OMe-nucleotides were replaced by MOE-nucleotides could be readily elaborated and showed identical or slightly reduced binding affinities for VEGF, respectively (FIG. 4), although full substitution of 2′OMe- with MOE RNA abolished binding activity in this aptamer (SI FIG. 9).

In conclusion, our work underlines the importance of steric control in polymerase substrate specificity. Discovery of the new two-residue nascent strand steric gate complements the classic active site steric gate in excluding 2′-modified nucleic acids from incorporation into the nascent strand and unlocks enzymatic synthesis of nucleic acid oligomers bearing bulky 2′-substituents. This has enabled the efficient synthesis and evolution of 2′OMezymes as well as MOE-RNA synthesis and elaboration of mixed 2′OMe-/MOE-RNA aptamers. We envisage a range of applications including the stereospecific synthesis of phosphorothioate (aPS)-MOE-RNA oligomers and the rapid iteration of variant aptamer and ASO sequences and chemistries towards enhanced potency.

### Materials & Methods

### Nucleotides and Oligonucleotides

Triphosphates of 2′OMe-RNA (2′OMe-NTPs; 2′OMe-ATP, 2′OMe-CTP, 2′OMe-GTP, 2′OMe-UTP) were obtained from Jena Biosciences (Germany) and DNA (Illustra dNTPs) from GE Life Sciences (USA). Oligonucleotides were synthesized by Integrated DNA Technologies (Belgium) or Merck/MilliporeSigma (Germany). A gBlock encoding SFM4-6 was synthesized by Integrated DNA Technologies (Belgium) and gene synthesis of pET28a(+)-His6-RGVG-M6 was performed by GenScript Biotech (UK).

### Synthesis of 2′-O-MOE-NTPs

### 1. General Synthetic Information for 2′-O-MOE-NTP Synthesis

All reagents and solvents were purchased from commercial sources and used as obtained. Moisture-sensitive reactions were carried out in vacuum-dried glassware under a nitrogen atmosphere. ¹H, ¹³C, and ³¹P NMR spectra were recorded on a Bruker Avance 300, 500, or 600 MHz spectrometer using tetramethylsilane as internal standard or by referencing to the residual solvent signal [D₂O (d=4.79 ppm ¹H NMR)]. Coupling constants are reported in Hertz (Hz) and were directly obtained from the spectra. NMR splitting patterns are designated as s (singlet), d (doublet), t (triplet), q (quartet), and m (multiplet). High-resolution mass spectra (HRMS) were obtained on a quadruple orthogonal acceleration time-of-flight mass spectrometer (Synapt G2 HDMS, Waters, Milford, MA). Samples were infused at 3 μL/min, and spectra were obtained in negative ionization mode with a resolution of 15 000 FWHM using leucine enkephalin as the lock mass. Pre-coated aluminium sheets (254 nm) were used for thin layer chromatography (TLC). Products were purified by preparative HPLC ionexchange chromatography (SOURCE 15Q) using 0.1 M/1 M TEAB buffer as eluent followed by preparative ion-paired reversed-phase HPLC (Phenomenex Gemini 110A, C18, 10 μm, 21.2 mm×250 mm) using 0.1 M TEAB buffer/0.05 M TEAB in acetonitrile/water 1:1 (v/v) as elution system.

2. General Procedure for Conversion of Triethylammonium Salts into Sodium Salts

Triethylammonium nucleoside triphosphate (4-7 mg) was lyophilised in a plastic tube. The compound was dissolved in methanol (500 μL) and NaClO₄ (0.1 M in acetone, 3 mL) was added quickly. This led to precipitation of the sodium nucleoside triphosphate salt. The tube was centrifuged and the supernatant discarded. The pellet was washed twice with acetone and then dried under vacuum.

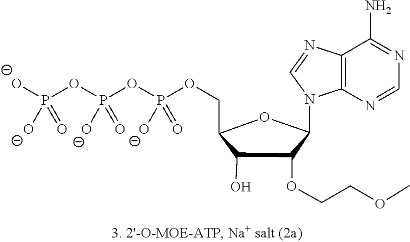

All solid reagents were weighed and dried in the reaction flasks under vacuum in a desiccator overnight. Under a nitrogen atmosphere, 2′-O-(2-methoxyethyl)adenosine (50 mg, 0.15 mmol, 1.0 eq.) and proton sponge (66 mg, 0.30 mmol, 2.0 eq.) were dissolved in trimethyl phosphate (4 mL). At −15° C., phosphoryl oxychloride (22 μL, 0.24 mmol, 1.5 eq.) was added and the reaction mixture was stirred at −15° C. for 2 h. After reaction monitoring with analytical anion-exchange HPLC, the reaction mixture was allowed to warm to room temperature. Tris(tetrabutylammonium) hydrogen pyrophosphate (554 mg, 0.62 mmol, 4.0 eq.) and tributylamine (370 μL, 1.60 mmol, 10.0 eq.) were dissolved in DMF (1 mL) and this solution was added to the reaction mixture. The mixture was stirred at room temperature for 30 min. Triethylammonium bicarbonate (TEAB) buffer (1 M, 20 mL) was added to quench the reaction and the reaction mixture was extracted with diisopropylether (20 mL). The aqueous phase was lyophilised. The reaction mixture was purified via preparative anion-exchange HPLC with a 0.1 M TEAB-1 M TEAB gradient, followed by ion-paired reversed-phase HPLC with a 0.1 M TEAB-0.05 M TEAB in acetonitrile/water 1:1 (v/v) gradient. The product was obtained as the triethylammonium salt (31.0 mg, 20.8%) as a white powder. For analytical purposes, the triethylammonium salt was converted into the sodium salt. 1H NMR (600 MHz, D₂O): δ (ppm)=8.54 (s, 1H), 8.27 (s, 1H), 6.20 (d, J=6.3 Hz, 1H), 4.72-4.69 (m, 1H), 4.63-4.60 (m, 1H), 4.43-4.40 (m, 1H), 4.31-4.26 (m, 1H), 4.25-4.19 (m, 1H), 3.87-3.82 (m, 1H), 3.74-3.70 (m, 1H), 3.54-3.46 (m, 2H), 3.15 (s, 3H).

¹³C NMR (151 MHz, D₂O): δ (ppm)=155.62, 152.84, 149.12, 139.98, 118.56, 85.32, 84.54, 82.12, 70.95, 69.53, 69.18, 65.25, 57.80.

³¹P NMR (202 MHz, D₂O): δ (ppm)=−9.39-−10.45 (m, 1P), −11.31 (d, J=18.7 Hz, 1P), −22.33-−23.46 (m, 1P).

ESI-MS calculated [M-H]−: m/z=564.03032; found [M-H]−: m/z=564.0279 (10%).

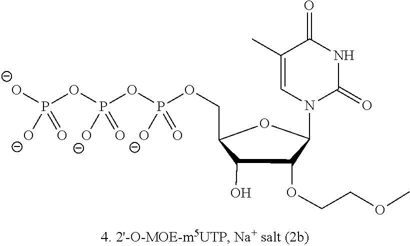

All solid reagents were weighed and dried in the reaction flasks under vacuum in a desiccator overnight. Under a nitrogen atmosphere, 2′-O-(2-methoxyethyl)-5-methyluridine (50 mg, 0.16 mmol, 1.0 eq.) and proton sponge (68 mg, 0.32 mmol, 2.0 eq.) were dissolved in trimethyl phosphate (4 mL). At −15° C., phosphoryl oxychloride (22 μL, 0.24 mmol, 1.5 eq.) was added and the reaction mixture was stirred at −15° C. for 2 h. After reaction monitoring with analytical anion-exchange HPLC, the reaction mixture was allowed to warm to room temperature. Tris(tetrabutylammonium) hydrogen pyrophosphate (571 mg, 0.63 mmol, 4.0 eq.) and tributylamine (376 μL, 1.58 mmol, 10.0 eq.) were dissolved in DMF (1 mL) and this solution was added to the reaction mixture. The mixture was stirred at room temperature for 30 min. Triethylammonium bicarbonate (TEAB) buffer (1 M, 20 mL) was added to quench the reaction and the reaction mixture was extracted with diisopropylether (20 mL). The aqueous phase was lyophilised. The reaction mixture was purified via preparative anion-exchange HPLC with a 0.1 M TEAB-1 M TEAB gradient, followed by ion-paired reversed-phase HPLC with a 0.1 M TEAB-0.05 M TEAB in acetonitrile/water 1:1 (v/v) gradient. The product was obtained as the triethylammonium salt (42.5 mg, 28.0%) as a white powder. For analytical purposes, the triethylammonium salt was converted into the sodium salt.

¹H NMR (500 MHz, D₂O): δ (ppm)=7.80 (s, 1H), 6.06 (d, J=5.4 Hz, 1H), 4.57-4.53 (m, 1H), 4.31-4.28 (m, 1H), 4.28-4.22 (m, 3H), 3.84 (q, J=4.2 Hz, 2H), 3.63 (t, J=4.4 Hz, 2H), 3.35 (s, 3H), 1.96 (s, 3H).

¹³C NMR (126 MHz, D₂O): δ (ppm)=166.49, 151.77, 137.01, 111.89, 86.51, 83.48, 81.20, 71.05, 69.36, 68.36, 64.82, 58.00, 11.59.

³¹P NMR (202 MHz, D₂O): δ (ppm)=−9.72-−10.87 (m, 1P), −11.64 (d, J=18.8 Hz, 1P), −22.54-−23.50 (m, 1P). ESI-MS calculated [M-H]−: m/z=555.01875; found [M-H]−: m/z=555.0176 (10%).

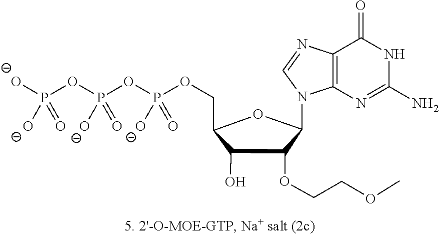

All solid reagents were weighed and dried in the reaction flasks under vacuum in a desiccator overnight. Under a nitrogen atmosphere, 2′-O-(2-methoxyethyl)guanosine (50 mg, 0.15 mmol, 1.0 eq.) and proton sponge (63 mg, 0.29 mmol, 2.0 eq.) were dissolved in trimethyl phosphate (4 mL). At −15° C., phosphoryl oxychloride (21 μL, 0.22 mmol, 1.5 eq.) was added and the reaction mixture was stirred at −15° C. for 2 h. After reaction monitoring with analytical anion-exchange HPLC, more phosphoryl oxychloride (21 μL, 0.22 mmol, 1.5 eq.) was added and the reaction mixture was stirred at −15° C. for another 2 h. This was repeated one more time with a third addition of phosphoryl oxychloride (21 μL, 0.22 mmol, 1.5 eq.). After reaction monitoring with analytical anion-exchange HPLC, the reaction mixture was allowed to warm to room temperature. Tris(tetrabutylammonium) hydrogen pyrophosphate (1058 mg, 1.18 mmol, 8.0 eq.) and tributylamine (696 μL, 2.92 mmol, 20.0 eq.) were dissolved in DMF (2 mL) and this solution was added to the reaction mixture. The mixture was stirred at room temperature for 30 min. Triethylammonium bicarbonate (TEAB) buffer (1 M, 20 mL) was added to quench the reaction and the reaction mixture was extracted with diisopropylether (20 mL). The aqueous phase was lyophilised. The reaction mixture was purified via preparative anion-exchange HPLC with a 0.1 M TEAB-1 M TEAB gradient, followed by ion-paired reversed-phase HPLC with a 0.1 M TEAB-0.05 M TEAB in acetonitrile/water 1:1 (v/v) gradient. The product was obtained as the triethylammonium salt (24.5 mg, 17.0%) as a white powder. For analytical purposes, the triethylammonium salt was converted into the sodium salt.

¹H NMR (600 MHz, D₂O): δ (ppm)=8.11 (s, 1H), 5.97 (d, J=6.1 Hz, 1H), 4.71-4.67 (m, 2H), 4.38-4.35 (m, 1H), 4.29-4.19 (m, 2H), 3.86-3.82 (m, 1H), 3.74-3.70 (m, 1H), 3.55-3.48 (m, 2H), 3.21 (s, 3H).

¹³C NMR (151 MHz, D₂O): δ (ppm)=159.01, 153.87, 151.80, 137.99, 116.24, 85.52, 84.35, 80.91, 70.91, 69.40, 69.00, 65.21, 57.85.

³¹P NMR (202 MHz, D₂O): δ (ppm)=−9.92 (d, J=16.3 Hz, 1P), −11.31 (d, J=18.8 Hz, 1P), −22.85 (t, J=19.1 Hz, 1P).

ESI-MS calculated [M-H]−: m/z=580.02523; found [M-H]−: m/z=580.0270 (11%).

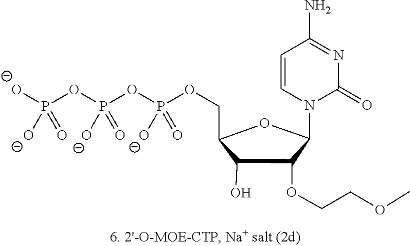

All solid reagents were weighed and dried in the reaction flasks under vacuum in a desiccator overnight. Under a nitrogen atmosphere, 2′-O-(2-methoxyethyl)cytidine (50 mg, 0.17 mmol, 1.0 eq.) and proton sponge (71 mg, 0.33 mmol, 2.0 eq.) were dissolved in trimethyl phosphate (4 mL). At −15° C., phosphoryl oxychloride (23 μL, 0.25 mmol, 1.5 eq.) was added and the reaction mixture was stirred at −15° C. for 2 h. After reaction monitoring with analytical anion-exchange HPLC, more phosphoryl oxychloride (23 μL, 0.25 mmol, 1.5 eq.) was added and the reaction mixture was stirred at −15° C. for another 2 h. After reaction monitoring with analytical anionexchange HPLC, the reaction mixture was allowed to warm to room temperature. Tris(tetrabutylammonium) hydrogen pyrophosphate (1198 mg, 1.32 mmol, 8.0 eq.) and tributylamine (788 μL, 3.32 mmol, 20.0 eq.) were dissolved in DMF (2 mL) and this solution was added to the reaction mixture. The mixture was stirred at roomtemperature for 30 min. Triethylammonium bicarbonate (TEAB) buffer (1 M, 20 mL) was added to quench the reaction and the reaction mixture was extracted with diisopropylether (20 mL). The aqueous phase was lyophilised. The reaction mixture was purified via preparative anion-exchange HPLC with a 0.1 M TEAB-1 M TEAB gradient, followed by ion-paired reversed-phase HPLC with a 0.1 M TEAB-0.05 M TEAB in acetonitrile/water 1:1 (v/v) gradient. The product was obtained as the triethylammonium salt (20.0 mg, 12.7%) as a white powder. For analytical purposes, the triethylammonium salt was converted into the sodium salt.

¹H NMR (600 MHz, D₂O): δ (ppm)=8.01 (d, J=7.6 Hz, 1H), 6.15 (d, J=7.6 Hz, 1H), 6.06 (d, J=4.0 Hz, 1H), 4.48 (t, J=5.3 Hz, 1H), 4.33-4.23 (m, 3H), 4.15-4.12 (m, 1H), 3.91 (dt, J=11.6, 4.5 Hz, 1H), 3.84 (dt, J=11.7, 4.2 Hz, 1H), 3.64 (t, J=4.4 Hz, 2H), 3.36 (s, 3H).

¹³C NMR (151 MHz, D₂O): δ (ppm)=166.12, 157.45, 141.44, 96.56, 87.59, 82.72, 82.01, 71.14, 69.36, 67.96, 64.28, 58.00.

³¹P NMR (202 MHz, D₂O): δ (ppm)=−8.66-−9.72 (m, 1P), −11.35 (d, J=18.8 Hz, 1P), −22.74 (t, J=18.4 Hz, 1P).

ESI-MS calculated [M-H]−: m/z=540.01908; found [M-H]−: m/z=540.0197 (65%) (recorded as TEA salt).

### Polymerase Models and Rational Choice of Mutagenesis Sites

For construction of the mini-libraries introducing single mutants at specific polymerase residues, we used the ternary crystal structure of the closed form of Thermococcus kodakarensis KOD1 DNA polymerase in complex with a DNA primer-template duplex and an incoming dATP at the active site (PDB ID 5OMF)1, as this is a close B-family homologue of the Thermococcus gorgonarius polymerase mutants used in this study. The crystal structure was loaded in Pymol and appropriate 2′-hydrogen atoms of primer nucleotides were manually replaced by oxygen atoms with Pymol's “build” functionality. The hydrogen atoms on the newly introduced 2-hydroxyl moieties were then replaced in the same manner by methyl groups. The added dihedral angles were adjusted manually to 71° (gauche conformation)^{2, 3}. This model served as a structural guide to calculate distances from polymerase residues to the introduced primer 2′-O-methyl carbon atoms and identify sites of steric clashes. These were targeted for site-saturation mutagenesis to relieve the steric hindrance and increase polymerase processivity on 2′OMe-RNA.

### Cloning of Expression Constructs and Site-Saturation Mutagenesis

Inverse PCR (iPCR) was carried out using overlapping forward and reverse primers introducing a BsaI restriction site (see Supplementary Table 1) on pASK75 plasmid⁴ coding for Thermococcus gorgonarius (Tgo) polymerase mutant TGLLK (Tgo: V93Q, D141A, E143A, Y409G, A485L, I521L, F545L, E664K)⁵ as the parent plasmid. The cloning primers for site-saturation mutagenesis contained degenerate NNS codons (N for all bases, S for G and C) introducing mini-libraries of 32 codons coding for all 20 amino acids on a single residue (see Supplementary Table 1).

iPCR reactions were carried out with polymerase Q5 (New England Biolabs, NEB) with forward and reverse primers (0.5 μM each) and dNTPs (200 μM each) on 20 ng DNA template. The iPCR reactions were incubated in the thermocycler with the following programme: 98° C., 30 s; 30 cycles of (98° C., 10 s; 50-72° C., 30 s; 72° C., 3 min); 72° C., 3 min. iPCR products were purified using the PCR Purification Kit (Qiagen). The products were restricted by BsaI and DpnI (NEB) and purified on an agarose gel if necessary. Products were ligated by T4 DNA ligase and purified by another clean-up kit (Bioline). The cloned constructs were transformed into chemically or electrocompetent E. coli 10-β cells (NEB) or E. coli BL21 CodonPlus-RIL cells (Agilent) and plated on TYE agar plates supplemented with the appropriate antibiotics.

### Primer Extension Reactions

Analytical primer extension reactions were carried out in 1× Thermopol buffer (NEB) supplemented with MgSO₄ (4 mM). Primer (100 nM) was extended on a template (200 nM) with appropriate nucleoside triphosphates (125-250 μM each) by purified polymerase (10-100 μg/mL) in a 10-μL reaction volume. Reactions were carried out at 65° C. Primer extension products were analysed via urea-PAGE. All extensions with MOE-NTPs on defined-sequence template TempNpure required post-synthesis template capture with a ten-fold excess of antisense template, Turbo DNase (Invitrogen) treatment, subsequent Proteinase K (NEB) treatment, and loading on the urea-PAGE gel with a ten-fold excess of antisense template. Primer extensions with MOE-NTPs on template sfGFP required polymerase concentrations of 500 μg/mL.

### Enzyme-Linked Oligonucleotide Assay (ELONA) Polymerase Activity Assay (PAA)

Site-saturation mutagenised polymerase mini-libraries were transformed in E. coli 10-β cells and plated on TYE agar plates supplemented with ampicillin. For every single mutant mini-library, 2×94 clones were manually picked from the agar plates and used to inoculate 2×94 liquid starting cultures of 1 mL 2×TY supplemented with ampicillin (100 μg/mL) in 96-deep well plates (Nunc) alongside two control wells per plate with parent polymerase TGLLK. The cultures were grown at 37° C. overnight. The next day, 100 μL of each culture was used to inoculate a new 1-mL culture on a new plate and the cultures were allowed to grow at 37° C. until they reached mid-log phase. Protein expression was then induced with anhydrotetracycline at 200 μg/L and carried out at 37° C. for 2 h. The cultures were stored at 4° C. overnight. The cells were harvested by centrifugation and then resuspended in 100 μL Thermopol buffer. The cells were transferred to a 200-μL 96-well plate and lysed at 75° C. for 30 min. Lysed cells were cooled in an ice-water bath and the lysates were cleared by centrifugation at 4° C. The cleared lysates were transferred to a new 200-μL 96-well plate and stored at 4° C.

Primer extension reactions were carried out in 1× Thermopol buffer (NEB) supplemented with MgSO₄ (4 mM). Biotinylated primer FD (100 nM) was extended on template TempNpure (200 nM) with 2′-O-methylribonucleoside triphosphates (125 μM each) by polymerase mutants in whole-cell lysate in a 10-μL reaction volume. Reactions were carried out at 65° C.

The biotinylated primer extension products were diluted in PBS supplemented with 0.1% (v/v) Tween 20 (PBST) and bound on streptavidin-coated plates (Roche) for 1 h at room temperature. After every incubation step, the respective supernatant was discarded. Hybridised template was then removed by two 1-min denaturation steps with 0.1 M NaOH. After a neutralisation step with PBST, a digoxigenin labelled oligonucleotide probe (DIGN25, 60 nM in PBST) was applied for 1 h, which hybridised to efficiently elongated primers only, exhibiting increasing affinity the longer the extension product was.

After three washing steps with PBST, an anti-digoxigenin antibody fragment bound to horseradish peroxidase (1:3,000 dilution in PBST, Roche) was bound on the plates for 1 h. After four PBST washes, the assay was developed by the addition of 3,3′,5,5′-tetramethylbenzidine (TMB, 1-Step Ultra TMB-ELISA, Thermo) and incubation until the blue colour formation was complete (judged by TGLLK control wells). The enzymatic reaction was stopped by the addition of 1 M H₂SO₄, which lead to a yellow colour switch. Absorbance was read on a plate reader at 450 nm.

Screen hits were mini-prepped and sequenced, and polymerase activity was verified with extension reactions of a fluorescently labelled primer FD as described above, where the amount of lysate added was adjusted by SDS-PAGE analysis and normalisation based on the polymerase band intensities. Primer extension products were analysed via urea-PAGE.

### Expression and Purification of Polymerases

Polymerase expression and purification was essentially performed as described previously⁶. Briefly, a starting culture of E. coli BL21 CodonPlus-RIL cells (Agilent) was inoculated from a single colony and grown in 2×TY media supplemented with ampicillin (100 μg/mL) and chloramphenicol (25 μg/mL) at 37° C. overnight. This was used to inoculate 30 mL (small scale) or 1 L (large scale) of the same media the next day. The culture was grown until mid-log phase and expression was induced with anhydrotetracycline at 200 μg/L for 4 h at 37° C. After storage at 4° C. overnight, harvested cells were lysed at 75° C. for 30 min and lysates were cleared by centrifugation. His-tagged polymerases were benchtop-purified via gravity flow on Ni-NTA agarose resin (Qiagen) while non-His-tagged polymerases were benchtop-purified via gravity flow on DEAE Sepharose fast flow anion exchange resin (GE Healthcare). Then eluted fractions were loaded onto a 16/10 Hi-Prep Heparin FF column (Cytiva Life Sciences) and eluted at 0.5-0.8 M NaCl. Appropriate fractions were filter-dialysed (Amicon Ultra Centrifugal Filters, Millipore) into 2× polymerase storage buffer (1M KCl, 2 mM 290 EDTA, 20 mM Tris pH 7.4) and stored in 50% glycerol at −20° C.

### Synthesis of Long Fluorophore-Labelled RNA

Human cDNA clones for KRAS (transcript variant b, accession no. NM_004985) and CTNNB1 (transcript variant 1, accession no. NM_001904) in plasmids pCMV6-XL6 (SP6 promoter) (cat. no. SC109374) and pCMV6-XL5 (T7 promoter) (cat. no. SC107921), respectively, were obtained from OriGene, USA. Site-directed mutagenesis was performed using a QuikChange II kit (Agilent Technologies, USA), according to the manufacturer's protocol; KRAS mutations G12D (c.35G>A) and G13D (c.38G>G), and CTNNB1 mutation S33Y (c.98C>A) were introduced using primer sets shown in Supplementary Table 2 (“Quik_KRAS_G12D_Fw/Rev”, “Quik_KRAS_G13D_Fw/Rev” or “Quik_CTNNB_G12D_Fw/Rev”) and resulting plasmids cloned and verified by Sanger sequencing (Source Biosciences, UK). Long RNA substrates equivalent to full KRAS and CTNNB1 mRNA transcripts bearing 5′ fluorescein (“Sub_KRas_ORF” and “Sub_CTNNB1_ORF”, respectively) were prepared using HiScribe T7 and SP6 RNA synthesis kits (NEB, USA), according to the manufacturer's protocol, with a 4:1 ratio of 5′-Fluorescein-ApG dinucleotide (IBA Life Sciences, Germany) to GTP, using template plasmids linearised using XmaI (NEB, USA). Reactions were subsequently treated with TURBO DNase (Invitrogen/Thermo Fisher Scientific, USA) and RNA transcripts purified using RNeasy mini kits (Qiagen, Germany).

### 2′OMezyme Selections

Broadly, chimeric RNA-2′OMe-RNA random-sequence libraries were prepared and selected using a similar strategy as previous XNAzymes^{7, 8}. Initial library synthesis reactions were performed using 1 μM RNA primer “P1_KRas12 [G12D]”, 2 μM DNA template “N40libtemp_KRas12”, 1.3 μM 2M polymerase and 0.125 mM (each) 2′OMe-ATP, 2′OMe-CTP, 2′OMe-GTP and 2′OMe-UTP, in Thermopol buffer (NEB, USA) for 1 h at 50° C., 2 h at 65° C. MyOne Streptavidin C1 Dynabeads (Invitrogen/Thermo Fisher Scientific, USA) were used to capture (5′ biotinylated) single-stranded chimeric RNA-2′OMe-RNA libraries, allowing (unbiotinylated) DNA template to be denatured using 0.1 N NaOH and removed, as described previously⁷; libraries were subsequently purified by Urea-PAGE. Selection reactions were performed by annealing libraries in nuclease-free water (Qiagen, Germany) for 60 s at 80° C., 5 min RT then incubating at 37° C. in 2′OMezyme selection buffer (30 mM EPPS pH 7.4, 150 mM KCl, 1 mM MgCl₂). Reaction times were varied as follows: rounds 1-11; overnight (≈16 h), rounds 11 & 12; 1 h, rounds 13-15; 30 min

2′OMe-RNA reverse transcription was performed using 1 μM polymerase C8⁹, with 0.2 μM 5′ biotinylated primer “RT_Ebo” in Thermopol buffer (NEB, USA) with an additional 2 mM MgCl₂, 200 μM each dNTP, for 17 h at 65° C. First-stand cDNA was isolated using streptavidin magnetic beads (C1 MyOne, Thermo Fisher Scientific, USA), eluted by incubation in nuclease-free water for 2 min at 80° C., then amplified by a two-step nested PCR strategy using OneTaq Hot Start master mix (NEB, USA). The first ‘out nested’ PCRs used 0.5 μM forward primer “dP2_KRas12” and 0.5 μM reverse primer “RT_Ebo_out”, cycling conditions were 94° C. for 1 min, 20-35×[94° C. for 30 s, 52° C. for 30 s, 72° C. for 30 s], 72° C. for 2 min. Following the first PCR, primers were digested using ExoSAP (Ambion/Life Technologies, USA), which was then heat inactivated, according to the manufacturer's instructions. Second step (‘in-nest’) PCRs used 1 μl of unpurified out-nest PCR product as template in a 50 μl reaction with 0.5 μM forward primer “dP2_KRas12” and 0.5 μM reverse primer “RT_Ebo_in”, cycling conditions as above. Reactions were analysed by electrophoresis on 4% NGQT-1000 agarose (Thistle Scientific, UK) gels containing GelStar stain (Lonza, Switzerland). Bands of appropriate size were purified using a gel extraction kit (Qiagen, Germany) according to the manufacturer's instructions.

Purified DNA was used as the polyclonal template for either sequencing library PCR (see below) or preparative PCR (‘in-nest’ PCR scaled up to 500 μl) for generation of DNA templates for XNA synthesis. Single-stranded DNA templates were isolated using streptavidin beads and ethanol precipitated before further use.

A ‘maturation’ selection was subsequently performed for five rounds (with 30 min reactions at 37° C. in 2′OMezyme reaction buffer) using the sequence of the most abundant clone at round 15 (comprising 84,674 of 3,942,063 deep sequencing reads; ≈2%) as the basis a spiked library, synthesised as described above, using DNA template “R15_1libtemp_KRas12”. 2′OMezyme “R15/5-K” was the most abundant clone in round 5 of the maturation selection (comprising 1,291 of 5,507,023 deep sequencing reads; 0.02%).

### Deep Sequencing

Deep sequencing was performed using the MiSeq platform (Illumina, USA), as described previously⁷; 2′OMezyme selection pools were converted to sequencing libraries by PCR using primers “P5_P2_KRas12” and “P3_RT_Ebo_in” to append the necessary priming sites.

### Synthesis of 2′OMezymes for Characterisation

For initial screening of 2′OMezyme activity and evaluation of point mutations, 2′OMezymes were synthesised using polymerase 2M as described above, using RNA primer “P2_Ebo” and 3′ biotinylated DNA templates as shown in Supplementary Table 2, and isolated using MyOne Streptavidin C1 Dynabeads (Invitrogen/Thermo Fisher Scientific, USA), as described previously⁷. Following denaturation and removal of DNA template strands using 0.1 NaOH, 2′OMezymes were incubated in 0.8 N NaOH, 1 h at 65° C., to fully hydrolyse primer RNA.

2′OMezymes for all other characterisation experiments were synthesised by solid phase phosphoramidite chemistry by Merck/MilliporeSigma (Germany).

### 2′OMezyme Reactions

RNA cleavage assays were performed in trans using PAGE-purified 2′OMezymes and RNA substrates, annealed as described above and incubated at 37° C. in 2′OMezyme selection buffer (30 mM EPPS pH 7.4, 150 mM KCl, 1 mM MgCl₂), or 30 mM EPP pH 8.5, 150 mM KCl, 25 mM MgCl₂, supplemented with RNasin ribonuclease inhibitor (Promega, USA). In Mg²⁺ titration experiments, 2′OMezyme selection buffer was supplemented with additional magnesium chloride (MgCl₂); in pH titration experiments, 150 mM KCl, 1 mM MgCl₂ plus 50 mM buffer as follows was used: HEPES (pH 5.0-6.0), EPPS (pH 6.5-8.75), CHES (pH 9.0-12.0). For magnesium free reactions, 30 mM EPPS pH 7.4, 150 mM KCl, 5 mM EDTA was used.

Pseudo first-order reaction rates (kobs) under single-turnover pre-steady-state (Kₘ/k_{cat}) conditions were determined from three independent reactions with (separately annealed) catalyst at 5 μM and substrate at 1 μM, as described previously⁸, fit using Prism 9 (GraphPad Software, USA). For multiple turnover reactions, 1 μM substrate was reacted with 10 nM 2′OMezyme at 37° C. in 2′OMezyme selection buffer.

### For the Reverse RNA Ligation Reactions, the Products of a Large-Scale

“Sub_KRas_12 [G12D]” RNA cleavage reaction catalysed by 2′OMezyme “R15/5-K” were purified by Urea-PAGE and used as substrates. 5 μM 2OMezyme “R15/5-K” and 1 μM (each) of the 5′ and 3′ RNA cleavage products were annealed in water as described above, then diluted into 2′OMezyme selection buffer with or without magnesium chloride, snap-frozen on dry ice then incubated reacted at −7° C., or 37° C. for 20 h. ‘Supercooled’ samples were incubated directly at −7° C. without prior freezing on dry ice.

### Analysis of 2′OMezyme-Catalysed RNA Cleavage Products

Substrate RNA “Sub_KRas_12 [G12D]” was reacted with 2′OMezyme “R15/5-K” under selection conditions and the 5′ RNA cleavage product was purified by Urea-PAGE. The cleavage product was analysed by MALDI-ToF mass spectrometry using an Ultraflex III TOF-TOF instrument (Bruker Daltonik, Bremen, Germany) in positive ion mode as described previously⁸.

Enzymatic removal of 3′ terminal phosphates was assayed by Urea-PAGE gel shift following incubation in Calf Intestinal Phosphatase (CIP) (NEB, USA) or T4 Polynucleotide Kinase (PNK) (NEB, USA) in manufacturer's buffer for 30 min at 37° C. Hydrolysis of cyclic phosphates was achieved by incubation in 10 mM glycine pH 2.5 for 30 min at room temperature.

### Analysis of 2′OMezyme Serum Stability

PAGE-purified 2′OMezyme “R15/5-K” and DNAzyme “1023_KRasC” were annealed in water as described above, then incubated (at 5 μM) at 37° C. in 95% human serum (MilliporeSigma, Germany). Full-length catalyst remaining was quantified on Urea-PAGE gels stained with SYBR Gold (ThermoFisher Scientific, USA).

### Analysis of Aptamer Binding by Surface Plasmon Resonance (SPR)

2′OMe/MOE-RNA aptamers were synthesized from RNA primer Prim1 and 3′-biotinylated DNA template Temp_ARC224 (Supplementary Table 1) as described in section “Synthesis of 2′OMezymes for characterization” using 2′OMe/MOE-NTPs. 2′OMe/MOE-RNA aptamers were annealed at 1-10 μM in nuclease-free water by heating to 95° C. for 5 min and equilibrating at RT for 10 min. They were then diluted and analysed in PBS+0.1% (v/v) Tween20 (PBS-Tw). Surface Plasmon Resonance (SPR) measurements were made using a BIAcore 2000 instrument (GE Life Sciences, UK) at a flow rate of 20 μLmin⁻¹ at 20° C. CM4 sensor chip (GE Life Sciences, UK) surfaces were coated with Neutravidin (Pierce 31000, ThermoFisher Scientific, USA) surfaces (˜8000 RU per flow cell) using an amine coupling kit (GE Life Sciences, UK) and flowing in 5 mM NaOAc (sodium acetate), pH 5.5. Chips were equilibrated in PBS Tw and left to flow overnight until signal drift had settled. ˜2000 RU biotinylated human VEGF165 (Bio-Techne, USA) was captured (except for the reference cell) before blocking with excess free biotin. 50 μL aptamer samples at a series of concentrations (500 nM, 250 nM, 125 nM, 62.5 nM, 31.3 nM, 15.6 nM, 7.8 nM, 3.9 nM) were injected for 150 s and dissociation was recorded for 600 s, in PBS-Tw. Single injections of aptamers outside of the concentration series were performed at 100 nM (50 μL) in PBS-Tw. After every injection, the sensor surface was regenerated using two 5 μL injections of 10 mM NaOH+saline (137 mM NaCl, 2.7 mM KCl).

To obtain optimal fits, SPR data had to be fit to a double-exponential heterogeneous dissociation/association model to determine kinetic parameters from two independent datasets per aptamer with on-line reference subtraction. For the ARC224 MOE-AGC aptamer, the lowest two concentration points were not included in the analysis and discarded as outliers due to insufficient binding signal. Deviation from homogeneous 1:1 binding models is established for nucleic acid-protein interactions, and a heterogeneous model describing two conformationally divergent populations of a DNA aptamer binding VEGF has been described¹⁰.

The rate constants of dissociation and association were obtained by fitting the observed response signal R using the two equations below.

### Heterogeneous Dissociation:

$$R = {R}_{1} {e}^{- {k}_{d 1} ( t - {t}_{0} )} + ( {R}_{0} - {R}_{1} ) {e}^{- {k}_{d 2} ( t - {t}_{0} )} \qquad ( 1 )$$

where R₀ is the response at the start of dissociation (t₀), R₁ is the contribution to R₀ from component 1 (floating parameter), and therefore, (R₀−R₁) is the contribution to R₀ from component 2. K_{dh} is the dissociation rate constant for component i (floating parameter).

### Heterogeneous Association:

$$R = {R}_{eq 1} ( 1 - {e}^{- ( {k}_{a 1} C + {k}_{d 1} ) ( t - {t}_{0} )} ) + {R}_{eq 2} ( 1 - {e}^{- ( {k}_{a 2} C + {k}_{d 2} ) ( t - {t}_{0} )} ) \qquad ( 2 )$$

where R_{eqi} is the steady-state response level for component i (floating parameter), kₐᵢ is the association rate constant for component i (floating parameter), k_{di} is the dissociation rate constant for component i, C is the molar concentration of analyte, and t₀ is the start time for the association.

### NGS for 2′OMe Synthesis and RT Fidelity Analysis

For 2′OMe-RNA synthesis, ssDNA templates were generated by linearization of pASK_TGO plasmid using EcoR1 followed by by shrimp alkaline phosphatase treatment and restriction using BamHI. The 369 ntd dsDNA fragment is gel eluted and treated with lambda exonuclease (NEB) to generate single strand template for the RNA/2′OMe-RNA synthesis. The 2′OMe-RNA synthesis is carried out in 20 μL reaction volumes, modFD-N25-TGO682F primer and the ssDNA template generated as mentioned above were annealed at 95° C. for two minutes followed by 55° C. for 5 minutes in 1× Thermopol buffer containing 200 μM rNTPs or 200 μM 2′OMe-NTPs. The RNA and 2′OMe-RNA syntheses were carried out using TGK polymerase (RNA) and TGLLK or 2M or 3M (2′OMe-RNA) synthesis, respectively.

The synthesised transcripts containing 5′ biotin modification were bound to Dynabeads™ M-280 Streptavidin beads (Invitrogen) and purified by stripping off the template using 0.2 N NaOH. The magnetic beads immobilised with RNA or 2′OMe-RNA were used for reverse transcription using SSIII enzyme (ThermoFisherScientific). On bead RT reaction was performed using RT_primer TagR1-N25-TGO642R harbouring N25 internal barcode for PCR and sequencing error correction. RT reactions were carried out according to vendor's guidelines for SSIII. The cDNA bound to the RNA or 2′OMe-RNA on the beads were washed twice using 1×BWBS, stripped using 0.2 N NaOH and neutralised using Tris buffer before using for sequencing library generation. RT was repeated three more times and the eluted cDNAs were used for library preparation for deep sequencing.

The cDNAs (25 μL) were added to 50 μL PCR reaction with primers HiSeq_ModFD, forward primer and HiSeq_TagR1xx, unique barcode identifier primer (Supplementary Table 5) to demultiplex samples and to introduce adaptors for Illumina sequencing using Q5 polymerase (NEB).

Barcoded fidelity libraries were pooled and sequenced on an Illumina MiSeq for PE read of 150 cycles. Fidelity analysis was performed using the Burrows-Wheeler Aligner (BWA)11, Samtools12 and custom scripts that do the following can be found at GitHub: https://github.com/holliger-lab/fidelity-analysis. Mean error rate (Supplementary Table 4) and base substitutions were calculated for RNA and 2′OMe-RNA per 106 bases sequenced (Supplementary Tables 6 & 7).⁹

### Steady-State Kinetics

Steady-state kinetic parameters for NTP incorporation by 2M were determined by performing initial velocity measurements of single incorporations of either ATP, 2′OMe-ATP, or MOE-ATP. To generate the 2′OMe-RNA/DNA substrate, a 20-mer 2′OMe-RNA primer FD was 5′ 6-carboxyfluorescein end-labeled and annealed to the 52-mer DNA template BFL770 (Supplementary Table 1) at a 1:1.2 molar ratio. The reactions were performed at 50° C. in a mixture containing 1× Thermopol buffer, 6 mM Mg²⁺, 100 nM 2′OMe-RNA/DNA substrate, and at NTP concentrations ranging from 0.5-250 μM. Enzyme concentrations and reaction times were selected to maintain initial velocity conditions. The 25 μL reactions were stopped by addition of a quenching solution containing 100 mM EDTA, 80% deionized formamide, 0.25 mg/ml bromophenol blue and 0.25 mg/ml xylene cyanol. Moreover, less than 20% of the primers were extended as required for steady-state conditions.

Product and substrate were separated on a 22% denaturing (8 M urea) polyacrylamide gel. The resulting bands were quantified using a Cytiva Typhoon RGB imager in fluorescence mode. Steady-state kinetic parameters (K_{M}, k_{cat}) were determined by fitting the data to the Michaelis-Menten equation. The data are the means and standard error from three independent experiments.

Transcription Reactions with RGVG-M6

DNA template for transcription reactions was created by PCR-amplifying a 901-bp region on a plasmid encoding sfGFP under a T7 promoter. The PCR used 0.5 μM forward primer “5T7.for” and 0.5 μM reverse primer “pCUN_Do.rev”; cycling conditions were 95° C. for 30 s, 30×[95° C. for 10 s, 69° C. for 30 s, 72° C. for 30 s], 72° C. for 2 min.

For very permissive conditions, reactions comprised 125 nM DNA template, 200 nM T7 RNAP WT or its variant RGVG-M613, 1.5 mM MnCl2, 7.5 mM each NTP or 1 mM each 2′OMe-NTP, 0.1 U yeast inorganic pyrophosphatase. In order to compare the yield of 2′OMe-RNA synthesis by 2M and RGVG-M6, reactions were run under equimolar nucleic acid input of 0.5 pmol primer (2M) and 0.5 pmol DNA template (50 nM, RGVG-M6), and 50 nM RGVG-M6 polymerase with a polymerase:template ratio of 1:1 as described in 13. Reactions were treated with Turbo DNase and Proteinase K followed by denaturing PAGE.

### References to: Background Section, Legends of FIGS. 1 to 5, and Example 1, Results and Discussion

1. Wan W B, Seth P P. The Medicinal Chemistry of Therapeutic Oligonucleotides. J Med Chem 2016, 59(21): 9645-9667.
2. Aartsma-Rus A, Corey D R. The 10th Oligonucleotide Therapy Approved. Golodirsen for Duchenne Muscular Dystrophy Nucleic Acid Ther 2020, 30(2): 67-70.
3. Chelliserrykattil J, Ellington A D. Evolution of a T7 RNA polymerase variant that transcribes 2′-O-methyl RNA Nat Biotechnol 2004, 22(9): 1155-1160
4. Ibach J, Dietrich L, Koopmans K R, Nobel N, Skoupi M, Brakmann S. Identification of a T7 RNA polymerase variant that permits the enzymatic synthesis of fully 2′-O-methyl-modified RNA. J Biotechnol 2013, 167(3): 287-295.
5. Meyer A J, Garry D J, Hall B, Byrom M M, McDonald H G, Yang X. et al. Transcription yield of fully 2′-modified RNA can be increased by the addition of thermostabilizing mutations to T7 RNA polymerase mutants. Nucleic Acids Res 2015, 43(15): 7480-7488.
6. Burmeister P E, Lewis S D, Silva R F, Preiss J R, Horwitz L R, Pendergrast P S, et al. Direct in vitro selection of a 2′-O-methyl aptamer to VEGF. Chem Biol 2005, 12(1): 25-33.
7. Chen T, Hongdilokkul N, Liu Z., Adhikary R, Tsuen S S, Romesberg F E. Evolution of thermophilic DNA polymerases for the recognition and amplification of C2′-modified DNA. Nat Chem 2016, 8(6): 556-562.
8. Liu Z, Chen T, Romesberg F E. Evolved polymerases facilitate selection of fully 2′-OMe-modified aptamers. Chem Sci 2017, 8(12): 8179-8182.
9. Hoshino H, Kasahara Y, Kuwahara M, Obika S. DNA Polymerase Variants with High Processivity and Accuracy for Encoding and Decoding Locked Nucleic Acid Sequences. J Am Chem Soc 2020, 142(51): 21530-21537.
10. Cozens C, Mutschler H, Nelson G M, Houlihan G, Taylor A I, Holliger P. Enzymatic Synthesis of Nucleic Acids with Defined Regioisomeric 2′-5′ Linkages. Angew Chem Int Ed Engl 2015, 54(51): 15570-15573.
11. Cozens C, Pinheiro V B, Vaisman A, Woodgate R, Holliger P. A short adaptive path from DNA to RNA polymerases. Proc Natl Acad Sci USA 2012, 109(21): 8067-8072.
12. Kropp H M, Betz K, Wirth J, Diederichs K, Marx A. Crystal structures of ternary complexes of archaeal B-family DNA polymerases. PLoS One 2017, 12(12): e0188005.
13. Perera R L, Torella R, Klinge S, Kilkenny M L, 534 Maman J D, Pellegrini L. Mechanism for priming DNA synthesis by yeast DNA polymerase alpha. Elife 2013, 2: e00482.
14. Kawai G, Yamamoto Y, Kamimura T, Masegi T, Sekine M, Hata T, et al. Conformational rigidity of specific pyrimidine residues in tRNA arises from posttranscriptional modifications that enhance steric interaction between the base and the 2′-hydroxyl group. Biochemistry 1992, 31(4): 1040-1046.
15. Nishizaki T, Iwai S, Ohtsuka E, Nakamura H. Solution structure of an RNA.2′-O-methylated RNA hybrid duplex containing an RNA.DNA hybrid segment at the center. Biochemistry 1997, 36(9): 2577-2585.
16. Pinheiro V B, Taylor A I, Cozens C, Abramov M, Renders M, Zhang S, et al. Synthetic genetic polymers capable of heredity and evolution. Science 2012, 336(6079): 341-344.
17. Houlihan G, Arangundy-Franklin S, Porebski B T, Subramanian N, Taylor A I, Holliger P. Discovery and evolution of RNA and XNA reverse transcriptase function and fidelity. Nat Chem 2020, 12(8): 683-690.
18. Taylor A I, Holliger P. Directed evolution of artificial enzymes (XNAzymes) from diverse repertoires of synthetic genetic polymers. Nat Protoc 2015, 10(10): 1625-1642.
19. Egli M, Minasov G, Tereshko V, Pallan P S, Teplova M, Inamati G B, et al. Probing the influence of stereoelectronic effects on the biophysical properties of oligonucleotides: comprehensive analysis of the RNA affinity, nuclease resistance, and crystal structure of ten 2′-O-ribonucleic acid modifications. Biochemistry 2005, 44(25): 9045-9057.
20. Teplova M, Minasov G, Tereshko V, Inamati G B, Cook P D, Manoharan M, et al. Crystal structure and improved antisense properties of 2′-O-(2-methoxyethyl)-RNA. Nat Struct Biol 1999, 6(6): 535-539.
21. Khatsenko O, Morgan R, Truong L, York-Defalco C, Sasmor H, Conklin B, et al Absorption of antisense oligonucleotides in rat intestine: effect of chemistry and length. Antisense Nucleic Acid Drug Dev 2000, 10(1): 35-44.
22. Plevnik M, Cevec M, Plavec J. NMR structure of 2′-O-(2-methoxyethyl) modified and C5-methylated RNA dodecamer duplex. Biochimie 2013, 95(12): 2385-2391.
23. Martin P. Stereoselektive Synthese von 2?-O-(2-Methoxyethyl)ribonucleosiden: Nachbargruppenbeteiligung der Methoxyethoxy-Gruppe bei der Ribosylierung von Heterocyclen. Helvetica Chimica Acta 1996, 79(7): 1930-1938
24. Martin P. Ein neuer Zugang zu 2?-O-Alkylribonucleosiden und Eigenschaften deren Oligonucleotide. Helvetica Chimica Acta 1995, 78(2): 486-504.
25. Gillerman I, Fischer B. An improved one-pot synthesis of nucleoside 5′-triphosphate analogues. Nucleosides Nucleotides Nucleic Acids 2010, 29(3): 245-256.
26. Ludwig J. A new route to nucleoside 5′-triphosphates. Acta Biochim Biophys Acad Sci Hung 1981, 16(3-4): 131-133.
27. Freier S M, Altmann K H. The ups and downs of nucleic acid duplex stability: structure-stability studies on chemically-modified DNA:RNA duplexes. Nucleic Acids Res 1997, 25(22): 4429-4443.
28. Kool E T. Hydrogen bonding, base stacking, and steric effects in dna replication. Annu Rev Biophys Biomol Struct 2001, 30: 1-22.
29. Wu E Y, Beese L S. The structure of a high fidelity DNA polymerase bound to a mismatched nucleotide reveals an “ajar” intermediate conformation in the nucleotide selection mechanism. J Biol Chem 2011, 286(22): 19758-19767.
30. Wang W, Wu E Y, Hellinga H W, Beese L S. Structural factors that determine selectivity of a high fidelity DNA polymerase for deoxy-, dideoxy-, and ribonucleotides. J Biol Chem 2012, 287(34): 28215-28226.
31. Chen C Y. DNA polymerases drive DNA sequencing-by-synthesis technologies: both past and present. Front Microbiol 2014, 5: 305.
32. Redrejo-Rodriguez M, Ordonez C D, Berjon-Otero M, Moreno-Gonzalez J, Aparicio-Maldonado C, Forterre P, et al. Primer-Independent DNA Synthesis by a Family B DNA Polymerase from Self-Replicating Mobile Genetic Elements. Cell Rep 2017, 21(6): 1574-1587.
33. Blasco M A, Mendez J, Lazaro J M, Blanco L, Salas M. Primer terminus stabilization at the phi 29 DNA polymerase active site. Mutational analysis of conserved motif KXY. J Biol Chem 1995, 270(6): 2735-2740.
34. Kazlauskas D, Krupovic M, Guglielmini J, Forterre P, Venclovas C. Diversity and evolution of B-family DNA polymerases. Nucleic Acids Res 2020, 48(18): 10142-10156.
35. Franklin M C, Wang J, Steitz T A. Structure of the Replicating Complex of a Pol a Family DNA Polymerase. Cell 2001, 105(5): 657-667
36. Rudinger N Z, Kranaster R, Marx A. Hydrophobic amino acid and single-atom substitutions increase DNA polymerase selectivity. Chem Biol 2007, 14(2): 185-194.
37. Gardner A F, Jack W E. Determinants of nucleotide sugar recognition in an archaeon DNA polymerase. Nucleic Acids Res 1999, 27(12): 2545-2553.
38. Bartel D P, Szostak J W. Isolation of new ribozymes 632 from a large pool of random sequences [see comment]. Science 1993, 261(5127): 1411-1418.
39. Fedor M J. Structure and function of the hairpin ribozyme. J Mol Biol 2000, 297(2): 269-291.

### References to: Legends of FIGS. 6 to 19 (Supp. FIGS. 1 to 17), and Materials & Methods

1. Kropp H M, Betz K, Wirth J, Diederichs K, Marx A. Crystal structures of ternary complexes of archaeal B-family DNA polymerases. PLoS One 2017, 12(12): e0188005.
2. Nishizaki T, Iwai S, Ohtsuka E, Nakamura H. Solution structure of an RNA.2′-Omethylated RNA hybrid duplex containing an RNA.DNA hybrid segment at the center. Biochemistry 1997, 36(9): 2577-2585.
3. Kawai G, Yamamoto Y, Kamimura T, Masegi T, Sekine M, Hata T, et al. Conformational rigidity of specific pyrimidine residues in tRNA arises from posttranscriptional modifications that enhance steric interaction between the base and the 2′-hydroxyl group. Biochemistry 1992, 31(4): 1040-1046.
4. Skerra A. Use of the tetracycline promoter for the tightly regulated production of a murine antibody fragment in Escherichia coli. Gene 1994, 151(1-2): 131-135.
5. Cozens C, Mutschler H, Nelson G M, Houlihan G, Taylor A I, Holliger P. Enzymatic Synthesis of Nucleic Acids with Defined Regioisomeric 2′-5′ Linkages. Angew Chem Int Ed Engl 2015, 54(51): 15570-15573.
6. Pinheiro V B, Taylor A I, Cozens C, Abramov M, Renders M, Zhang S, et al. Synthetic genetic polymers capable of heredity and evolution. Science 2012, 336(6079): 341-344.
7. Taylor A I, Holliger P. Directed evolution of artificial enzymes (XNAzymes) from diverse repertoires of synthetic genetic polymers. Nat Protoc 2015, 10(10): 1625-1642.
8. Taylor A I, Pinheiro V B, Smola M J, Morgunov A S, Peak-Chew S, Cozens C, et al. Catalysts from synthetic genetic polymers. Nature 2015, 518(7539): 427-430.
9. Houlihan G, Arangundy-Franklin S, Porebski B T, Subramanian N, Taylor A I, Holliger P. Discovery and evolution of RNA and XNA reverse transcriptase function and fidelity. Nat Chem 2020, 12(8): 683-690.
10. Potty A S, Kourentzi K, Fang H, Jackson G W, Zhang X, Legge G B, et al. Biophysical characterization of DNA aptamer interactions with vascular endothelial growth factor. Biopolymers 2009, 91(2): 145-156.
11. Li H, Durbin R. Fast and accurate short read alignment with Burrows-Wheeler transform. Bioinformatics 2009, 25(14): 1754-1760.
12. Li H, Handsaker B, Wysoker A, Fennell T, Ruan J, Homer N, et al. The Sequence Alignment/Map format and SAMtools. Bioinformatics 2009, 25(16): 2078-2079.
13. Burmeister P E, Lewis S D, Silva R F, Preiss J R, Horwitz L R, Pendergrast P S, et al. Direct in vitro selection of a 2′-O-methyl aptamer to VEGF. Chem Biol 2005, 12(1): 25-33.
14. Hoshino H, Kasahara Y, Kuwahara M, Obika S. DNA Polymerase Variants with High Processivity and Accuracy for Encoding and Decoding Locked Nucleic Acid Sequences. J Am Chem Soc 2020, 142(51): 21530-21537.
15. Cozens C, Pinheiro V B, Vaisman A, Woodgate R, Holliger P. A short adaptive path from DNA to RNA polymerases. Proc Natl Acad Sci USA 2012, 109(21): 8067-8072.

Supplementary Table 1 recites, in order, SEQ ID NOs: 45 to 87.

Supplementary Table 2 recites, in order, SEQ ID NOs: 88 to 127.

Supplementary Table 5 recites, in order, SEQ ID NOs: 128 to 142.

### Supplementary Tables

### Supplementary Table 1: Primers and Templates for all Polymerase Studies, Mutagenesis, 2′OMe-RNA and MOE-RNA Synthesis, and ARC224 Aptamer Variant Synthesis.

Codons targeted for mutagenesis are highlighted in bold. Different chemistries are highlighted as follows: Black=DNA, Red=RNA, Purple=2′OMe-RNA.

| Name | Sequence 5′-3′ |
|---|---|
| TempN | CTCACGATGCTGGACCAGATAAGCACTTAGCCACGTAGTGCTGTT |
| | CGGTAATCGATCTGGCAAACGCTAATAAGGGG |
| TempNpure | CCCTCTTTCTTCCTCTTCCCGATGCTGGACCAGATAAGCACTTAGC |
| | CACGTA |
| | GTGCTGTTCGGTAATCGATCTGGCAAACGCTAATAAGGGG |
| FD (also 2′OMe- | CCCCTTATTAGCGTTTGCCA |
| RNA) | |
| FD-Test2 | CCCCUUAUUAGCGUUUGCCA GGGUAACAAGACGGAAA |
| (2′OMe-RNA) | |
| A-Test2 (also RNA, | AGGGTAACAAGACGGAAA |
| 2′OMe-RNA) | |
| Tag3.3-N40-Test2 | CCCTAGTTCTTCCTCTTCCCGCCTGTGCCACTCACTATA...N₄₀...TT |
| | TCCGTCTTGTTACCC |
| Synth-out1mm | AGGAGACAGCUAUGACAACCAAGGUAGUGCUGUUCGUGGGG |
| (2′OMe-RNA) | |
| sfGFP | CCCCTCATTAGCGTTTGCCAATGcGtAAaGGAAGCAAAGGTGAAGA |
| | ACTGTTTACCGGCGTTGTGCCGATTCTGGTGGAACTGGATGGTGA |
| | TGTGAATGGCCATAAATTTAGCGTTCGTGGCGAAGGCGAAGGTGA |
| | TGCGACCAACGGTAAACTGACCCTGAAATTTATTTGCACCACCGG |
| | TAAACTGCCGGTTCCGTGGCCGACCCTGGTGACCACCCTGACCT |
| | ATGGCGTTCAGTGCTTTAGCCGCTATCCGGATCATATGAAACGCC |
| | ATGATTTCTTTAAAAGCGCGATGCCGGAAGGCTATGTGCAGGAAC |
| | GTACCATTAGCTTCAAAGATGATGGCACCTATAAAACCCGTGCGG |
| | AAGTTAAATTTGAAGGCGATACCCTGGTGAACCGCATTGAACTGA |
| | AAGGTATTGATTTTAAAGAAGATGGCAACATTCTGGGTCATAAACT |
| | GGAATATAATTTCAACAGCCATAACGTGTATATTACCGCCGATAAA |
| | CAGAAAAATGGCATCAAAGCGAACTTTAAAATCCGTCACAACGTG |
| | GAAGATGGTAGCGTGCAGCTGGCGGATCATTATCAGCAGAATACC |
| | CCGATTGGTGATGGCCCGGTGCTGCTGCCGGATAATCATTATCTG |
| | AGCACCCAGAGCGTTCTGAGCAAAGATCCGAATGAAAAACGTGAT |
| | CATATGGTGCTGCTGGAATTTGTTACCGCCGCGGGCATTACCCAC |
| | GGTATGGATGAACTGTATAAAGGCAGCTAACCCCACGAACAGCAC |
| | TACCTTG |
| DIGN25 | DIG-GATAAGCACTTAGCC |
| D540X_for | GACTCA GGTCTC TAC GCG NNS ACA GAT GGA TTT TTG GCA |
| D540X_rev | GACTCA GGTCTC CGC GTA GAG GAC TTT AAA |
| T541X_for | GACTCA GGTCTC GCG GAC NNS GAT GGA TTT TTG GCA ACA |
| T541X_rev | GACTCA GGTCTC GTC CGC GTA GAG GAC TTT |
| K592X_fo | GACTCA GGTCTC ACG AAG NNS AAG TAC GCG GTT ATA GAC |
| K592X_ba | GACTCA GGTCTC CTT CGT CAC GAA GAA GCC |
| D614X_fo | GACTCA GGTCTC AGG CGT NNS TGG AGC GAG ATA GCG AAG |
| D614X_ba | GACTCA GGTCTC ACG CCT AAC TAT TTC AAG |
| E664X_fo | GACTCA GGTCTC ATC TAC NNS CAG ATA ACC CGC GAC |
| E664X_ba | GACTCA GGTCTC GTA GAT GAC CAG CTT CTC |
| HisTag_fo | GACTCA GGTCTC AAG ACA GGC AGC GGC AGC GGC AGC CAT |
| | CAC CAT CAC CAT CAC TAA TAA GTC GAC CTG CAG |
| HisTag_ba | GACTCA GGTCTC TGT CTT AGG TTT TAG CCA C |
| L521H_for | GACTCA GGTCTC CAG TAC CAC GAG ACT ACG ATA AGG GAA |
| | ATA |
| L521H_rev | GACTCA GGTCTC GTA CTG CCT GCC CCA |
| T541G_for | GACTCA GGTCTC GCG GAC GGG GAT GGA TTT TTG GCA ACA |
| | ATA |
| K592A_for | GACTCA GGTCTC ACG AAG GCC AAG TAC GCG GTT ATA GAC |
| | GAG |
| K664R_for | GACTCA GGTCTC ATC TAC CGG CAG ATA ACC CGC GAC |
| Prim 1 (RNA) | AAUCUACCACAUCGCUCAUUG |
| Temp_ARC224 | CGAATGCGCGACTTCTCAAACTGCATATCG |
| | CAATGAGCGATGTGGTAGATT-TEG-biotin |
| A141D_A143E_KOD_ | GAGCTGAAAATGCTAGCATTCGATATTGAAACTCTCTACCATGAGG |
| fwd | GCGAGGAG |
| A141D_A143E_KOD_ | CTCCTCGCCCTCATGGTAGAGAGTTTCAATATCGAATGCTAGCATT |
| rev | TTCAGCTC |
| D614N_KOD_fwd | CTTGAGATTGTGAGGCGTAACTGGAGCGAGATAGCGAAAG |
| D614N_KOD_rev | CTTTCGCTATCTCGCTCCAGTTACGCCTCACAATCTCAAG |
| L521I_KOD_fwd | GTAACGGCCTGGGGAAGGGAGTACATTACGATGACCATCAAG |
| L521I_KOD_rev | CTTGATGGTCATCGTAATGTACTCCCTTCCCCAGGCCGTTAC |
| L545F_KOD_fwd | CAGCGACACCGACGGATTTTTTGCCACAATACCTGGAG |
| L545F_KOD_rev | CTCCAGGTATTGTGGCAAAAAATCCGTCGGTGTCGCTG |
| N210D_KOD_fwd | GACGTTCTCATAACCTACGATGGTGACAACTTCGACTTC |
| N210D_KOD_rev | GAAGTCGAAGTTGTCACCATCGTAGGTTATGAGAACGTC |
| Q93V_KOD_fwd | CTACTTTACTCATCCACAGGACGTGCCAGCAATAAGGGACAAGAT |
| | AC |
| Q93V_KOD_rev | GTATCTTGTCCCTTATTGCTGGCACGTCCTGTGGATGAGTAAAGTA |
| | G |
| BFL770 | TCGATACTGGTACTAATGATTAACGATGTTGGCAAACGCTAATAAG |
| | GGGTCG |
| 5T7.for | GATCGATCTCGCCCGCGAAATTAATACGACTCACTATAGG |
| pCUN_Do.rev | GCGGCTCTTCCTCTCTCATCCGCCGGCCGACTAGT |

### Supplementary Table 2: Primer and Template Sequences for 2′OMezymes.

Different chemistries are highlighted as follows: Black=DNA, Red=RNA, Purple=2′OMe-RNA (NB—2′OMe-RNA oligos shown here were prepared by solid phase synthesis, not by polymerase).

| Name | Sequence (5′-3′) | Notes |
|---|---|---|
| P1_KRas_12[G12D] | [BiotinTEG][internal Cy3][C18 spacer] | Primer/substrate for |
| | [C18 spacer]UAGUUGGAGCUGAUGGCGUAGGCA | 2′OMezyme library synthesis; |
| | AGAGUG | KRAS c.23-52. |
| | | ″12″ refers to the target codon. |
| N40libtemp_KRas12 | TGGCGTAGGCAAGAGTGCTACGCC...N₄₀... | Template for 2′OMezyme |
| | CTCCAACTCAACGACCAGTACACAAG | degenerate library synthesis. |
| RT_Ebo | [BiotinTEG]CTCTGCTACTCGTGGTCCTTGTG | Primer for 2′OMezyme library |
| | TACTGGTCGTTG | reverse transcription (RT). |
| dP2_KRas | [BiotinTEG]TGGCGTAGGCAAGAGTG | Forward primer for library / |
| | (& version without 5′ Biotin) | template preparation PCRs. |
| RT_Ebo_out | CTCTGCTACTCGTGGTC | Reverse primer for semi- |
| | | nested (1^{st}) library RT-PCR. |
| RT_Ebo_in | CTTGTGTACTGGTCGTTG | Reverse primer for (2^{nd}) library |
| | | RT-PCR. |
| R15_1libtemp_ | CTTGTGTACTGGTCGTTGGGTTGGAGCAGT | Template for 2′OMezyme |
| KRas12 | CTGTCATGCCTTCTCTGTCCATCATGGTTCA | patterned library synthesis. |
| | CGTGCGTCACTCTTGCCTACGCCA | |
| | (underlined region = 70% base shown + | |
| | 10% each of the other three) | |
| R15/5-K | CCUACGCACGUGACCCAUGACGGACAGAG | RNA endonuclease |
| | AAGGCAUGACAGACUGCUCCAA | 2′OMezyme specific for KRAS |
| | | RNA c.26 - 44. |
| 1023_KRasC | CGCCATGGCTAGCTACAACGAAGCTCCA | RNA endonuclease DNAzyme |
| | | ″10-23″ re-targeted to KRAS |
| | | RNa. |
| Sub_KRas_12[wt] | [6FAM]UAGUUGGAGCUGGUGGCGUAGGCA | Substrate RNA equivalent to |
| | AGAGUG | KRAS c.23-52. |
| Sub_KRas_12 | [6FAM]UAGUUGGAGCUGAUGGCGUAGGCA | Substrate RNA equivalent to |
| [G12D] | AGAGUG | KRAS c.23-52, with G12D |
| | | [c.35 G > A] mutation. |
| P2_Ebo | [6FAM]AAUCUACCACAUCGCUCAUUG | Primer for 2′OMezyme |
| | | synthesis. |
| R15/5- | TACCTGGAAGTCTGTCATGCCTTCTCTGTC | Template for synthesis of |
| C_U45temp_Ebo | CGTCATGGGTCACGTGAATCCATCAATGAG | ″R15/5-C″ 2′OMezyme (with |
| | CGATGTGGTAGATT[BiotinTEG] | U45); R15/5-C retargeted to |
| | | CTNNB1 RNA c.88-108.. |
| R15/5- | TACCTGGAAGTCTGTCATGCCTTCTCTGTC | Template for synthesis of |
| C_A9Ctemp_Ebo | CGTCATGGGTCACGGGAATCCATCAATGAG | ″R15/5-C″ 2′OMezyme with |
| | CGATGTGGTAGATT[BiotinTEG] | mutation A9C. |
| R15/5- | TACCTGGAAGTCTGTCATGCCTTCTCTGTC | Template for synthesis of |
| C_A9Gtemp_Ebo | CGTCATGGGTCACGCGAATCCATCAATGAG | ″R15/5-C″ 2′OMezyme with |
| | CGATGTGGTAGATT[BiotinTEG] | mutation A9G. |
| R15/5- | TACCTGGAAGTCTGTCATGCCTTCTCTGTC | Template for synthesis of |
| C_A9Utemp_Ebo | CGTCATGGGTCACGAGAATCCATCAATGAG | ″R15/5-C″ 2′OMezyme with |
| | CGATGTGGTAGATT[BiotinTEG] | mutation A9U. |
| R15/5- | TACCTGGAAGTCTGGCATGCCTTCTCTGTC | Template for synthesis of |
| C_A39Ctemp_Ebo | CGTCATGGGTCACGTGAATCCATCAATGAG | ″R15/5-C″ 2′OMezyme with |
| | CGATGTGGTAGATT[BiotinTEG] | mutation A39C. |
| R15/5- | TACCTGGAAGTCTGCCATGCCTTCTCTGTC | Template for synthesis of |
| C_A39Gtemp_Ebo | CGTCATGGGTCACGTGAATCCATCAATGAG | ″R15/5-C″ 2′OMezyme with |
| | CGATGTGGTAGATT[BiotinTEG] | mutation A39G. |
| R15/5- | TACCTGGAAGTCTGACATGCCTTCTCTGTC | Template for synthesis of |
| C_A39Utemp_Ebo | CGTCATGGGTCACGTGAATCCATCAATGAG | ″R15/5-C″ 2′OMezyme with |
| | CGATGTGGTAGATT[BiotinTEG] | mutation A39U. |
| R15/5- | TACCTGGAAGTTTGTCATGCCTTCTCTGTCC | Template for synthesis of |
| C_G42Atemp_Ebo | GTCATGGGTCACGTGAATCCATCAATGAGC | ″R15/5-C″ 2′OMezyme with |
| | GATGTGGTAGATT[BiotinTEG] | mutation A39C. |
| R15/5- | TACCTGGAAGTGTGTCATGCCTTCTCTGTC | Template for synthesis of |
| C_G42Ctemp_Ebo | CGTCATGGGTCACGTGAATCCATCAATGAG | ″R15/5-C″ 2′OMezyme with |
| | CGATGTGGTAGATT[BiotinTEG] | mutation G42C. |
| R15/5- | TACCTGGAAGTATGTCATGCCTTCTCTGTC | Template for synthesis of |
| C_G42Utemp_Ebo | CGTCATGGGTCACGTGAATCCATCAATGAG | ″R15/5-C″ 2′OMezyme with |
| | CGATGTGGTAGATT[BiotinTEG] | mutation G42U. |
| R15/5- | TACCTGGATGTCTGTCATGCCTTCTCTGTC | Template for synthesis of |
| C_U45Atemp_Ebo | CGTCATGGGTCACGTGAATCCATCAATGAG | ″R15/5-C″ 2′OMezyme with |
| | CGATGTGGTAGATT[BiotinTEG] | mutation U45A. |
| R15/5- | TACCTGGAGGTCTGTCATGCCTTCTCTGTC | Template for synthesis of |
| C_U45Ctemp_Ebo | CGTCATGGGTCACGTGAATCCATCAATGAG | ″R15/5-C″ 2′OMezyme with |
| | CGATGTGGTAGATT[BiotinTEG] | mutation U45C. |
| R15/5- | TACCTGGACGTCTGTCATGCCTTCTCTGTC | Template for synthesis of |
| C_U45Gtemp_Ebo | CGTCATGGGTCACGTGAATCCATCAATGAG | ″R15/-C″ 2′OMezyme with |
| | CGATGTGGTAGATT[BiotinTEG] | mutation U45G. |
| R15/5- | TACCTGGATGTCTGCCATGCCTTCTCTGTC | Template for synthesis of |
| C_A39G_U45Atemp_ | CGTCATGGGTCACGTGAATCCATCAATGAG | ″R15/5-C″ 2′OMezyme with |
| Ebo | CGATGTGGTAGATT[BiotinTEG] | mutation A39G. |
| R15/5-C [U45A] | ATGGATTCACGUGACCCAUGACGGACAGA | RNA endonuclease |
| | GAAGGCAUGACAGACATCCAGGTA | 2′OMezyme specific for |
| | | CTNNB1 RNA c.88-108. |
| Sub_CTNNB1_33 | [6FAM]UCUUACCUGGACUCUGGAAUCCAU | Substrate RNA equivalent to |
| [wt] | UCU | CTNNB1 c.85-111. |
| Sub_CTNNB1_33 | [6FAM]UCUUACCUGGACUAUGGAAUCCAUU | Substrate RNA equivalent to |
| [S33Y] | CU | CTNNB1 c.85-111, with |
| | | S33Y [c.98 C > A] mutation. |
| Quik_KRas_G12D_ | TGGTAGTTGGAGCTGATGGCGTAGGCAAG | Forward primer for KRAS |
| Fw | AG | G12D [c.35G > A] |
| | | mutagenesis. |
| Quik_KRas_G12D | CTCTTGCCTACGCCATCAGCTCCAACTACC | Reverse primer for KRAS |
| Rev | A | G12D [c.35G > A] |
| | | mutagenesis. |
| Quik_KRas_G13D_ | TAGTTGGAGCTGGTGACGTAGGCAAGAGT | Forward primer for KRAS |
| Fw | GC | G13D [c.38G >A] |
| | | mutagenesis. |
| Quik_KRas_G13D_ | GCACTCTTGCCTACGTCACCAGCTCCAACT | Reverse primer for KRAS |
| Rev | A | G13D [c.38G > A] |
| | | mutagenesis. |
| Quik_CTNNB_S33Y_ | AGCAACAGTCTTACCTGGACTATGGAATCC | Forward primer for CTNNB1 |
| Fw | ATTCTG | S33Y [c.98C > A] mutagenesis. |
| Quik_CTNNB_S33Y_ | CAGAATGGATTCCATAGTCCAGGTAAGACT | Forward primer for CTNNB1 |
| Rev | GTTGCT | S33Y [c.98C > A] mutagenesis. |
| P5_P2_KRas12 | AATGATACGGCGACCACCGAGATCTACACT | Forward primer for preparing |
| | CTTTCCCTACACGACGCTCTTCCGATCTNN | Miseq libraries |
| | NATTCCTTGGCGTAGGCAAGAGTG | (six-letter barcode highlighted; |
| | | can be changed for others). |
| P3_RT_Ebo_in | CAAGCAGAAGACGGCATACGAGATCGGTCT | Reverse primer for preparing |
| | CGGCATTCCTGCTGAACCGCTCTTCCGATC | Miseq libraries. |
| | TCTTGTGTACTGGTCGTTG | |
| R15/5-C_4M_temp | TACCTGGATGTCTGCCATGCTTTCTCTGTC | Template for synthesis of |
| | CGTCATGTGTCACGTGAATCCATCAATGAG | ″R15/5-C″ 2′OMezyme with |
| | CGATGTGGTAGATT[BiotinTEG] | mutations A39G, U45A, |
| | | G33A, C16A. |
| R15/5-C_5M_temp | TACCTGGATTTCTGCCATGCTTTCTCTGTCC | Template for synthesis of |
| | GTCATGTGTCACGTGAATCCATCAATGAGC | ″R15/5-C″ 2′OMezyme with |
| | GATGTGGTAGATT[BiotinTEG] | mutations A39G, U45A, |
| | | G33A, C16A, C44A. |
| R15/5-C_6M_temp | TACCTGGATTTCTTCCATGCTTTCTCTGTCC | Template for synthesis of |
| | GTCATGTGTCACGTGAATCCATCAATGAGC | ″R15/5-C″ 2′OMezyme with |
| | GATGTGGTAGATT[BiotinTEG] | mutations A39G, U45A, |
| | | G33A, C16A, C44A, C40A. |

### Supplementary Table 3: Kinetic Data Obtained Through Fit of SPR Curves.

Every row of fitted parameters is obtained from the fit of one concentration series (eight individual injections in two-fold dilution series; MOE-AGC: six individual injections; as described in Materials & Methods). Shown is the standard error of the mean (s.e.m.).

| | | | | | | | | overall |
|---|---|---|---|---|---|---|---|---|
| | | | | | | | mean | mean |
| Aptamer | kₐ₁ [M⁻¹ s⁻¹] | k_{d1} [s⁻¹] | K_{D1} [M] | kₐ₂ [M⁻¹ s⁻¹] | k_{d2} [s⁻¹] | K_{D2} [M] | K_{D} [nM] | K_{D} [nM] |
| ARC224 | 1.7 · 10⁵± | 4.8 · 10⁻³± | 2.8 · 10⁻⁸± | 2.3 · 10⁵ ± | 5.8 · 10⁻³± | 2.5 · 10⁻⁸ ± | 27 ± 15 | 23 ± 16 |
| 2′OMe | 2.2 · 10⁵ | 4.0 · 10⁻³ | 1.8 · 10⁻⁸ | 4.4 · 10⁵ | 4.8 · 10⁻³ | 1.1 · 10⁻⁸ | | |
| | 1.9 · 10⁵± | 6.9 · 10⁻³± | 3.7 · 10⁻⁸± | 2.3 · 10⁶ ± | 4.3 · 10⁻³± | 1.9 · 10⁻⁹ ± | 19 ± 18 | |
| | 1.4 · 10⁵ | 5.1 · 10⁻³ | 3.6 · 10⁻⁸ | 5.8 · 10⁶ | 3.2 · 10⁻³ | 5.5 · 10⁻¹⁰ | | |
| ARC224 | 1.1 · 10⁵± | 4.4 · 10⁻³± | 4.0 · 10⁻⁸± | 1.3 · 10⁵ ± | 6.5 · 10⁻³± | 4.8 · 10⁻⁸ ± | 44 ± 45 | 30± 29 |
| MOE- | 8.8 · 10⁴ | 3.7 · 10⁻³ | 4.2 · 10⁻⁸ | 1.8 · 10⁵ | 8.7 · 10⁻³ | 4.8 · 10⁻⁸ | | |
| AC | 2.7 · 10⁵± | 4.7 · 10⁻³± | 1.7 · 10⁻⁸± | 1.6 · 10⁵ ± | 2.3 · 10⁻³± | 1.4 · 10⁻⁸ ± | 16 ± 13 | |
| | 4.9 · 10⁵ | 3.7 · 10⁻³ | 7.4 · 10⁻⁹ | 1.6 · 10⁵ | 2.9 · 10⁻³ | 1.8 · 10⁻⁸ | | |
| ARC224 | 1.4 · 10⁵± | 1.1 · 10⁻²± | 7.8 · 10⁻⁸± | 7.4 · 10⁴ ± | 8.9 · 10⁻³± | 1.2 · 10⁻⁷ ± | 99 ± 99 | 92 ± 88 |
| MOE- | 6.3 · 10⁴ | 7.7 · 10⁻³ | 1.2 · 10⁻⁷ | 8.0 · 10⁴ | 6.0 · 10⁻³ | 7.5 · 10⁻⁸ | | |
| AGC | 1.1 · 10⁵± | 7.9 · 10⁻³± | 7.5 · 10⁻⁸± | 1.6 · 10⁵ ± | 1.5 · 10⁻²± | 9.3 · 10⁻⁸ ± | 84 ± 77 | |
| | 5.8 · 10⁴ | 4.5 · 10⁻³ | 7.8 · 10⁻⁸ | 1.3 · 10⁵ | 9.8 · 10⁻³ | 7.7 · 10⁻⁸ | | |

SUPPLEMENTARY TABLE 4
Fidelity of 2′OMe-RNA synthesis (RNA synthesis for TGK)
as measured by barcoded Next Generation Sequencing (NGS)⁹

| | Experiment 1: | Experiment 2: | Experiment 3: | Experiment 4: | | |
|---|---|---|---|---|---|---|
| | error rate | error rate | error rate | error rate | Total | Mean |
| | (bases | (bases | (bases | (bases | bases | error rate |
| Polymerase | sequenced) | sequenced) | sequenced) | sequenced) | sequenced | (×10⁻³) |
| TGK (RNA) | 2.09 × 10⁻³ | 2.53 × 10⁻³ | 1.06 × 10⁻³ | 1.84 × 10⁻³ | 8.9 × 10⁵ | 1.9 ±/− 0.6^{a} |
| | (2.9 × 10⁵) | (4.4 × 10⁴) | (1.1 × 10⁵) | (4.7 × 10⁵) | | |
| TGLLK | 1.61 × 10⁻³ | 6.87 × 10⁻⁴ | 1.93 × 10⁻³ | 1.65 × 10⁻³ | 4.7 × 10⁶ | 1.5 +/− 0.5 |
| | (1 6 × 10⁶) | (2.8 × 10⁶) | (1.35 × 10⁵) | (8.7 × 10⁴) | | |
| 2M | 8.17 × 10⁻⁴ | 4.99 × 10⁻⁴ | 1.00 × 10⁻³ | 2.63 × 10⁻⁴ | 1.3 × 10⁶ | 0.65 +/− 0.3 |
| | (1.2 × 10⁶) | (2.0 × 10⁴) | (5.1 × 10⁴) | (7.2 × 10⁴) | | |
| 3M | 1.35 × 10⁻³ | 1.77 × 10⁻³ | 9.67 × 10⁻⁴ | 1.20 × 10⁻³ | 4.1 × 10⁵ | 1.3 +/− 0.6 |
| | (2.2 × 10⁵) | (4.1 × 10⁴) | (2.3 × 10⁴) | (1.4 × 10⁵) | | |

^{a} TGK has a published fidelity of 1.03 × 10⁻³ (mean error rate)¹⁵ for RNA synthesis

Supplementary Table 5:
Primer and template sequences for RNA and 2′OMe-RNA Fidelity.
Note: The reverse primer for sequencing libraries carries six-letter barcode upstream to NNN
for demultiplexing the samples for analysis.

| Name | Sequence (5′-3′) | Notes |
|---|---|---|
| modFD-N25-TGO682F | /52Bio//iSp18//iFluorT/CCTATCGCGG | Primer for the RNA or |
| | TATCGGAATCCCNNNNNNNNNNNN | 2′Ome RNA synthesis |
| | NNNNNNNNNNNNNCTTCCGAGGAT | |
| | GAACTTGA | |
| TagR1-N25-TGO642R | /5TYE665/ACATCTCGTGCGCATCTG | RT primer with N25 |
| | CANNNNNNNNNNNNNNNNNNNNNN | barcode/Unique modifier |
| | NNNTACAACGGCGACAACTTC | index sequence for error |
| | | correction |
| Hiseq_R1c_7 | AATGATACGGCGACCACCGAGATC | Reverse primer for |
| | TACACTCTTTCCCTACACGACGCTC | preparing Sequencing |
| | TTCCGATCT NNN CAGATC | libraries. |
| | ACATCTCGTGCGCATCTGCA | |
| Hiseq_R1c_8 | AATGATACGGCGACCACCGAGATC | Reverse primer for |
| | TACACTCTTTCCCTACACGACGCTC | preparing Sequencing |
| | TTCCGATCT NNN ACTTGA | libraries. |
| | ACATCTCGTGCGCATCTGCA | |
| Hiseq_R1c_9 | AATGATACGGCGACCACCGAGATC | Reverse primer for |
| | TACACTCTTTCCCTACACGACGCTC | preparing Sequencing |
| | TTCCGATCT NNN GATCAG | libraries. |
| | ACATCTCGTGCGCATCTGCA | |
| Hiseq_R1c_10 | AATGATACGGCGACCACCGAGATC | Reverse primer for |
| | TACACTCTTTCCCTACACGACGCTC | preparing Sequencing |
| | TTCCGATCT NNN TAGCTT | libraries. |
| | ACATCTCGTGCGCATCTGCA | |
| Hiseq_R1c_11 | AATGATACGGCGACCACCGAGATC | Reverse primer for |
| | TACACTCTTTCCCTACACGACGCTC | preparing Sequencing |
| | TTCCGATCT NNN GGCTAC | libraries. |
| | ACATCTCGTGCGCATCTGCA | |
| Hiseq_R1c_12 | AATGATACGGCGACCACCGAGATC | Reverse primer for |
| | TACACTCTTTCCCTACACGACGCTC | preparing Sequencing |
| | TTCCGATCT NNN CTTGTA | libraries. |
| | ACATCTCGTGCGCATCTGCA | |
| Hiseq_R1c_13 | AATGATACGGCGACCACCGAGATC | Reverse primer for |
| | TACACTCTTTCCCTACACGACGCTC | preparing Sequencing |
| | TTCCGATCT NNN AGTCAA | libraries. |
| | ACATCTCGTGCGCATCTGCA | |
| Hiseq_R1c_14 | AATGATACGGCGACCACCGAGATC | Reverse primer for |
| | TACACTCTTTCCCTACACGACGCTC | preparing Sequencing |
| | TTCCGATCT NNN AGTTCC | libraries. |
| | ACATCTCGTGCGCATCTGCA | |
| Hiseq_R1_15 | AATGATACGGCGACCACCGAGATC | Reverse primer for |
| | TACACTCTTTCCCTACACGACGCTC | preparing Sequencing |
| | TTCCGATCT NNN ATGTCA | libraries. |
| | ACATCTCGTGCGCATCTGCA | |
| Hiseq_R1_16 | AATGATACGGCGACCACCGAGATC | Reverse primer for |
| | TACACTCTTTCCCTACACGACGCTC | preparing Sequencing |
| | TTCCGATCT NNN CCGTCC | libraries. |
| | ACATCTCGTGCGCATCTGCA | |
| Hiseq_R1_17 | AATGATACGGCGACCACCGAGATC | Reverse primer for |
| | TACACTCTTTCCCTACACGACGCTC | preparing Sequencing |
| | TTCCGATCT NNN GTAGAG | libraries. |
| | ACATCTCGTGCGCATCTGCA | |
| Hiseq_R1_18 | AATGATACGGCGACCACCGAGATC | Reverse primer for |
| | TACACTCTTTCCCTACACGACGCTC | preparing Sequencing |
| | TTCCGATCT NNN GTCCGC | libraries. |
| | ACATCTCGTGCGCATCTGCA | |
| HiSeq_ModFD | CAAGCAGAAGACGGCATACGAGAT | Forward primer for |
| | CGTGATGTGACTGGAGTTCAGACG | preparing sequencing |
| | TGTGCTCTTCCGATCCCTATCGCG | libraries |
| | GTATCGGAATC | |

SUPPLEMENTARY TABLE 6
Total number of substitutions during 2′ OMe synthesis and RT by SSIII per 10⁶ bases

| Polymerase | 2′OMeC −> T | 2′OMeG −> A | 2′OMeU −> C | 2′OMeA −> G | 2′OMeG −> T | 2′OMeC −> A |
|---|---|---|---|---|---|---|
| TGLLK/SIII | 64 | 184 | 76 | 10 | 216 | 24 |
| 3M/SIII | 188 | 179 | 222 | 120 | 212 | 88 |
| 2M/SIII | 23 | 44 | 44 | 5 | 16 | 5 |
| Chem | 1521 | 1219 | 608 | 656 | 89 | 3211 |
| 2′ OMe^{a} | | | | | | |
| Polymerase | 2′OMeG −> C | 2′OMeU −> A | 2′OMeA −> T | 2′OMeC −> G | 2′OMeA −> C | 2′OMeU −> G |
| TGLLK/SIII | 224 | 40 | 9 | 1 | 609 | 1 |
| 3M/SIII | 58 | 6 | 55 | 34 | 67 | 1 |
| 2M/SIII | 16 | 7 | 5 | 0 | 425 | 0 |
| Chem | 90 | 625 | 190 | 196 | 778 | 46 |
| 2′ OMe^{a} | | | | | | |

^{a}Chemical/solid-phase synthesised 2′OMe-RNA published data for reference

SUPPLEMENTARY TABLE 7
Total number of substitutions during RNA
synthesis and RT per 106 bases sequenced.

| Polymerase | rC −> T | rG −> A | rU −> C | rA −> G | rG −> T | rC −> A |
|---|---|---|---|---|---|---|
| TGK / SIII | 341 | 605 | 53 | 108 | 274 | 152 |
| mRNA/RT521K* | 442 | 104 | 46 | 600 | 34 | 1130 |
| mRNA / SSIII* | 19 | 10 | 5 | 15 | 1 | 5 |

| Polymerase | rG −> C | rU −> A | rA −> T | rC −> G | rA −> C | rU −> G |
|---|---|---|---|---|---|---|
| TGK / SIII | 121 | 5 | 62 | 26 | 50 | 4 |
| mRNA/RT521K* | 2 | 540 | 162 | 280 | 96 | 216 |
| mRNA / SSIII* | 1 | 2 | 3 | 4 | 1 | 0 |

*cellular mRNA transcripts used for RT published data for reference

1-29. (canceled)

30. A nucleic acid polymerase capable of producing a non-DNA nucleotide polymer from a nucleic acid template,
said polymerase comprising amino acid sequence having at least 36% identity to the amino acid sequence of SEQ ID NO: 1,
wherein said amino acid sequence is mutated relative to the amino acid sequence of SEQ ID NO: 1 at T541 and/or K592, and wherein the amino acid sequence is mutated relative to the amino acid sequence of SEQ ID NO: 1 at E664.

31. The nucleic acid polymerase of claim 30, wherein the amino acid sequence comprises: i) a T541 mutation and a K592 mutation, ii) a T541 mutation and a E664 mutation, or iii) a T541 mutation, a K592 mutation, and a E664 mutation.

32. The nucleic acid polymerase of claim 30, wherein the T541 mutation is T541G, T541S, T541A, T541C, T541D, T541P, or T541N.

33. The nucleic acid polymerase of claim 30, wherein the K592 mutation is K592G, K592A, K592C, K592M, K592S, K592D, K592P, K592N, K592T, K592E, K592V, K592Q, K592H, K592I, or K592L.

34. The nucleic acid polymerase of claim 30, wherein the E664 mutation is E664H, E664K, or E664R.

35. The nucleic acid polymerase of claim 30, wherein the amino acid sequence comprises the mutations T541G and K592A.

36. The nucleic acid polymerase of claim 30, wherein the amino acid sequence comprises:
i) one or more, or all, of the following mutations: V93Q, D141A, E143A, and A485L relative to SEQ ID NO: 1; and/or
ii) one or more, or all, of the following mutations: Y409, I521, and F545 relative to SEQ ID NO: 1; and/or
iii) one or more, or all, of the following mutations: Y409G, I521L or I521H, and F545L relative to SEQ ID NO: 1.

37. The nucleic acid polymerase of claim 30, wherein the amino acid sequence comprises a D614 mutation relative to SEQ ID NO: 1.

38. The nucleic acid polymerase of claim 30, wherein said amino acid sequence has at least 50%, 60%, 70%, 80%, 90%, 95%, 99%, or 100% similarity or identity to the amino acid sequence of SEQ ID NO: 1.

39. The nucleic acid polymerase of claim 30, wherein said amino acid sequence has at least 36%, 50%, 60%, 70%, 80%, 90%, 95%, 99%, or 100% similarity or identity to:
i) the amino acid sequence of SEQ ID NO: 3 or SEQ ID NO: 4, wherein residues 93, 141, 143, 409, 485, 521, 541, 545, 592, and 664 are invariant; and/or
ii) the amino acid sequence of SEQ ID NO: 5 or SEQ ID NO: 6, wherein residues 93, 141, 143, 409, 485, 521, 541, 545, 592, 614, and 664 are invariant.

40. The nucleic acid polymerase of claim 30, wherein said amino acid sequence comprises SEQ ID NO: 7 or SEQ ID NO: 8.

41. A nucleic acid polymerase capable of producing a non-DNA nucleotide polymer from a nucleic acid template,
said polymerase comprising amino acid sequence having at least 36% identity to the amino acid sequence of SEQ ID NO: 1,
wherein said amino acid sequence is mutated relative to the amino acid sequence of SEQ ID NO: 1 at E664R.

42. The nucleic acid polymerase of claim 30, wherein the amino acid sequence comprises one or more, or any combination, of the following mutations: D540, D542, K591, K593, Y663, and Q665 relative to SEQ ID NO: 1.

43. A nucleic acid polymerase capable of producing a non-DNA nucleotide polymer from a nucleic acid template,
said polymerase comprising amino acid sequence having at least 36% identity to the amino acid sequence of SEQ ID NO: 1,
wherein said amino acid sequence is mutated relative to the amino acid sequence of SEQ ID NO: 1 at one of, all of, or any combination of positions D540, D542, K591, K593, Y663, and/or Q665 relative to SEQ ID NO: 1.

44. The nucleic acid polymerase of claim 42, wherein:
(i) the mutation at D540 is D540A, D540G, D540S, or D540C; and/or
(ii) the mutation at D542 is D542A, D542G, D542S, or D542C; and/or
(iii) the mutation at K591 is K591G, K591A, K591C, K591M, K591S, K591D, K591P, K591N, K591T, K591E, K591V, K591Q, K591H, K591I, or K591L; and/or
(iv) the mutation at K593 is K593G, K593A, K593C, K593M, K593S, K593D, K593P, K593N, K593T, K593E, K593V, K593Q, K593H, K593I, or K593L; and/or
(v) the mutation at Y663 is Y663K, Y663R, or Y663H; and/or
(vi) the mutation at Q665 is Q665K, Q665R, or Q665H.

45. The nucleic acid polymerase of claim 30, wherein the non-DNA nucleotide polymer comprises 2′-O-methyl-RNA and (2′OMe-RNA) nucleotides and/or 2′-O-(2-methoxyethyl)-RNA (MOE-RNA) nucleotides.

46. The nucleic acid polymerase of claim 30, wherein the amino acid sequence is derived from the wild type sequence of a nucleic acid polymerase of the polB family.

47. A method for making a non-DNA nucleotide polymer, said method comprising:
contacting a nucleic acid template with a nucleic acid polymerase of claim 30, under conditions conducive to polymerisation.

48. A nucleic acid encoding a polymerase according to claim 30.

49. A host cell comprising a polymerase according to claim 30.

50. A host cell comprising a nucleic acid according to claim 48.
